(19) United States
(12) Patent Application Publication
(10) Pub. No.: US 2008/0213175 A1
(43) Pub. Date: Sep. 4, 2008

(54) Click chemistry-derived cyclic peptidomimetics as integrin markers

(76) Inventors: Hartmuth C. Kolb, Playa Del Rey, CA (US); Kai Chen, Los Angeles, CA (US); Joseph C. Walsh, Pacific Palisades, CA (US); Umesh Gangadharmath, Los Angeles, CA (US); Dhanalakshmi Kasi, Los Angeles, CA (US); Bing Wang, San Jose, CA (US); Brian Duclos, Los Angeles, CA (US); Qianwa Liang, Hacienda Heights, CA (US); Henry Clifton Padgett, Hermosa Beach, CA (US); Farhad Karimi, Canton, MA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/901,704

(22) Filed: Sep. 17, 2007

Related U.S. Application Data
(60) Provisional application No. 60/844,807, filed on Sep. 15, 2006.

(51) Int. Cl. A61K 51/08; C07K 5/12; A61K 38/12; A61K 38/14

(52) U.S. Cl. 424/1.69; 530/317; 514/11; 514/10; 514/8

(57) ABSTRACT

The present application is directed to radiolabeled cyclic peptidomimetics, pharmaceutical compositions comprising radiolabeled cyclic peptidomimetics, and methods of using the radiolabeled cyclic peptidomimetics. Such peptidomimetics can be used in imaging studies, such as Positron Emitting Tomography (PET) or Single Photon Emission Computed Tomography (SPECT).

### RELATED APPLICATION

This application claims priority to U.S. Provisional Application No. 60/844,807, filed Sep. 15, 2006, the content of which is hereby incorporated in its entirety by reference.

### FIELD OF THE INVENTION

The present application deals with radiolabeled cyclic peptidomimetics, pharmaceutical compositions comprising radiolabeled cyclic peptidomimetics, and methods of using the radiolabeled cyclic peptidomimetics. The present application is further directed to methods of preparing the radiolabeled cyclic peptidomimetics. Such peptidomimetics can be used in imaging studies, such as Positron Emitting Tomography (PET) or Single Photon Emission Computed Tomography (SPECT).

In particular this application discloses the preparation and use of radiolabeled cyclic peptidomimetics for imaging integrins (e.g., integrin αvβ3) in vivo. Click chemistry is utilized to attach a radiolabel to cyclopeptidomimetics that contain an Arg-Gly-Asp (RGD) fragment and that further carry various hydrophilic linkages, such as oligo- or poly-ethyleneglycol (“PEG”) moieties, polar amino acid moieties, sugars, or sugar mimetics, such as cyclohexane diols or polyols. One advantage disclosed in the present application is a click chemistry labeling step that is easy to perform, that is fast and provides high yields of radiolabeled products that are easy to purify. The binding affinities of the radiolabeled peptidomimetics for different integrins have been determined using biochemical in vitro assays, such as cell-binding assays or surface plasmon resonance assays. The click chemistry-derived cyclic peptidomimetics of the present application display surprisingly high binding affinities to the biological target, and demonstrate very favorable pharmacokinetic behavior in mice (e.g. high tumor uptake and fast clearance through predominantly renal-routes).

### BACKGROUND OF THE INVENTION

Non-invasive molecular imaging plays a key role in detection of disease by characterizing and measuring biological processes at the molecular level. A number of medical diagnostic procedures, including Positron Emission Tomography (PET), and Single Photon Emission Computed Tomography (SPECT) utilize radiolabeled compounds. PET and SPECT are very sensitive techniques and require small quantities of radiolabeled compounds, called tracers. The tracers are comprised of a positron-emitting isotope, such as F-18, C-11, N-13, or O-15, and a ligand, which binds specifically and with high affinity to the target, such as tumor-specific molecular marker. The labeled compounds are transported, accumulated and converted in vivo in exactly the same way as the corresponding non-radioactively compound. Tracers, or probes, can be radiolabeled with a radionuclide useful for PET imaging, such as ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I and ¹³¹I, or with a radionuclide useful for SPECT imaging, such as ⁹⁹Tc, ⁷⁵Br, ⁶¹Cu, ¹⁵³Gd, ¹²⁵I, ¹³¹I and ³²P.

PET is a molecular imaging technology which creates images based on the distribution of molecular imaging tracers carrying positron-emitting isotopes in the tissue of the patient. The PET method has the potential to detect malfunction on a cellular level in the investigated tissues or organs. PET has been used in clinical oncology, such as for the imaging of tumors and metastases, and has been used for diagnosis of certain brain diseases, as well as mapping brain and heart function. Similarly, SPECT can be used to complement any gamma imaging study, where a true 3D representation can be helpful, for example, imaging tumor, infection (leukocyte), thyroid, or bones.

Angiogenesis, the formation of new blood vessels by sprouting from existing blood vessels, is a fundamental process that occurs during tumor progression. Angiogenesis is regulated by a balance between pro-angiogenic factors, such as vascular endothelial growth factor (VEGF), epidermal growth factor (EGF), fibroblast growth factor (FGF), and anti-angiogenic molecules, such as angiostatin and endostatin. Most tumors begin growing as avascular dormant nodules until they reach steady-state populations of proliferating and apoptosing cells. Angiogenesis starts with perivascular detachment and vessel dilation, followed by angiogenic sprouting, new vessel formation, maturation, and the recruitment of perivascular cells. Blood vessel formation continues as the tumor grows, feeding on hypoxic and necrotic areas of the tumor for essential nutrients and oxygen. This multistep process offers several targets for therapeutic interventions. Thus, great efforts are being made to develop anti-angiogenic drugs for cancer treatment and prevention of tumor recurrence and metastasis.

Integrins, which are largely responsible for cell-cell and cell-matrix interactions, are one of the main classes of receptors regulating tumor metastasis and angiogenesis. In addition to having adhesive functions, integrins transduce messages via various signaling pathways and influence proliferation and apoptosis of tumor cells, as well as of activated endothielial cells. Research has shown that integrins are a family of adhesion molecules consisting of two noncovalently bound transmembrane subunits (α and β). Both are type I membrane proteins with large extracellular segments that pair to create heterodimers with distinct adhesive capabilities. In mammals, 18α and 8β subunits assemble into 24 different receptors. One prominent member of this receptor class is the integrin αᵥβ₃. The special role of integrin αᵥβ₃ in tumor invasion and metastasis arises from its ability to recruit and activate matrix metalloproteinases 2 (MMP-2) and plasmin, which degrade components of the basement membrane and interstitial matrix. It has been demonstrated that tumor expression of integrin αᵥβ₃ correlates well with tumor progression in several malignancies such as melanoma, glioma, breast cancer, and ovarian cancer. Since it is not readily detectable in quiescent vessels but becomes highly expressed in angiogenic vessels, integrin αᵥβ₃ serves as an excellent molecular marker for tumor metastasis and angiogenesis imaging. Thus, the ability to noninvasively visualize and quantify integrin αᵥβ₃ expression level will provide new opportunities to document tumor integrin expression, to properly select patients for anti-integrin treatment, and to monitor treatment efficacy in integrin-positive patients.

Besides αᵥβ₃, αᵥβ₅ integrin has been implicated in the angiogenic process possibly via a signaling pathway distinct from that of αᵥβ₃. Indeed, neutralizing anti-αᵥβ₅ antibody inhibits VEGF-stimulated angiogenesis in the chorioallantoic membrane assay, whereas anti-αᵥβ₃ antibody inhibits FGF2-induced angiogenesis. The existence of distinct angiogenic pathways can be explained by the prevalence of specific growth factors and/or cell-adhesive proteins in different conditions. Thus, experimental evidence suggests that dual αᵥβ₃/αᵥβ₅ antagonists may represent a multi-target approach for the inhibition of tumor angiogenesis and tumor growth.

Based on the findings that several extracellular matrix proteins, such as vitronectin, fibrinogen, and thrombospondin interact via the RGD sequence with the integrins, linear as well as cyclic peptides containing the RGD sequence have been introduced. Kessler and co-workers [1] developed the pentapeptide cyclo(-Arg-Gly-Asp-D-Phe-Val-) (“c(RGDfV)”) which showed high affinity and selectivity for integrin αᵥβ₃. To date, most integrin targeted PET studies have been focused on radiolabeling of RGD peptide antagonists of integrin αvβ3 due to its relatively high binding affinity.

Several groups are focused on the modification of the linkage connecting cyclic RGD peptide to the radionuclide [2-4]. Currently, [¹⁸F]Galacto-RGD represents a promising integrin marker in the clinical trial arena.

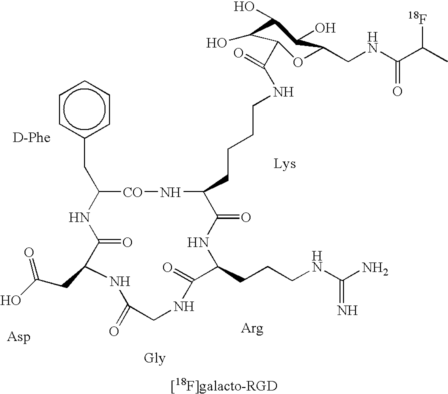

It was demonstrated that [¹⁸F]galacto-RGD exhibited integrin αᵥβ₃-specific tumor uptake in integrin-positive M21 melanoma xenograft model [5-7 see also 18]. Moreover, [¹⁸F]galacto-RGD was sensitive enough for visualization of integrin αᵥβ₃ expression resulting exclusively from the tumor vasculature using an A431 human squamous cell carcinoma model, in which the tumor cells are integrin negative. Initial clinical trials in healthy volunteers and a limited number of cancer patients revealed that this tracer could be safely administered to patients and was able to delineate certain lesions that were integrin-positive with reasonable contrast.

As a monomeric RGD peptide tracer, [¹⁸F]galacto-RGD has relatively low tumor targeting efficacy; clinical use of this tracer is severely limited because of its relatively low integrin binding affinity, modest tumor standard uptake values, and unfavorable pharmacokinetic behavior. Therefore, tumors with low integrin expression level may not be detectable. In addition, prominent activity accumulation in the liver, kidneys, spleen, and intestines was observed in both preclinical models and human studies. As a result, it was difficult to visualize lesions in the abdomen. This tracer is also very difficult to synthesize, thereby limiting its availability. Strategies for improving pharmacokinetic behavior as well as tumor uptake and retention pattern of peptides with an RGD motif include introduction of hydrophilic amino acids, conjugation of PEG (poly(ethyleneglycol)) and multimerisation of RGD.

In order to obtain favorable pharmacokinetics and tumor uptake, the use of conformationally constrained cyclic peptides or relatively rigid peptidomimetic scaffolds that match biologically active conformations has been shown to enhance ligand binding for entropic reasons in various systems. Cyclic RGD peptide lends itself well to such structural modification, e.g. incorporating peptide mimics into the cyclic RGD-containing backbone. Recently, a library of RGD-containing pseudopeptides has been synthesized [8]. These compounds are characterized by the replacement in cyclo[Arg-Gly-Asp-D-Phe-Val] of the D-Phe-Val or the D-Phe-[NMe]Val dipeptide with a 6,5- and 7,5-fused bicyclic lactam, such as for example, compounds of the formula:

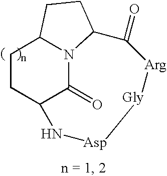

In comparison with D-Phe-Val or D-Phe-[NMe]Val dipeptide, the bicyclic lactams show different reverse-turn mimetic properties that constrain the RGD sequence into different conformations and provide the required activity and selectivity for integrin antagonism. These cyclic peptidomimetics cannot be employed easily as PET imaging tracers due to their strenuous synthetic procedure.

### SUMMARY OF THE INVENTION

Applicants observed that despite a few good examples of RGD-containing tracers, several key challenges remain to be resolved. Firstly, the pharmacokinetic behavior of the tracer needs to be improved. Secondly, a major drawback of the strategies examined by others is that the radiolabeling process is very difficult to perform, which limits the exploration of improved derivatives and the use of these imaging agents as standard clinical biomarkers.

Applicants have found that substitution of an amide bond in a cyclic polypeptide, e.g. c(RGDfK), by a 5 or 6 membered heterocycle, such as a 1,4-disubstituted 1,2,3-triazole (“1,2,3-anti-triazole”) preserves the cyclic peptides' functional and structural integrity while providing enhance metabolic stability in vivo. In this fashion, problems with pharmacokinetic behavior can be attenuated. A library of cyclic peptidomimetics was prepared using a technique known as click chemistry [9-17]. Click chemistry is a high-yielding and modular approach and as such, the pharmacokinetic properties of the cyclopeptide analogs of the present application are easily modified. In particular, the click chemistry-functionalized cyclic peptidomimetics of the present application may be readily prepared by solid or solution phase peptide synthesis techniques, as disclosed herein.

The present application discloses effective imaging agents developed for detecting blood vessel growth in tumors (angiogenesis) in vivo. In the labeled cyclic peptidomimetics of the present application, RGD-containing mimetics carry polar residues on a pendantside chain; generally those polar residues are coupled with a moiety comprising a radionuclide via a ‘click chemistry’ linkage (i.e. a 1,4- or 1,5-disubstituted 1,2,3-triazole). The labeled cyclic peptidomimetics of the present application are easy to both synthesize and radiolabel using click chemistry. The compounds demonstrate surprisingly high binding affinity to integrin αᵥβ₃, and good pharmacokinetic properties. The imaging agents disclosed in the present application are used as a marker for imaging integrins in vivo. More specifically, this application discloses a means for detecting blood vessel growth in certain cancers in vivo, as well as a means for monitoring the efficacy of cancer therapy. Since the imaging agent allows in vivo imaging of blood vessel growth in solid tumors, it enables personalized anti-angiogenesis cancer therapies.

To solve the problem of low signal to noise ratios, a library of cyclic peptidomimetics, assembled using click chemistry, was built using the RGD sequence as an integrin binding motif. The binding affinities of the cyclic peptidomimetics for different integrins have been determined using biochemical in vitro assays, such as cell-binding assays or surface plasmon resonance assays. The cyclic peptidomimetics that display high binding affinity are selected as candidates for radiolabeling, or conjugation with appropriate linker moieties and radionuclide such as [18F]-fluorine for in vivo PET imaging.

### DETAILED DESCRIPTION

The embodiments of the invention and the various features and advantageous details thereof are explained more fully with reference to the non-limiting embodiments and examples that are described and/or illustrated in the accompanying drawings and detailed in the following description. It should be noted that the features of one embodiment may be employed with other embodiments as the skilled artisan would recognize, even if not explicitly stated herein. The examples used herein are intended merely to facilitate an understanding of ways in which the invention may be practiced and to further enable those of skill in the art to practice the embodiments of the present application. Accordingly, the examples and embodiments herein should not be construed as limiting the scope of the application, which is defined solely by the appended claims.

### BRIEF DESCRIPTION OF THE FIGURES

FIG. 1 Binding affinity comparison of Compound 1 and RGDFK using surface plasmon resonance assay.

FIG. 2 Binding affinity comparison of Compounds 10, 13 and GalactosylRGDfK using cell-based integrin αvβ3 binding competition assay.

FIG. 3A is a time course imaging using micro-PET imaging of Compound 2 in a U87MG Xenograft Mouse Model.

FIG. 3B is a graph of ratio of tumor to major organ uptake over time of Compound 2 in a U87MG Xenograft Mouse Model.

FIG. 4A is a time course imaging of Compound 2 in A431 Xenograft Mouse Model.

FIG.4B is a graph of ratio of tumor to major organ uptake over time of Compound 2 in A431 Xenograft Mouse Model.

FIG. 5A is a time course imaging of Compound 3 in U87MG Xenograft Mouse Model.

FIG. 5B is a graph of ratio of tumor to major organ uptake over time of Compound 3 in U87MG Xenograft Mouse Model.

FIG. 6A is a time course imaging of Compound 3 in A427 Xenograft Mouse Model.

FIG. 6B is a graph of ratio of tumor to major organ uptake over time of Compound 3 in A427 Xenograft Mouse Model.

FIG. 7 is a graph of distribution data of Compound 2 in U87MG tumor-bearing mice.

FIG. 8A are graphs from a metabolic stability studies of Compound 2 in mice by radio-HPLC.

FIG. 8B is a graph from biodistribution studies of Compound 2 in mice.

FIG. 9A are graphs from a metabolic stability studies of Compound 3 in mice by radio-HPLC.

FIG. 9B is a graph from biodistribution studies of Compound 3 in mice.

### DEFINITIONS

Unless specifically noted otherwise herein, the definitions of the terms used are standard definitions used in the art of organic and peptide synthesis and pharmaceutical sciences.

An “alkyl” group is a straight, branched, saturated or unsaturated, aliphatic group having a chain of carbon atoms, optionally with oxygen, nitrogen or sulfur atoms inserted between the carbon atoms in the chain or as indicated. Alkyl groups may be optionally substituted. A (C₁-C₆)alkyl, for example, includes alkyl groups that have a chain of between 1 and 6 carbon atoms, and include, for example, the groups methyl, ethyl, propyl, isopropyl, vinyl, allyl, 1-propenyl, isopropenyl, ethynyl, 1-propynyl, 2-propynyl, 1,3-butadienyl, penta-1,3-dienyl, and the like. An alkyl group, such as a “C₁-C₆ alkyl,” that forms a part of a group or a linker that is a divalent alkyl group, i.e. that is attached to two other moiety, may also be referred to as an “alkylene” or a “alkylenyl” group. Similarly, an alkenyl group, alkynyl group, aryl group, etc in a structure that is shown as a divalent group may be referred to as an alkenylenyl, alkynylenyl, arylenyl group respectively.

An alkyl as noted with another group such as an aryl group, represented as “arylalkyl” for example, is intended to be a straight, branched, saturated or unsaturated aliphatic divalent group with the number of atoms indicated in the alkyl group (as in (C₁-C₆)alkyl, for example) and/or aryl group or when no atoms are indicated means a bond between the aryl and the alkyl group. Nonexclusive examples of such group include benzyl, phenylethyl and the like.

An “alkylene” group or “alkylenyl group” is a straight, branched, saturated or unsaturated aliphatic divalent group with the number of atoms indicated in the alkyl group; for example, a —(C₁-C₃)alkylene- or —(C₁-C₃)alkylenyl-.

The term “alkenyl” refers to unsaturated groups which contain at least one carbon-carbon double bond and includes straight-chain, branched-chain and cyclic groups. Alkene groups may be optionally substituted. Exemplary groups include 1-butenyl, 2-butenyl, 3-butenyl, isobutenyl, 1-propenyl, 2-propenyl, and ethenyl.

The term “alkynyl” refers to unsaturated groups which contain at least one carbon-carbon triple bond and includes straight-chain, branched-chain and cyclic groups. Alkyne groups may be optionally substituted. Exemplary groups include 1-butynyl, 2-butynyl, 3-butynyl, 1-propynyl, 2-propynyl and ethynyl.

The term “carbocycle” (or carbocyclyl) as used herein refers to a C₃ to C₈ monocyclic, saturated, partially saturated or aromatic ring. Bonds in a carbocycle depicted with a “ - - - ” indicate bonds that can be either single or double bonds. Carbocycles may be optionally substituted. Non-exclusive examples of carbocycle include cyclopropane, cyclobutane, cyclopentane, cyclohexane, cycloheptane, cyclopentene, cyclohexene, cycloheptene, cyclooctene, benzyl, naphthene, anthracene, phenanthracene, biphenyl and pyrene.

A “heterocycle” is a carbocycle group wherein one or more of the atoms forming the ring is a heteroatom that is a N, O, or S. Bonds in a heterocycle depicted with a “ - - - ” indicate bonds that can be either single or double bonds consistent with the valency requirements based on the atoms comprising the heterocycle. The heterocycle may be saturated, partially saturated or aromatic. Heterocycles may be optionally substituted. Non-exclusive examples of heterocyclyl (or heterocycle) include piperidyl, 4-morpholyl, 4-piperazinyl, pyrrolidinyl, 1,4-diazaperhydroepinyl, acetonidyl-4-one, 1,3-dioxanyl, thiophenyl, furanyl, pyrrolyl, pyrazolyl, pyridinyl, pyrimidinyl, pyridazinyl, pyranyl and the like.

The term “alkoxy” or “alkyloxy” includes linear or branched alkyl groups that are attached to divalent oxygen. The alkyl group is as defined above. Examples of such substituents include methoxy, ethoxy, t-butoxy, and the like. The term “alkoxyalkyl” refers to an alkyl group that is substituted with one or more alkoxy groups. Alkoxy groups may be optionally substituted. The term “aryloxy” refers to an aryl group that is attached to an oxygen, such as phenyl-O—, etc.

The term “optionally substituted” or “substituted” refers to the specific group wherein one to four hydrogen atoms in the group may be replaced by one to four substituents, independently selected from alkyl, aryl, alkylene-aryl, hydroxy, alkoxy, aryloxy, perhaloalkoxy, heterocycle, azido, amino, guanidino, amidino, halo, alkylthio, oxo, acylalkyl, carboxy esters, carboxyl, carboxamido, nitro, acyloxy, aminoalkyl, alkylaminoaryl, alkylaminoalkyl, alkoxyaryl, arylamino, phosphono, sulfonyl, carboxamidoaryl, hydroxyalkyl, haloalkyl, cyano, alkoxyalkyl, and perhaloalkyl. In addition, the term “optionally substituted” or “substituted” in reference to R₂ or R₃ includes groups substituted by one to four substitutents, as identified above, that further comprise a positron or gamma emitter. Such positron emitters include, but are not limited to, ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, ¹³¹I, ⁹⁹Tc, ⁷⁵Br, ¹⁵³Gd and ³²P.

As used herein, the term “peptidomimetic” refers to a molecule that mimics the structural and/or functional features of a peptide. In particular, in the peptidomimetics of the present application, an amide bond in a cyclic polypeptide, e.g. c(RGDfK), is replaced with one or more 5 or 6 membered heterocycles, such as a 1,2,3-triazole. The peptidomimetics of the present application preserve the cyclic peptides' functional and structural integrity and generally enhance the cyclic peptides' metabolic stability in vivo.

As used herein, the term “side chain” of a natural or unnatural amino acid refers to “Q” group in the amino acid formula, as exemplify with NH₂CH(Q)CO₂H.

As used herein, the term “polar amino acid moiety” refers to the side chain, Q, of a polar natural or unnatural amino acid. Polar natural amino acids include but are not limited to arginine, asparagine, aspartic acid, cysteine, glutamic acid, glutamine, histidine and lysine.

As used herein, “natural amino acid” refers to the naturally occurring amino acids: glycine, alanine, valine, leucine, isoleucine, serine, methionine, threonine, phenylalanine, tyrosine, tryptophan, cysteine, proline, histidine, aspartic acid, asparagine, glutamic acid, glutamine, arginine and lysine.

The term “unnatural amino acid” refers to any derivative of a natural amino acid including for example D and L forms, and α- and β-amino acid derivatives. It is noted that certain amino acids, e.g., hydroxyproline, that are classified as a non-natural amino acid herein, may be found in nature within a certain organism or a particular protein. The following non-exclusive examples of non-natural amino acids and amino acid derivatives may be used according to the application (common abbreviations in parentheses): β-alanine (β-ALA), γ-aminobutyric acid (GABA), ornithine, 2-aminobutyric acid (2-Abu), α,β-dehydro-2-aminobutyric acid (8-AU), 1-aminocyclopropane-1-carboxylic acid (ACPC), aminoisobutyric acid (Aib), γ-carboxyglutamic acid, 2-amino-thiazoline-4-carboxylic acid, 5-aminovaleric acid (5-Ava), 6-aminohexanoic acid (6-Ahx), 8-aminooctanoic acid (8-Aoc), 11-aminoundecanoic acid (11-Aun), 12-aminododecanoic acid (12-Ado), 2-aminobenzoic acid (2-Abz), 3-aminobenzoic acid (3-Abz), 4-aminobenzoic acid (4-Abz), 4-amino-3-hydroxy-6-methylheptanoic acid (Statine, Sta), aminooxyacetic acid (Aoa), 2-aminotetraline-2-carboxylic acid (ATC), 4-amino-5-cyclohexyl-3-hydroxypentanoic acid (ACHPA), para-aminophenylalanine (4-NH₂-Phe), biphenylalanine (Bip), para-bromophenylalanine (4-Br-Phe), ortho-chlorophenylalanine] (2-Cl-Phe), meta-chlorophenylalanine (3-Cl-Phe), para-chlorophenylalanine (4-Cl-Phe), meta-chlorotyrosine (3-Cl-Tyr), para-benzoylphenylalanine (Bpa), tert-butylglycine (TLG), cyclohexylalanine (Cha), cyclohexylglycine (Chg), 2,3-diaminopropionic acid (Dpr), 2,4-diaminobutyric acid (Dbu), 3,4-dichlorophenylalanine (3,4-Cl₂-Phe), 3,4-diflurorphenylalanine (3,4-F₂-Phe), 3,5-diiodotyrosine (3,5-I₂-Tyr), ortho-fluorophenylalanine (2-F-Phe), meta-fluorophenylalanine (3-F-Phe), para-fluorophenylalanine (4-F-Phe), meta-fluorotyrosine (3-F-Tyr), homoserine (Hse), homophenylalanine (Hfe), homotyrosine (Htyr), 5-hydroxytryptophan (5-OH-Trp), hydroxyproline (Hyp), para-iodophenylalanine (4-I-Phe), 3-iodotyrosine (3-I-Tyr), indoline-2-carboxylic acid (Idc), isonipecotic acid (Inp), meta-methyltyrosine (3-Me-Tyr), 1-naphthylalanine (1-Nal), 2-naphthylalanine (2-Nal), para-nitrophenylalanine (4-NO₂-Phe), 3-nitrotyrosine (3-NO₂-Tyr), norleucine (Nle), norvaline (Nva), ornithine (Orn), ortho-phosphotyrosine (H₂PO₃-Tyr), octahydroindole-2-carboxylic acid (Oic), penicillamine (Pen), pentafluorophenylalanine (F₅-Phe), phenylglycine (Phg), pipecolic acid (Pip), propargylglycine (Pra), pyroglutamic acid (PGLU), sarcosine (Sar), tetrahydroisoquinoline-3-carboxylic acid (Tic), thienylalanine, and thiazolidine-4-carboxylic acid (thioproline, Th). Additionally, N-alkylated amino acids may be used, as well as amino acids having amine-containing side chains (such as Lys and Orn) in which the amine has been acylated or alkylated.

As used herein, “sugar moiety” refers to an oxidized, reduced or substituted saccharide monoradical or diradical covalently attached via any atom(s) of the sugar moiety. Representative sugars include, by way of illustration, hexoses such as D-glucose, D-mannose, D-xylose, D-galactose, vancosamine, 3-desmethyl-vancosamine, 3-epi-vancosamine, 4-epi-vancosamine, acosamine, actinosamine, daunosamine, 3-epi-daunosamine, ristosamine, D-glucamine, N-methyl-D-glucamine, D-glucuronic acid, N-acetyl-D-glucosamine, N-acetyl-D-galactosamine, sialyic acid, iduronic acid, L-fucose, and the like; pentoses such as D-ribose or D-arabinose; ketoses such as D-ribulose or D-fructose; disaccharides such as 2-O-((α-L-vancosaminyl)-β-D-glucopyranose, 2-O-(3-desmethyl-α-L-vancosaminyl)-β-D-glucopyranose, sucrose, lactose, or maltose; derivatives such as acetals, amines, acylated, sulfated and phosphorylated sugars; and oligosaccharides having from 2 to 10 sugar units.

As used herein, a hexose structure that is represented below, for example:

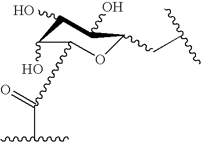

showing the curved lines [symbol]
is intended to represent a structure having the stereochemistry of any one of the natural sugars, including allose, altrose, galactose, glucose, gulose, idose, mannose, talose, etc . . . , as well as their unnatural and synthetic hexose analogs and derivatives, and also includes certain sugar moieties.

As used herein, “sugar mimetic” refers to a carbocycle or a heterocycle substituted with at least one hydroxyl group. Such carbocycle groups include, but are not limited to cyclohexane, cyclohexene, cyclopentane and cyclobutane; such heterocycles include, but are not limited to, pyrrolidine and piperidine.

As used herein, “PEG moiety” refers to a fragment of poly (ethylene glycol), a polymer of ethylene oxide. PEG has the formula:

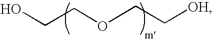

where m′ is an integer between 1 and 200, alternatively between 1 and 110 or between 10 and 90; m′ can also be an integer between 50 and 75. Alternately m′ can be an integer between 1 and 50 or even between 1 and 15.

“Linker” as used herein refers to a chain comprising 1 to 200 atoms and may comprise atoms or groups, such as C, —NR—, O, S, —S(O)—, —S(O)₂—, CO, —C(NR)—, a PEG moeity, and the like, wherein R is H or is selected from the group consisting of (C₁₋₁₀)alkyl, (C₃₋₈)cycloalkyl, aryl(C₁₋₅)alkyl, heteroaryl(C₁₋₅)alkyl, amino, aryl, heteroaryl, hydroxy, (C₁₋₁₀)alkoxy, aryloxy, heteroaryloxy, each substituted or unsubstituted. The linker chain may also comprise part of a saturated, unsaturated or aromatic ring, including monocyclic (e.g. a 1,5-cyclohexylenyl group, sugar mimetic, sugar moiety etc . . . ), polycyclic and heteroaromatic rings (e.g. a 2,4-pyridinyl group etc. . . . ). The representation of “(C₁₋₃)alkyl”, for example, is used interchangeably with “C₁-C₃alkyl” to mean the same. As used herein, the term “linker” may be used to link interconnecting moieties such as —X—W—VR₂R₃, —Y—W—VR₂R₃, -Z—W—VR₂R₃, etc. . . . , including linking a cyclic polypeptide moiety and a triazole moiety.

As used herein, where a divalent group, such as a linker, is represented by a structure -A-B—, as shown below, it is intended to also represent a group that may be attached in both possible permutations, as noted in the two structures below.

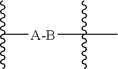

may also be

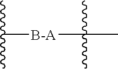

As used herein, the phrase “pharmaceutically acceptable carrier” refers to an excipient that may optionally be included in the compositions of the present application and that causes no significant adverse toxicological effects when administered in vivo.

As used herein, the term “patient” refers to any warm-blooded animal, such as a mouse, dog or human.

The compounds of the present application may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4- hydroxybenzoic, phenylacetic, mandelic, embonic (pamoic), methanesulfonic, ethanesulfonic, benzenesulfon/c, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N, NT-dibenzylethylenediamine, chloroprocaine, choline, diethanolamine, ethylenediamine, meglumine-(N-methylglucamine) and procaine.

### Embodiments, Aspects and Variations of the Invention

The present application provides the following embodiments, aspects and variations:

One aspect of the present application is a peptidomimetic of formula I:

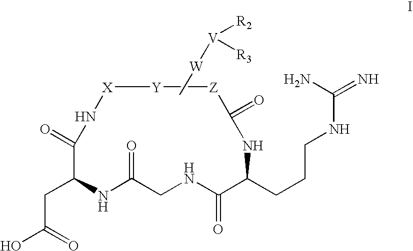

wherein W is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;

V is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;

wherein at least one, but not both of W and V is a 5- or 6-membered heterocycle;

X is selected from the group consisting of —C₁-C₆ alkyl-(5- to 6-membered heterocycle)-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

Y is selected from the group consisting of 5- or 6-membered heterocycle, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

Z is selected from the group consisting of -(5- or 6-membered heterocycle)-C₁-C₆ alkyl-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

any one of X, Y, or Z but not more than one of X, Y and Z is a 5- or 6-membered heterocycle;

where each R₁ is independently selected from the group consisting of a side chain of a natural amino acid and a side chain of an unnatural amino acid, wherein the natural amino acid and the unnatural amino acid is either in the D or L form;

R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene, carbocycle and heterocycle groups are each optionally substituted; and optionally the fragment W—V(R₂)(R₃) is absent; wherein at least one of W, X, Y, Z, R₂, and R₃ comprises a radionuclide selected from the group consisting of positron or gamma emitters.

In certain variations of each of the embodiments and aspects of the present application, the 5-membered heterocycle is a substituted 1,2,3-triazolyl group as disclosed herein.

In one embodiment of any of the aspects disclosed herein, V is a 5-membered heterocycle; and W is a linker either comprising a sugar mimetic selected from the group consisting of a 4 to 6-membered carbocycle substituted with at least one hydroxyl group and a 5- to 6-membered heterocycle substituted with at least one hydroxyl group or comprising a sugar moiety selected from the group consisting of glucose and galactose. In another embodiment, V is 1,2,3-triazolyl, W is a linker comprising a sugar mimetic selected from the group consisting of a hydroxylated cyclohexanyl group, a hydroxylated cyclopentanyl group, a hydroxylated pyrrolidinyl group, and a hydroxylated piperidinyl group.

In one embodiment of any aspect of the present application, Y is a 5-membered heterocycle; V is a 5-membered heterocycle; each of X and Z is a linker selected from the group consisting of comprising —C(H)(R₁)—, and optionally substituted C₁-C₆ alkyl; the radionuclide is selected from the group consisting of ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, ¹³¹I, ⁹⁹Tc, ⁷⁵Br, ¹⁵³Gd and ³²P.

In one embodiment of any of the aspects of the present application, Y is a 5- or 6-membered heterocycle; V is a 5-membered heterocycle; each of X and Z is a linker selected from the group consisting of comprising C₁-C₆ alkyl, C₂-C₆ alkenyl, C₂-C₆ alkynyl, aryl, aryl-(C₁-C₆ alkylene)- wherein the alkyl, alkenyl, alkynyl, aryl-alkylene groups are each optionally substituted; and the radionuclide is selected from the group consisting of ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, ¹³¹I, ⁹⁹Tc, ⁷⁵Br, ¹⁵³Gd and ³²P. In another embodiment, W is selected from the group consisting of:

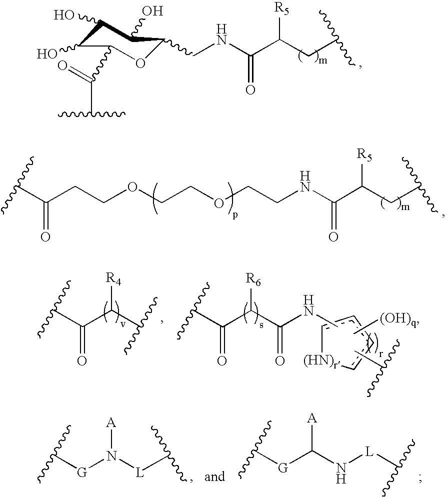

where R₄ is selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, C₁-C₆-alkoxy-C₁-C₆-alkyl, and a PEG moiety, wherein the alkyl, alkenyl, alkynyl, alkyloxy, aryl, carbocycle, and heterocycle groups are each optionally substituted;

R₅ is selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, and C₁-C₆-alkoxy-C₁-C₆-alkyl, wherein the alkyl, alkenyl, alkynyl, alkyloxy, aryl, carbocycle, and heterocycle groups are each optionally substituted;

each R₆ is independently selected from the group consisting of —H, —OH, C₁-C₆ alkyl, C₂-C₆ alkenyl, C₂-C₆ alkynyl, C₁-C₆ alkyloxy, aryl-(C₁-C₆ alkylene)-, hydroxy-C₁-C-₆-alkyl, and C₁-C₆-alkoxy-C₁-C₆-alkyl, wherein the alkyl, alkenyl, alkynyl, alkyloxy, and aryl-alkylene groups are each optionally substituted;

G is selected from the group consisting of:

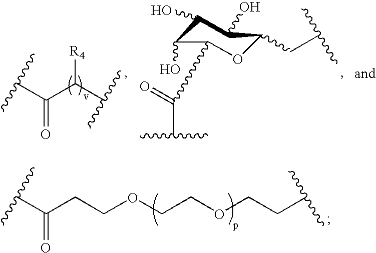

L is selected from the group consisting of:

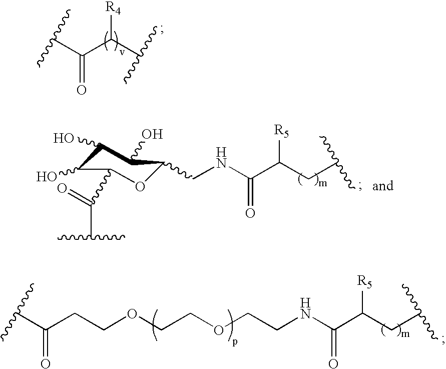

A is selected from the group consisting of:

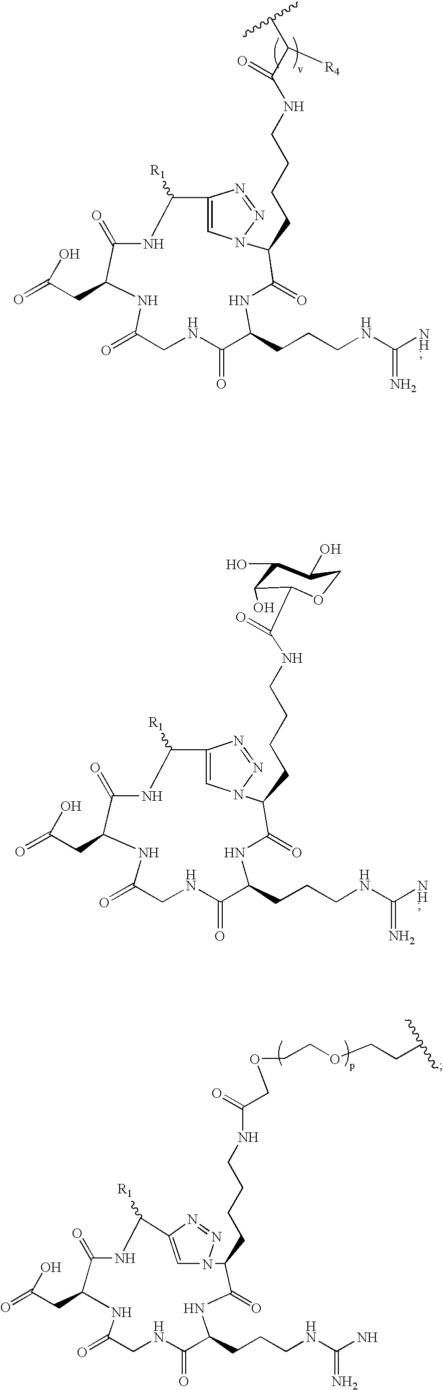

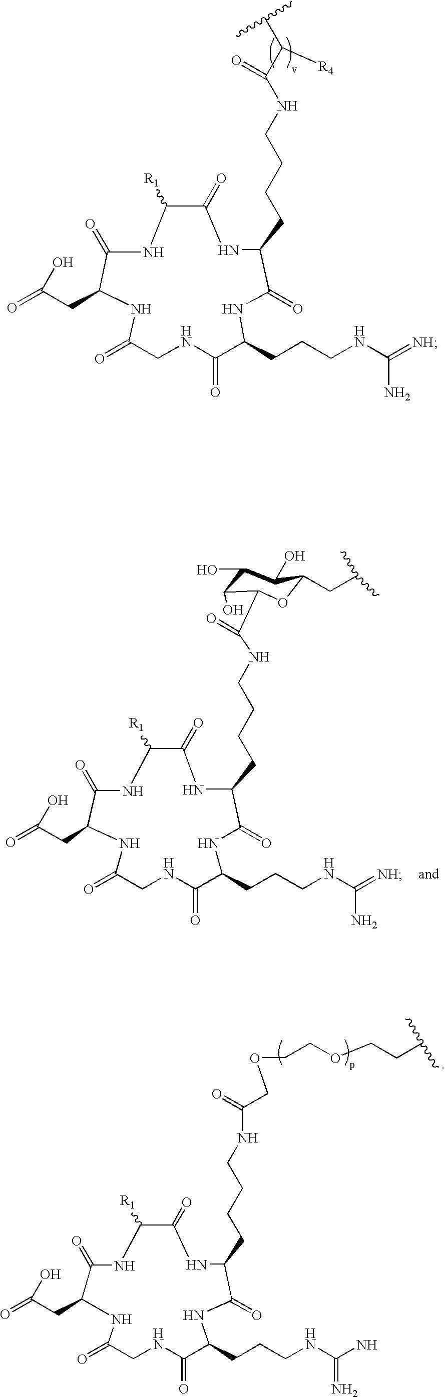

where R₁ is selected from the group consisting of a side chain of a natural amino acid and a side chain of an unnatural amino acid, wherein the natural amino acid and the unnatural amino acid is either in the D or L form; each v is 0, 1, 2, 3, or 4; m is 0, 1, 2, 3 or 4; p is an integer between 1 and 110; q is 1, 2, 3 or 4; r is 1, 2 or 3; r′ is 0 or 1; and s is 1, 2, 3 or 4; wherein the configuration of the chiral centers may be R or S or mixtures thereof.

In yet another embodiment, A is selected from the group consisting of:

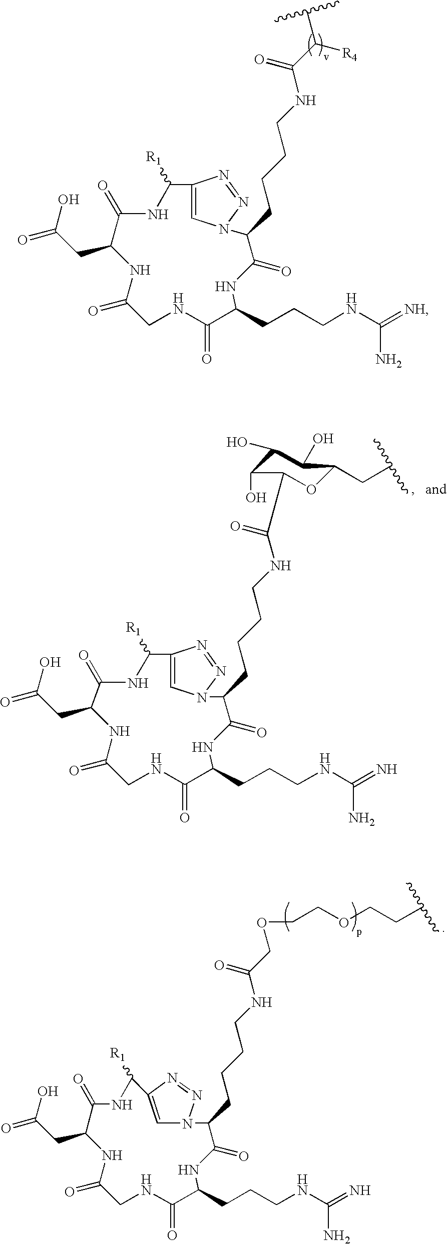

In an alternate embodiment, A is selected from the group consisting of:

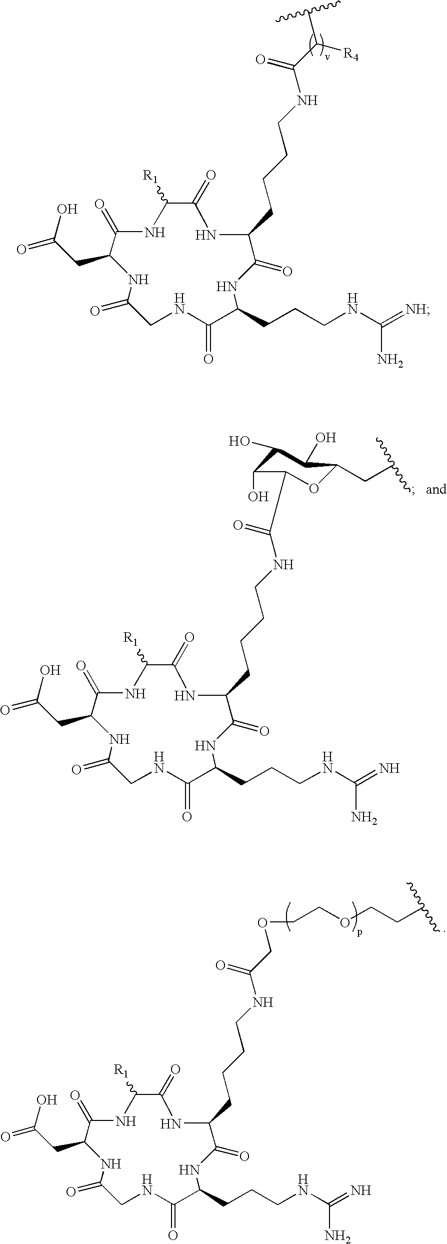

In yet another embodiment, R₁ is a side chain of a natural amino acid; W is

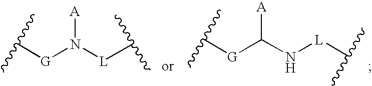

V is 1,2,3-triazolyl; and R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I and ¹³¹I.

In still another embodiment, W is

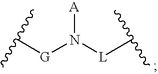

where G is

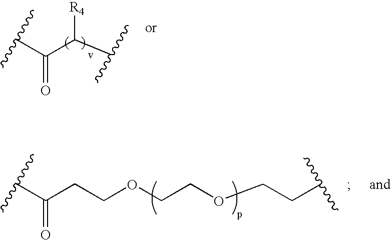

### L is

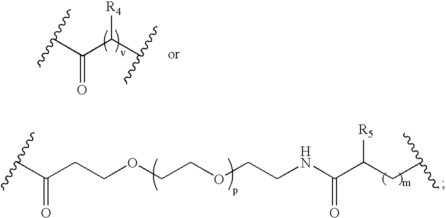

where m is 0 or 1; p is an integer between 1 and 25; v is 0, 1, or 2.

In another embodiment of any of the aspects disclosed herein, W is

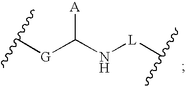

where G is

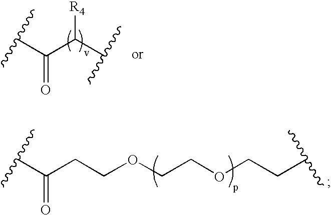

### and L is

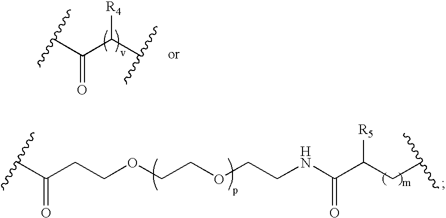

where m is 0 or 1; p is an integer between 1 and 25; v is 0, 1, or 2.

In one variation of any of the embodiments or aspects disclosed herein, G is

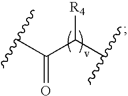

### and A is

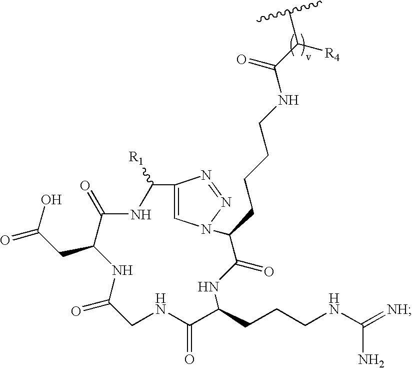

where each R₄ is independently selected from the group consisting of —H and optionally substituted C₁-C₆ alkyl; and each v is 1 or 2. In another variation, G is

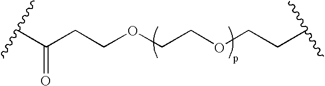

### and A is

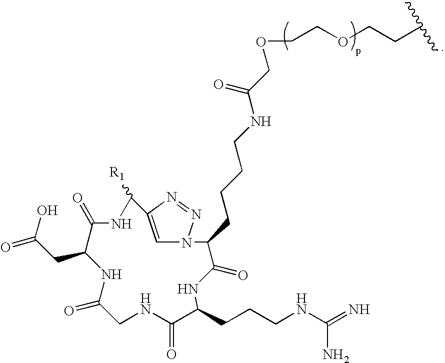

In yet another variation, G is

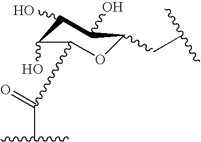

### and A is

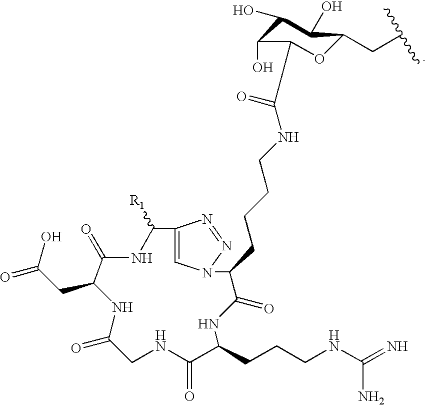

One aspect of the present application is a peptidomimetic of formula II:

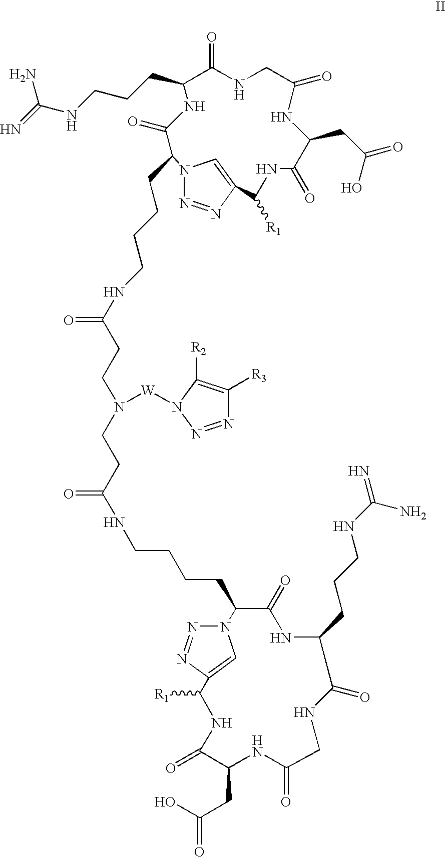

wherein each R₁ is independently selected from the group consisting of a side chain of a natural amino acid and a side chain of an unnatural amino acid, wherein the natural amino acid and the unnatural amino acid is either in the D or L form;

R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I and ¹³¹I;

W is selected from the group consisting of:

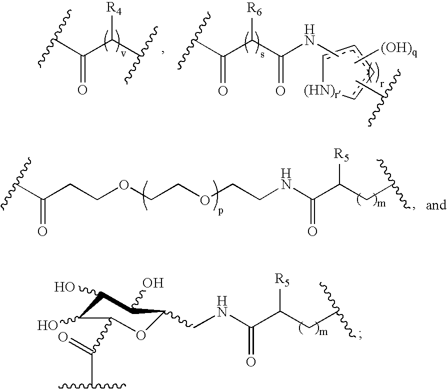

where p is 0 to 15; v is 0, 1, 2, or 3; m is 0, 1 or 2; q is 1 or 2; r is 1,2 or 3; r′ is 0 or 1; and s is 1, 2, 3 or 4; each R₄ and R₅ is independently selected from the group consisting of —H, and optionally substituted C₁-C₆ alkyl; each R₆ is independently selected from the group consisting of —H, —OH, and optionally substituted C₁-C₆ alkyl; wherein the configuration of the chiral center that carries the R₅ substituent may be R or S or mixtures thereof.

In one variation of any of the disclosed embodiments or aspects, W is

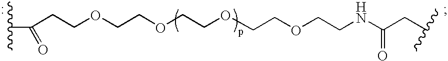

R₃ is —(CH₂)ₙ—¹⁸F; and R₂ is H; where p is 0, 1, 2, 3, 4, or 5; and n is 1, 2, 3, 4, or 5. In another embodiment, p is 0 and n is 3. In another variation, W is

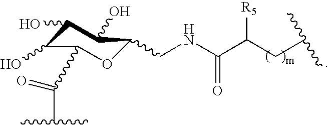

In another varation, W is

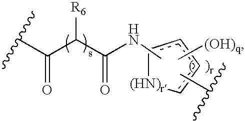

In yet another variation, W is

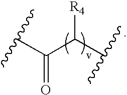

Another aspect of the present application is a peptidomimetic of formula III:

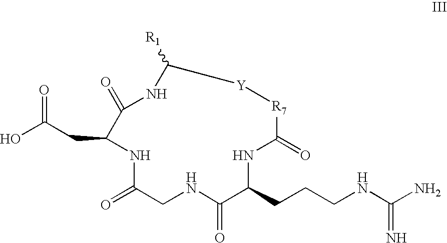

wherein Y is a 5 or 6 membered heterocycle; R₇ is selected from the group consisting of —C(H)(R₁)—, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene-, carbocycle and heterocycle groups are each optionally substituted; and each R₁ is independently selected from the group consisting of a side chain of a natural amino acid and a side chain of an unnatural amino acid, wherein the natural amino acid and the unnatural amino acid is either in the D or L form. In one embodiment, Y is 1,2,3-triazolyl; R₇ is —C(H)(R₁)—; and each R₁ is independently selected from the group consisting of side chains of natural amino acids. In one embodiment, Y is 1,2,3-triazolyl; R₁ is benzyl; R₇ is —C(H)(R₁)—.

In one embodiment, Y is 1,2,3-triazolyl; R₇ is —C(H)[(CH₂)₄)NH₂]— and R₁ is a side chain of a natural amino acid.

In another embodiment, the peptidomimetic is of formula IIIB:

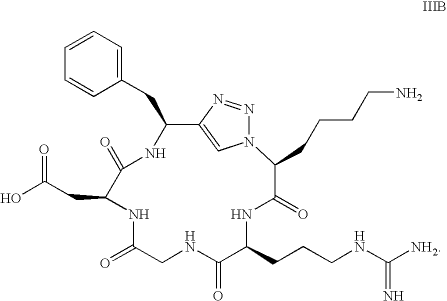

Another aspect of the present application is a peptidomimetic of formula IV:

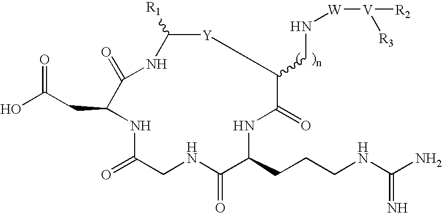

wherein n is 0, 1, 2, 3, or 4; R₁ is a selected from the group consisting of a side chain of natural amino acids and unnatural amino acids, wherein the natural amino acids and unnatural amino acids are either in the D or L form; Y and V is each independently selected from a group consisting of 5 membered heterocycles and 6 membered heterocycles; W is a linker comprising a hydrophilic moiety selected from the group consisting of hydroxyl, carbonyl, sulfonamide, sulfonate, phosphate, polar amino acid moiety, PEG moiety, sugar mimetic, and sugar moiety; R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene, carbocycle and heterocycle groups are optionally substituted; wherein R₂ and R₃ are not both H; wherein the configuration of the chiral centers may be R or S or mixtures thereof; and either R₂ or R₃, or both R₂ and R₃ comprise a radionuclide selected from the group consisting of positron or gamma emitters.

In one embodiment of any aspect or embodiment of the application disclosed herein, V is 1,2,3-triazolyl and n is 4. In another embodiment, R₁ is a side chain of a natural amino acid; V is

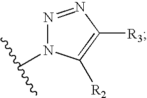

and W is selected from the group consisting of:

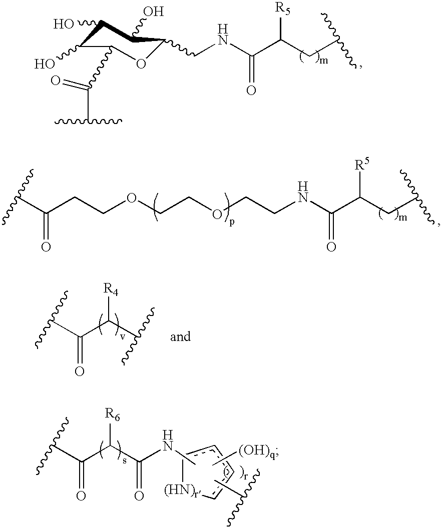

where each R₄ independently selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, C₁-C₆-alkoxy -C₁-C₆-alkyl, and a PEG moiety, wherein the alkyl, alkenyl, alkynyl, alkyloxy, aryl, aryl-alkylene, carbocycle and heterocycle groups are each optionally substituted; wherein the configuration of the chiral centers may be R or S or mixtures thereof;

R₅ is selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, and C₁-C₆-alkoxy-C₁-C₆-alkyl, wherein the alkyl, alkenyl, alkynyl, alkyloxy, aryl, carbocycle, and heterocycle, groups are each optionally substituted;

each R₆ is independently selected from the group consisting of —H, —OH, C₁-C₆ alkyl, C₂-C₆ alkenyl, C₂-C₆ alkynyl, C₁-C₆ alkyloxy, aryl-(C₁-C₆ alkylene)-, hydroxy-C₁-C₆-alkyl, and C₁-C₆-alkoxy-C₁-C₆-alkyl, wherein the alkyl, alkenyl, alkynyl, alkyloxy, and aryl-alkylene groups are each optionally substituted;

q is 1, 2, 3 or 4; r is 1, 2 or 3; r′ is 0 or 1; and s is 1, 2, 3 or 4; v is 0, 1, 2, 3, or 4; m is 0, 1, 2, 3, or 4; and p is an integer between 0 and 15; wherein either R₂ or R₃, or both R₂ and R₃ comprise a radionuclide selected from the group consisting of ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, ¹³¹I, ⁹⁹Tc, ⁷⁵Br, ¹⁵³Gd and ³²P.

In another embodiment of the present application, V is 1,2,3-triazolyl and n is 4; R₁ is a side chain of a natural amino acid; and W is a linker comprising a hydrophilic moiety selected from the group consisting of carbonyl, polar amino acid moiety, PEG moiety, sugar mimetic, and sugar moiety and wherein either R₂ or R₃, or both R₂ and R₃ comprise a radionuclide selected from the group consisting of ¹¹C, ¹³N, ¹⁵O, ¹⁸F, ¹²⁴I, ¹²⁵I, ¹³¹I, and ⁷⁵Br.

In yet another embodiment of any of the aspects or embodiments disclosed herein, W is

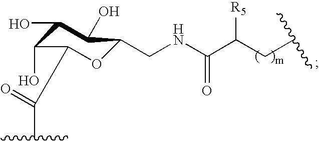

R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I and ¹³¹I; ; R₅ is selected from the group consisting of —H, C₁-C₆ alkyl, C₂-C₆ alkenyl, and C₂-C₆ alkynyl, wherein the alkyl, alkenyl and alkynyl groups are each optionally substituted and wherein the configuration of the chiral center that carries the R₅ substituent may be R or S or mixtures thereof; and m is 0, 1 or 2. In yet another embodiment, R₂ is hydrogen; R₃ is selected from the group consisting of C₁-C₄ alkyl, C₂-C₄ alkenyl, and C₂-C₄ alkynyl, wherein the alkyl, alkenyl and alkynyl groups are each optionally substituted, wherein R₃ comprises a radionuclide selected from the group consisting of ¹¹C, ¹³N, ¹⁵O, and ¹⁸F; R₅ is hydrogen; and m is 0. In still a further embodiment, R₂ is hydrogen; R₃ is an optionally substituted C₁-C₆ alkyl and comprises a radionuclide selected from the group consisting of ¹¹C, ¹³N, ¹⁵O, and ¹⁸F; R₅ is hydrogen; and m is 0 or 1.

In still another embodiment, R₂ and R₃ are each independently selected from the group consisting of —H, C₁-C₆ alkyl, C₂-C₆ alkenyl, C₂-C₆ alkynyl, wherein the alkyl, alkenyl and alkynyl groups are each optionally substituted; wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I, and ¹³¹I;

W is

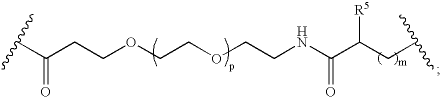

where R₅ is selected from the group consisting of —H, C₁-C₆ alkyl, C₂-C₆ alkenyl, and C₂-C₆ alkynyl, wherein the alkyl, alkenyl and alkynyl groups are each optionally substituted and wherein the configuration of the chiral center that carries the R₅ substituent may be R or S or mixtures thereof; m is 0, 1, or 2; and p is an integer between 1 and 90. In another embodiment or aspect of the application, R₂ is hydrogen; R₃ is selected from the group consisting of C₁-C₄ alkyl, C₂-C₄ alkenyl, and C₂-C₄ alkynyl, wherein the alkyl, alkenyl and alkynyl groups are each optionally substituted, and R₃ comprises a radionuclide selected from the group consisting of ¹¹C, ¹³N, ¹⁵O, and ¹⁸F; R₅ is hydrogen; m is 0; and p is an integer between 1 and 15.

In one embodiment of any of the disclosed aspects of the present application,

W is

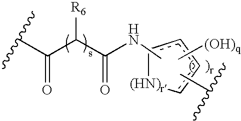

where each R₆ is independently selected from the group consisting of —H, —OH, C₁-C₆ alkyl, C₂-C₆ alkenyl, C₁-C₆ alkyloxy, hydroxy-C₁-C₆-alkyl, and C₁-C₆-alkoxy-C₁-C₆-alkyl, wherein the alkyl, alkenyl, and alkyloxy groups are each optionally substituted; q is 2, 3 or 4; r is 1, 2 or 3; r′ is 0 or 1; and s is 1 or 2. In one embodiment, each R₆ is independently selected from the group consisting of —H, —OH and optionally substituted C₁-C₆ alkyl; q is 2; r is 2 or 3; and r′ is 0. In another embodiment, each R₆ is independently selected from the group consisting of —H, —OH and optionally substituted C₁-C₆ alkyl, r′ is 1, r is 1 or 2, q is 1 or 2.

In another embodiment, W is

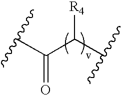

where each R₄ is independently selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, C₁-C₆-alkoxy-C₁-C₆-alkyl, and a PEG moiety, wherein the alkyl, alkenyl, alkynyl, alkyloxy, aryl, aryl-alkylene, carbocycle and heterocycle groups are each optionally substituted; and v is 1, 2, 3, or 4. In one variation, each R₄ is independently selected from the group consisting of —H, C₁-C₆ alkyl, C₂-C₆ alkenyl, C₁-C₆ alkyloxy, hydroxy-C₁-C₆-alkyl, C₁-C₆-alkoxy-C₁-C₆-alkyl, and a PEG moiety, wherein the alkyl, alkenyl, and alkyloxy groups are each optionally substituted.

Another aspect of the present application is the cyclic peptidomimetic

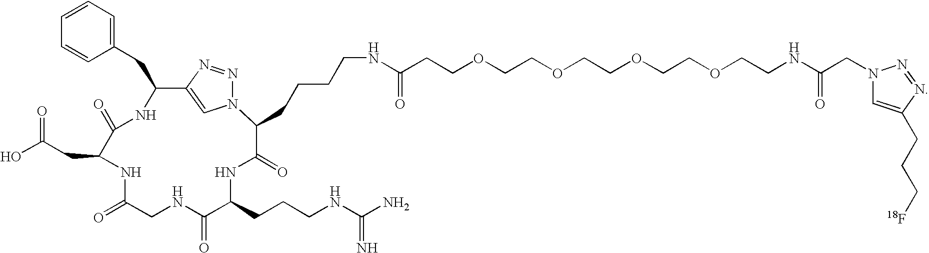

Yet another aspect of the present application is a cyclic peptidomimetic selected from the group consisting of:

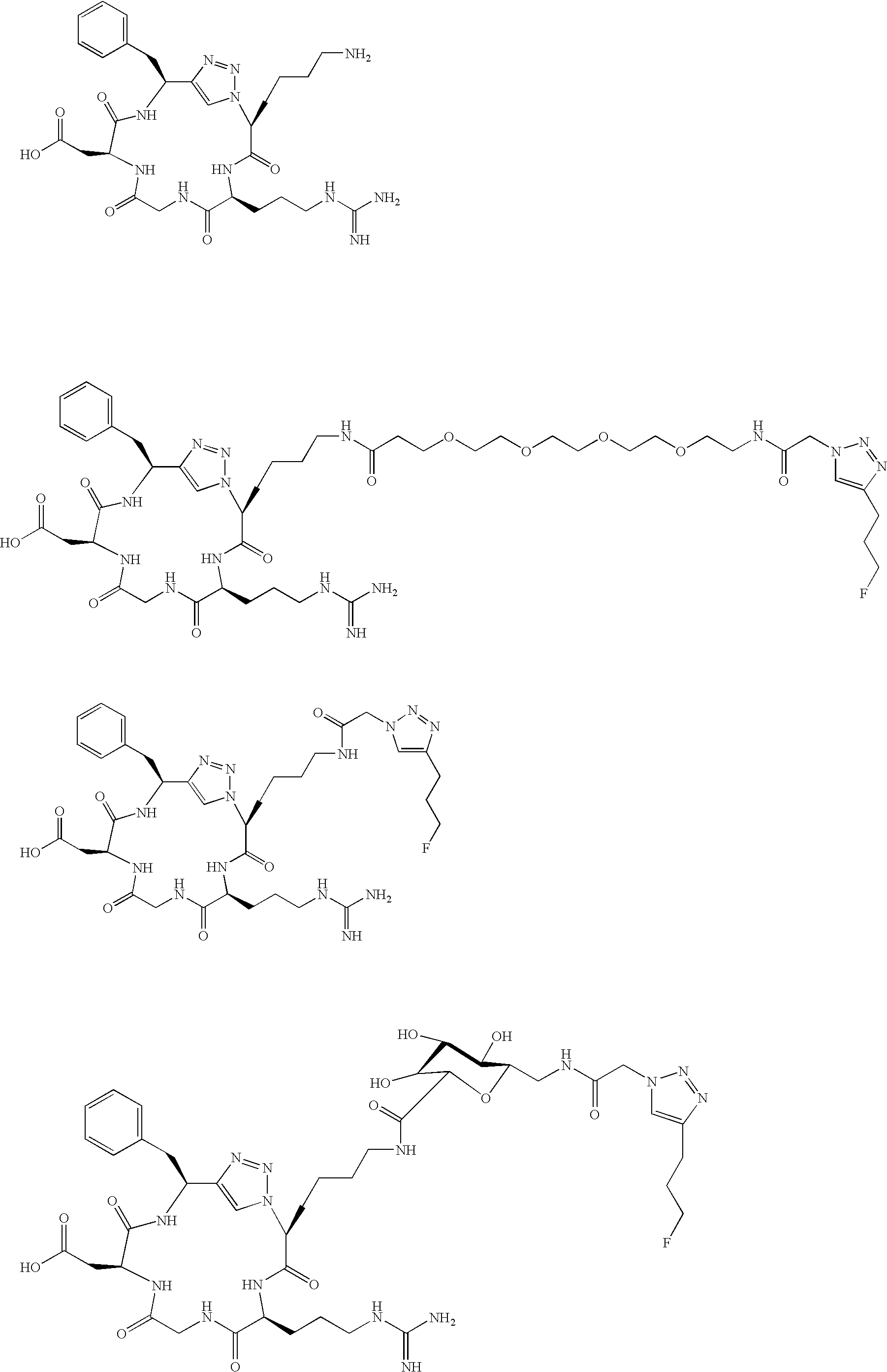

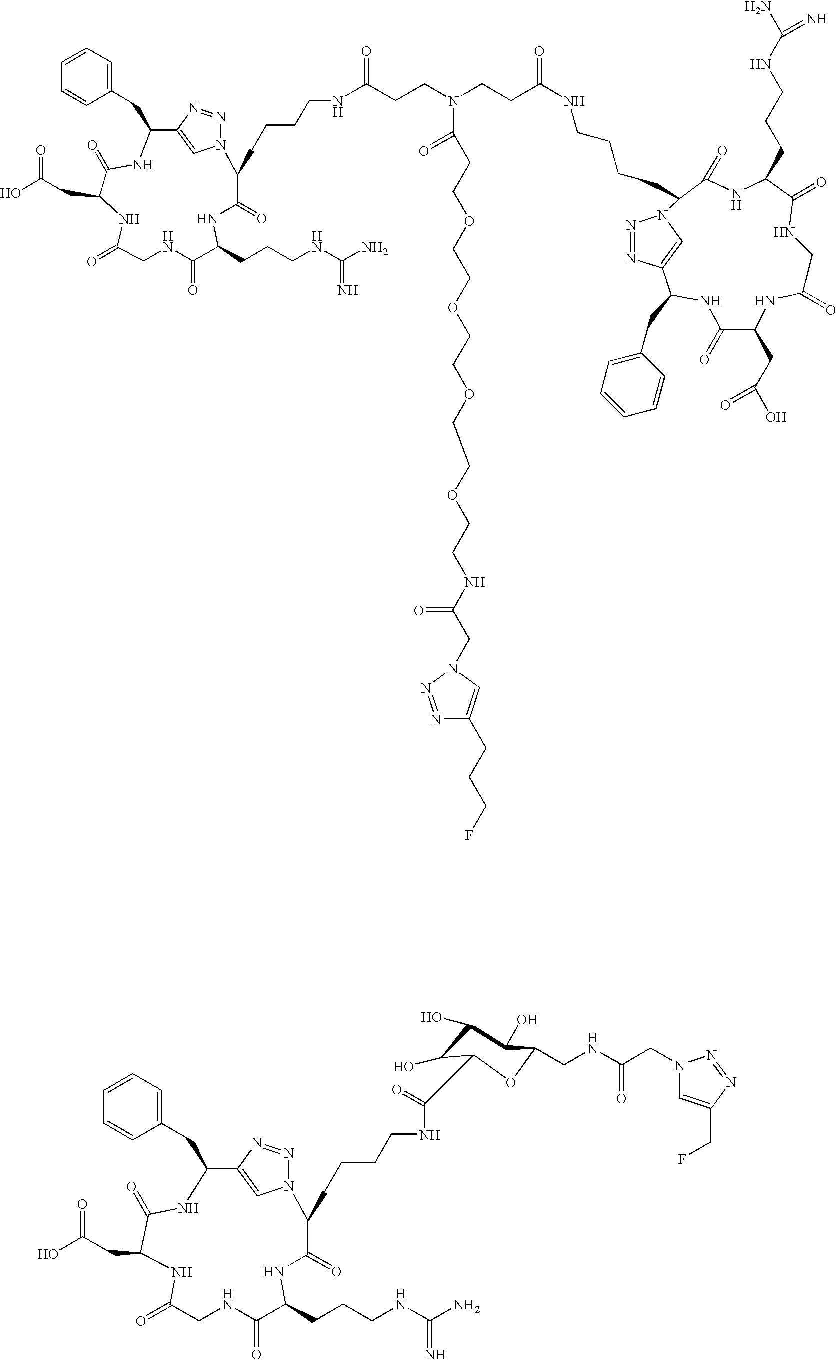

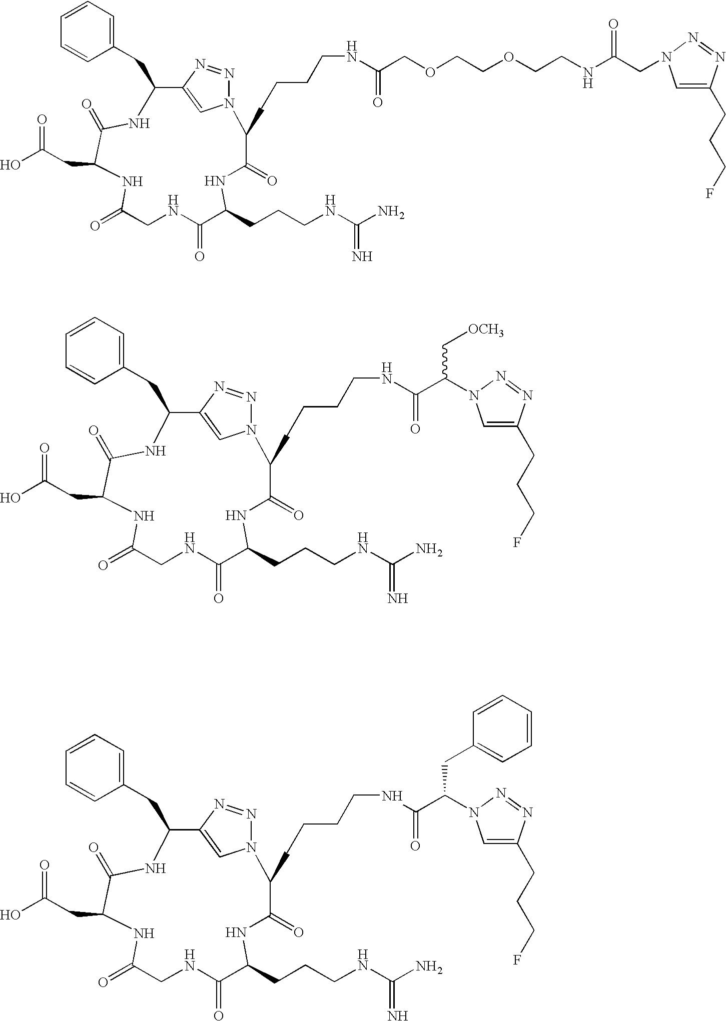

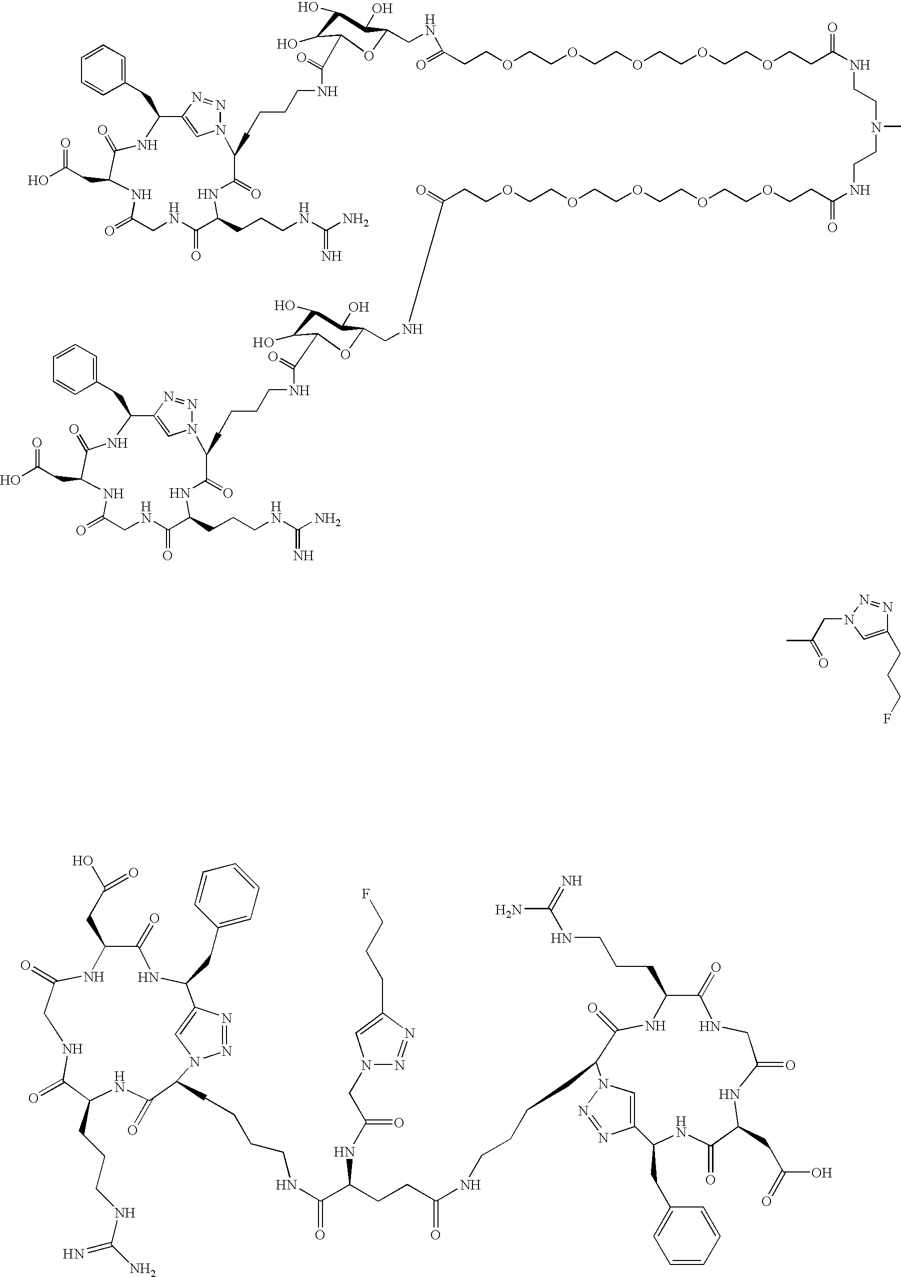

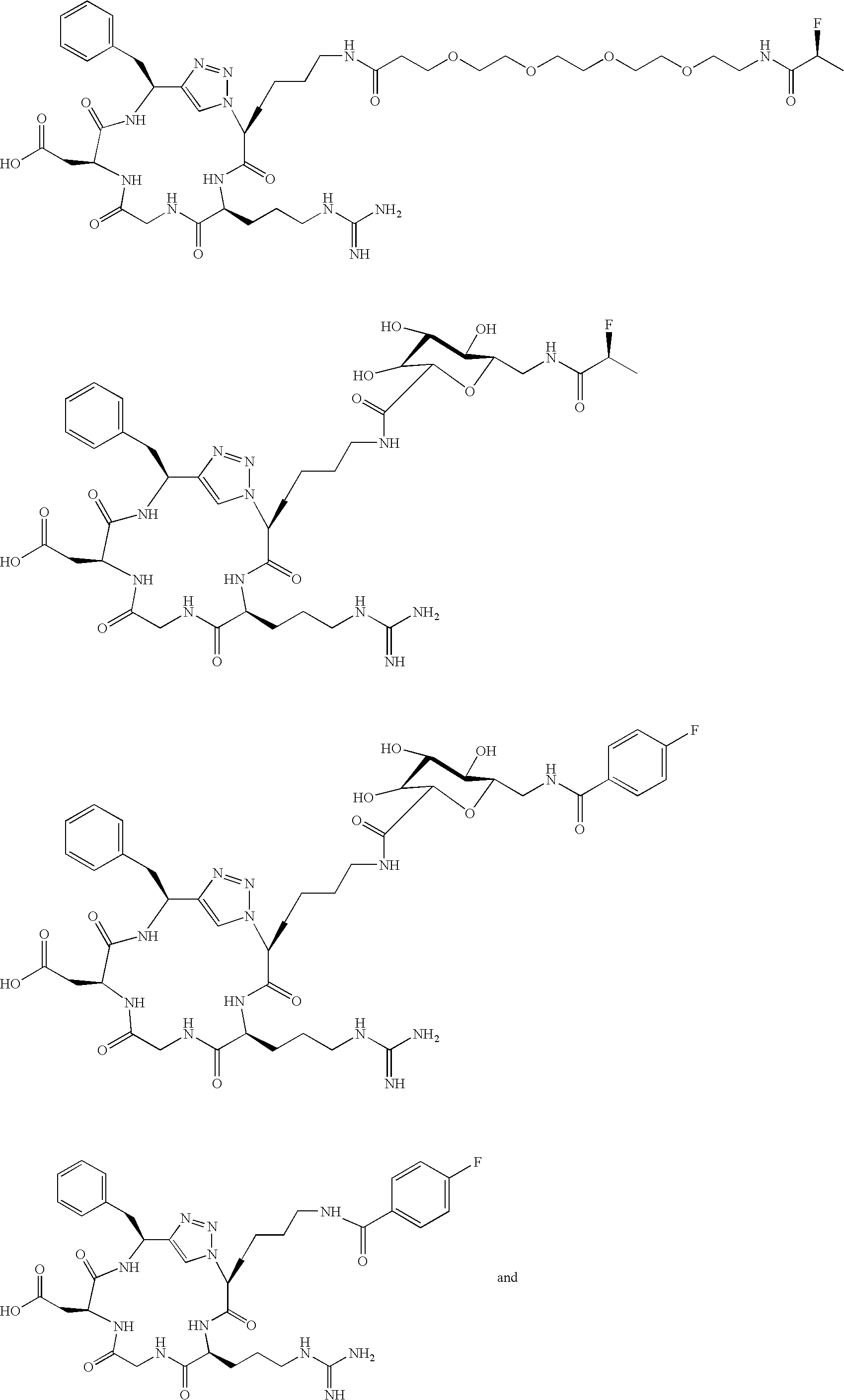

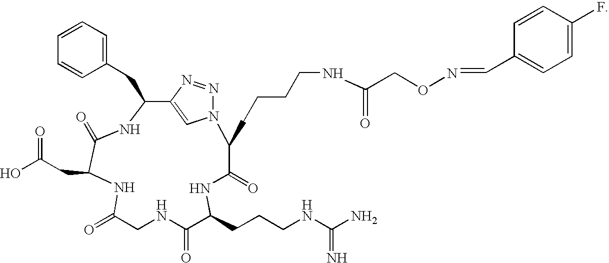

One aspect of the present application is a pharmaceutical composition comprising a radiolabeled cyclic peptidomimetic of formula I:

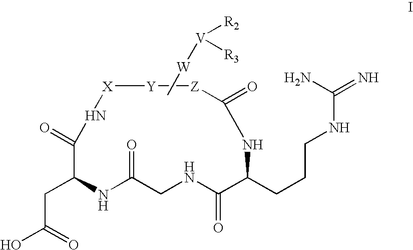

wherein W is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;

V is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;

wherein at least one, but not both of W and V is a 5- or 6-membered heterocycle;

X is selected from the group consisting of —C₁-C₆ alkyl-(5- to 6-membered heterocycle)-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

Y is selected from the group consisting of 5- or 6-membered heterocycle, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

Z is selected from the group consisting of -(5- or 6-membered heterocycle)-C₁-C₆ alkyl-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

any one of X, Y, or Z but not more than one of X, Y and Z is a 5- or 6-membered heterocycle;

where each R₁ is independently selected from the group consisting of a side chain of a natural amino acid and a side chain of an unnatural amino acid, wherein the natural amino acid and the unnatural amino acid is either in the D or L form;

R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene, carbocycle and heterocycle groups are each optionally substituted; and optionally the fragment W—V(R₂)(R₃) is absent; wherein at least one of W, X, Y, Z, R₂, and R₃ comprises a radionuclide selected from the group consisting of positron or gamma emitters; and a pharmaceutically acceptable carrier.

Another aspect of the present application is a pharmaceutical composition comprising a radiolabeled cyclic peptidomimetic of formula II or formula IV:

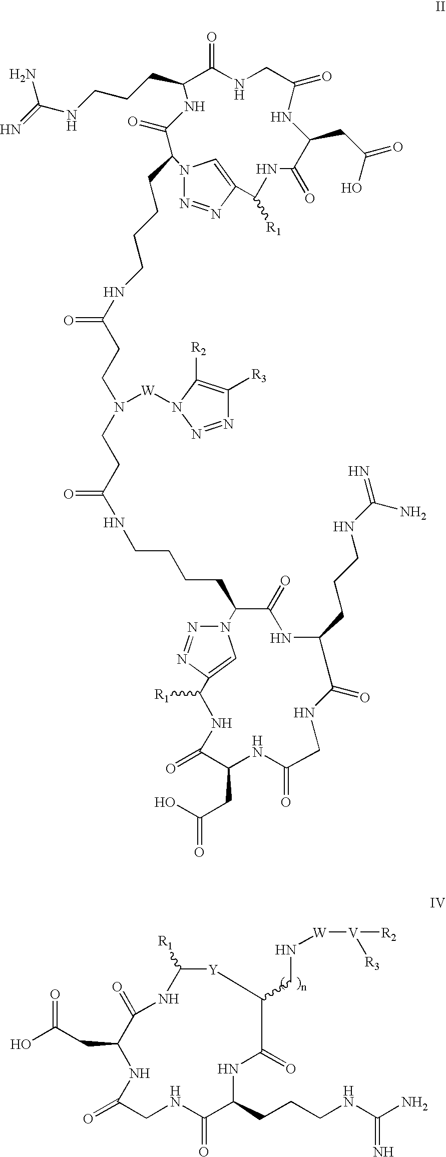

wherein V is 1,2,3-triazolyl; n is 1, 2, 3, 4 or 5;

each R₁ is independently selected from the group consisting of a side chain of a natural amino acid and a side chain of an unnatural amino acid, wherein the natural amino acid and the unnatural amino acid is either in the D or L form;

R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I and ^{13l}I;

W is selected from the group consisting of

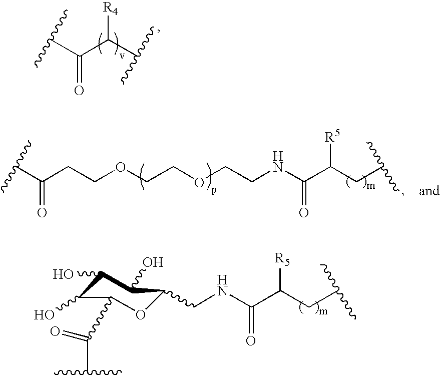

where p is 0 to 15; v is 0, 1, 2, or 3; m is 0, 1 or 2; each R₄ and R₅ is independently selected from the group consisting of —H, and optionally substituted C₁-C₆ alkyl; wherein the configuration of the chiral center that carries the R₅ substituent may be R or S or mixtures thereof; and a pharmaceutically acceptable carrier.

Another aspect of the present application is a pharmaceutical composition comprising a radiolabeled cyclic peptidomimetic selected from the group consisting of:

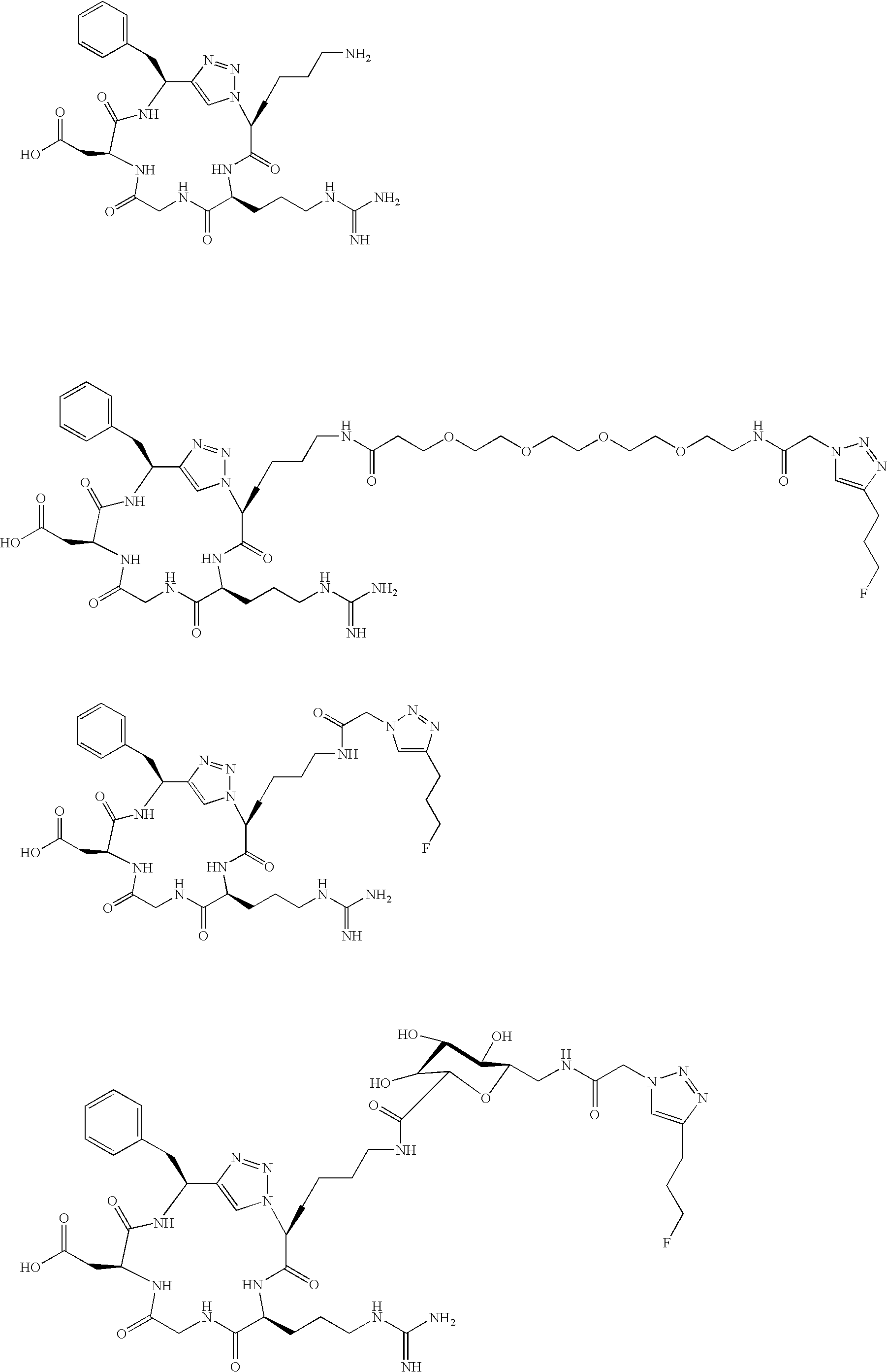

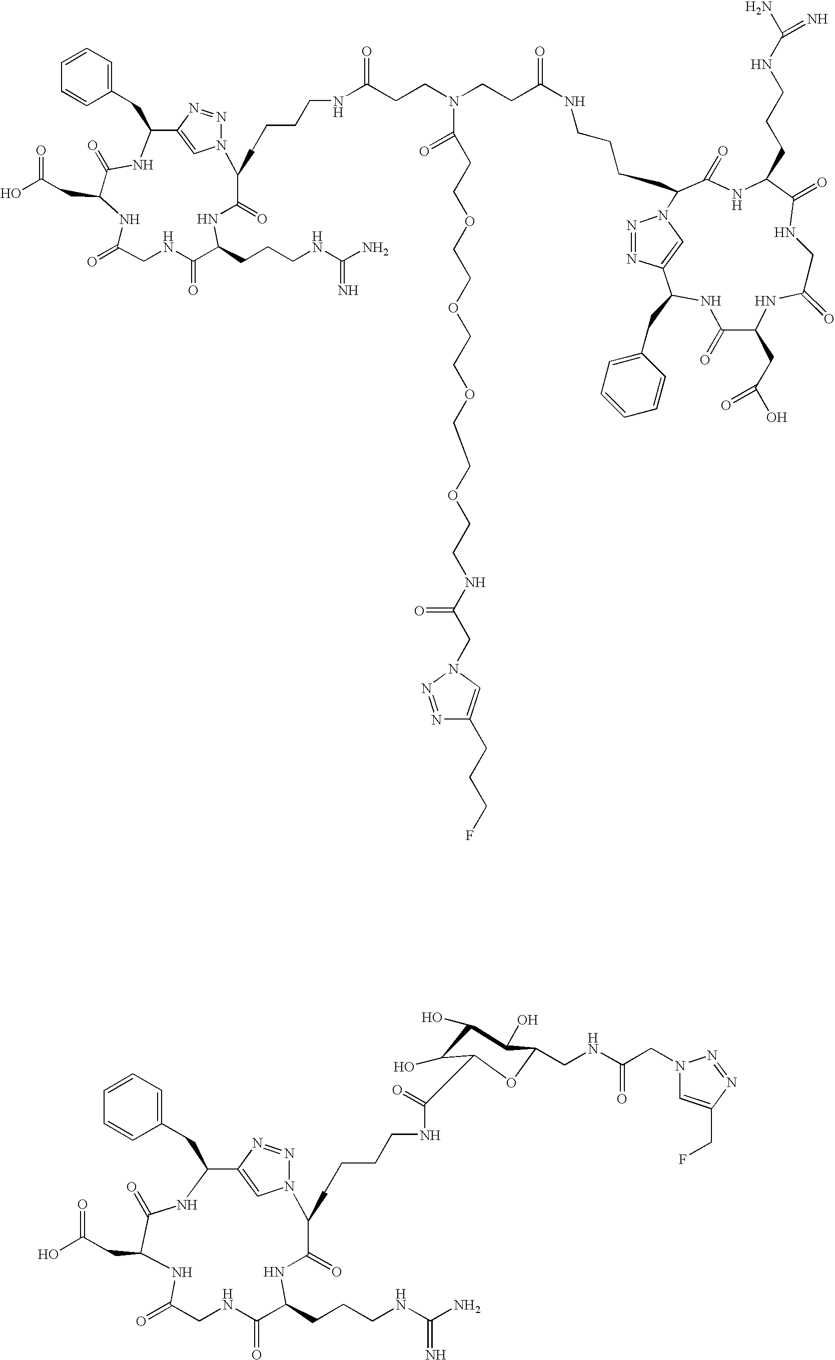

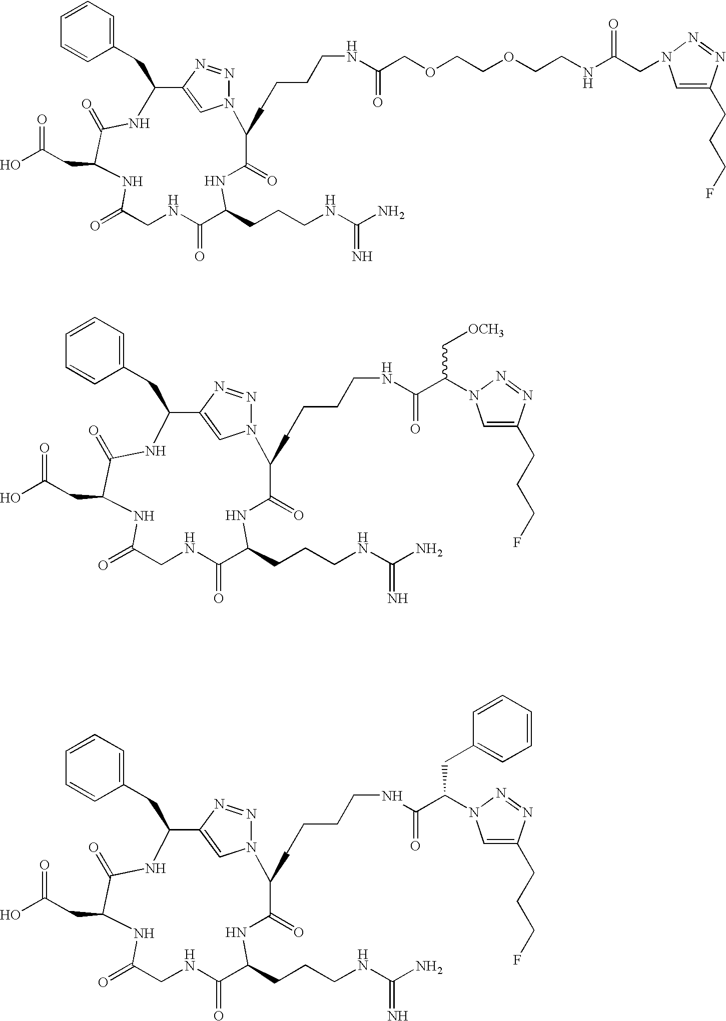

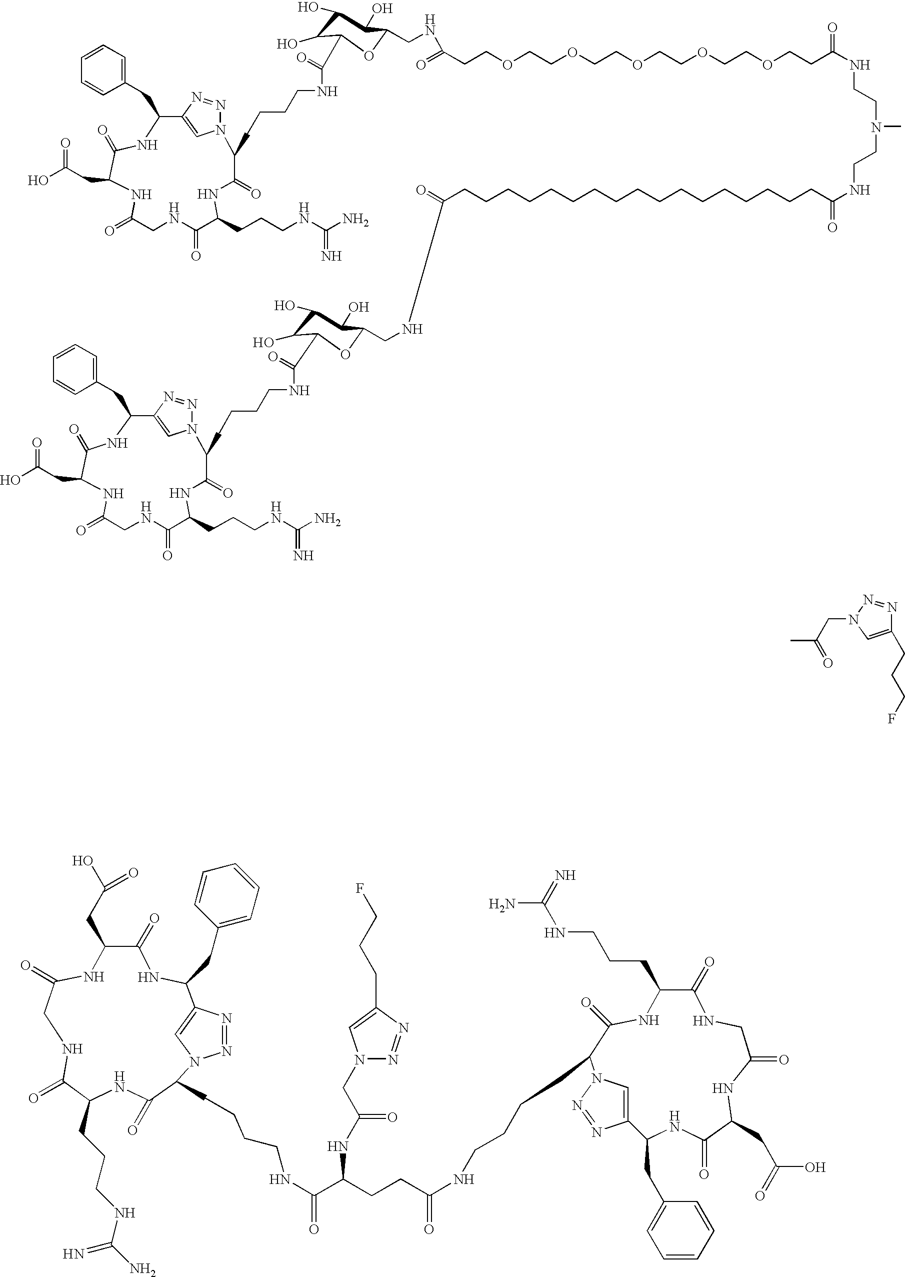

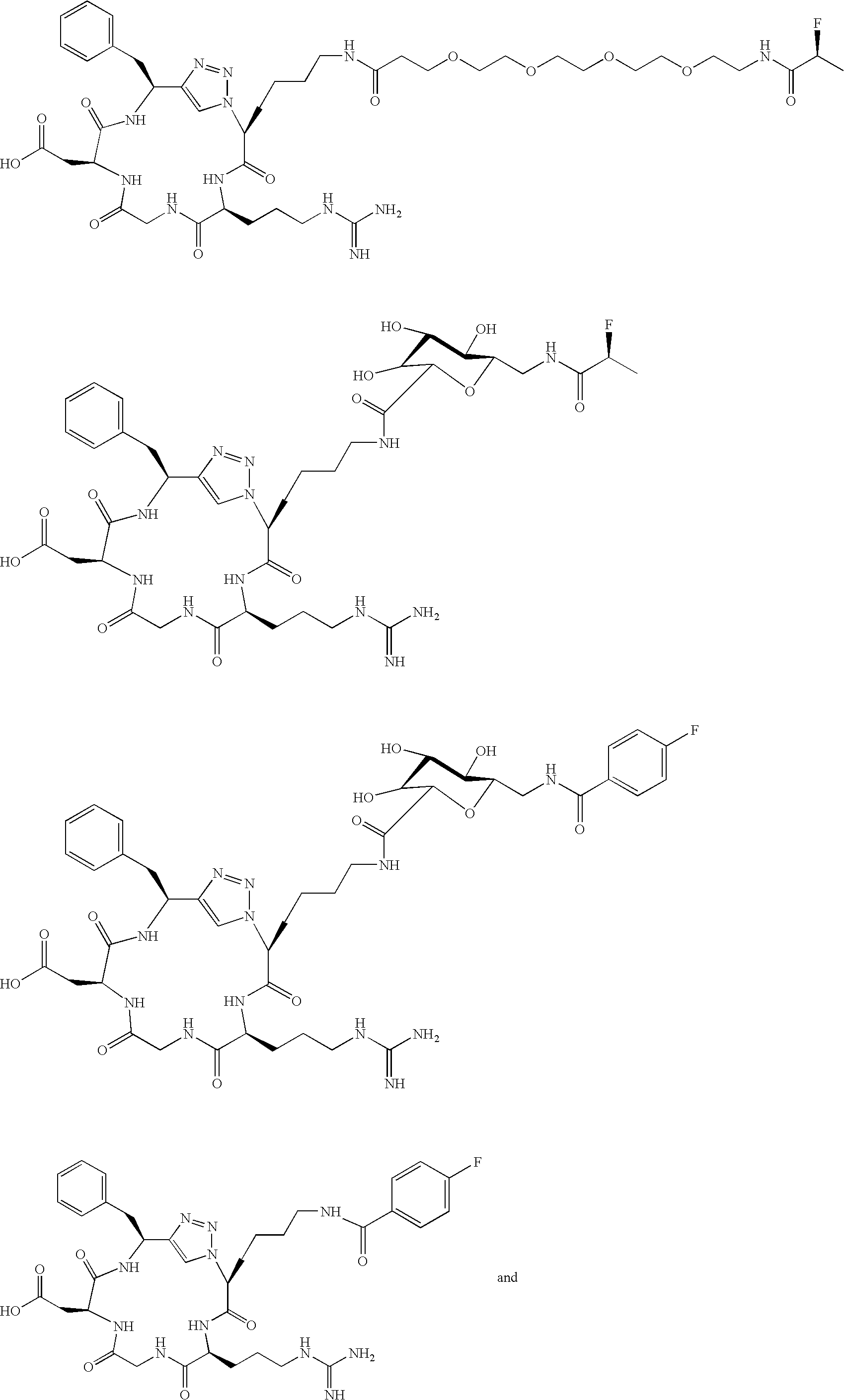

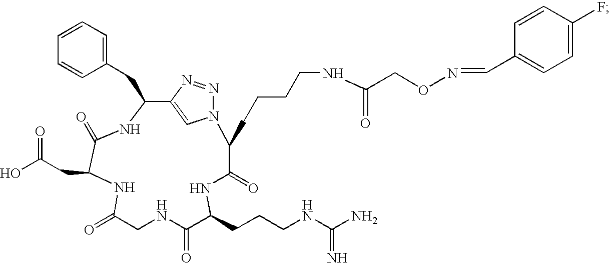

and a pharmaceutically acceptable carrier.

Yet another aspect of the present application is a method of monitoring the level of integrin αᵥβ₃ or visualizing integrin αᵥβ₃ expression within a body of a patient, the method comprising: (a) administering to the patient a radiolabeled cyclic peptidomimetic; and (b) employing a nuclear imaging technique selected from the group consisting of positron emission tomography (PET) and single photon emission computed tomography (SPECT) for monitoring or visualizing a distribution of the cyclic peptidomimetic within the body or within a portion thereof; wherein the radiolabeled cyclic peptidomimetic is of formula I:

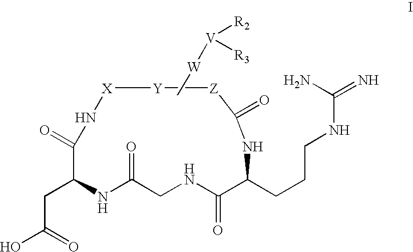

wherein W is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;

V is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;

wherein at least one, but not both of W and V is a 5- or 6-membered heterocycle;

X is selected from the group consisting of —C₁-C₆ alkyl-(5- to 6-membered heterocycle)-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

Y is selected from the group consisting of 5- or-6-membered heterocycle, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

Z is selected from the group consisting of -(5- or 6-membered heterocycle)-C₁-C₆ alkyl-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

any one of X, Y, or Z but not more than one of X, Y and Z is a 5- or 6-membered heterocycle;

where each R₁ is independently selected from the group consisting of a side chain of a natural amino acid and a side chain of an unnatural amino acid, wherein the natural amino acid and the unnatural amino acid is either in the D or L form;

R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene, carbocycle and heterocycle groups are each optionally substituted; and

optionally the fragment W—V(R₂)(R₃) is absent; wherein at least one of W, X, Y, Z, R₂, and R₃ comprises a radionuclide selected from the group consisting of positron or gamma emitters.

Another aspect of the present application is a method of monitoring the level of integrin αᵥβ₃ or visualizing integrin αᵥβ₃ expression within a body of a patient, the method comprising: (a) administering to the patient a radiolabeled cyclic peptidomimetic; and (b) employing a nuclear imaging technique selected from the group consisting of positron emission tomography (PET) and single photon emission computed tomography (SPECT) for monitoring or visualizing a distribution of the radiolabeled cyclic peptidomimetic within the body or within a portion thereof; wherein the radiolabeled cyclic peptidomimetic is of formula II or formula IV:

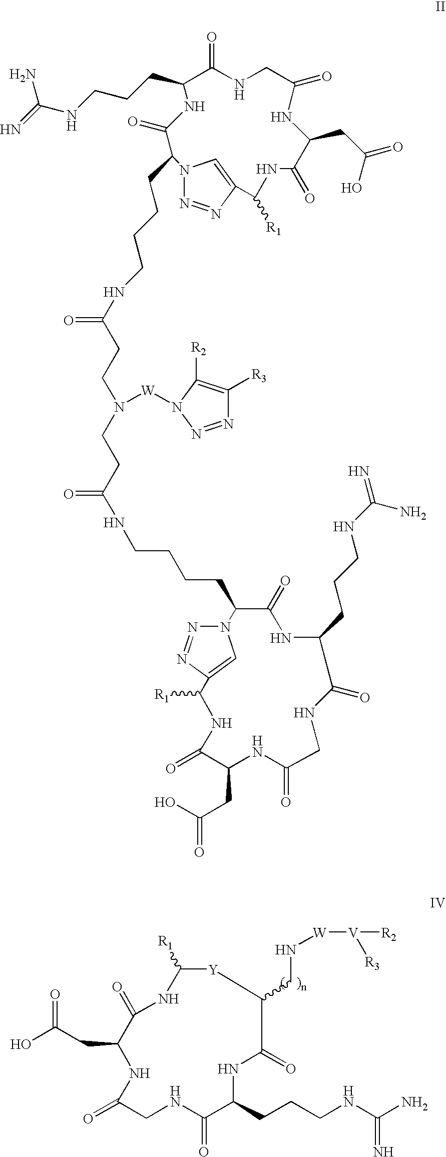

wherein V is 1,2,3-triazolyl;

n is 1, 2, 3, 4 or 5;

each R₁ is independently selected from the group consisting of a side chain of a natural amino acid and a side chain of an unnatural amino acid, wherein the natural amino acid and the unnatural amino acid is either in the D or L form;

R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I and ¹³¹I;

W is selected from the group consisting of

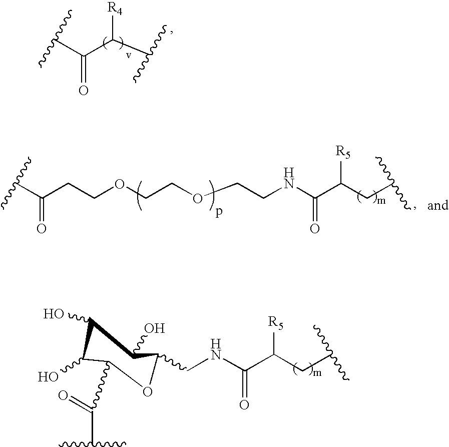

where p is 0 to 15; v is 0, 1, 2, or 3; m is 0, 1 or 2; each R₄ and R₅ is independently selected from the group consisting of —H, and optionally substituted C₁-C₆ alkyl; wherein the configuration of the chiral center that carries the R₅ substituent may be R or S or mixtures thereof.

Yet another aspect of the present application is a method of monitoring the level of integrin αᵥβ₃ or visualizing integrin αᵥβ₃ expression within a body of a patient, the method comprising: (a) administering to the patient a radiolabeled cyclic peptidomimetic; and (b) employing a nuclear imaging technique selected from the group consisting of positron emission tomography (PET) and single photon emission computed tomography (SPECT) for monitoring or visualizing a distribution of the radiolabeled cyclic peptidomimetic within the body or within a portion thereof; wherein the radiolabeled peptidomimetic selected from the group consisting of:

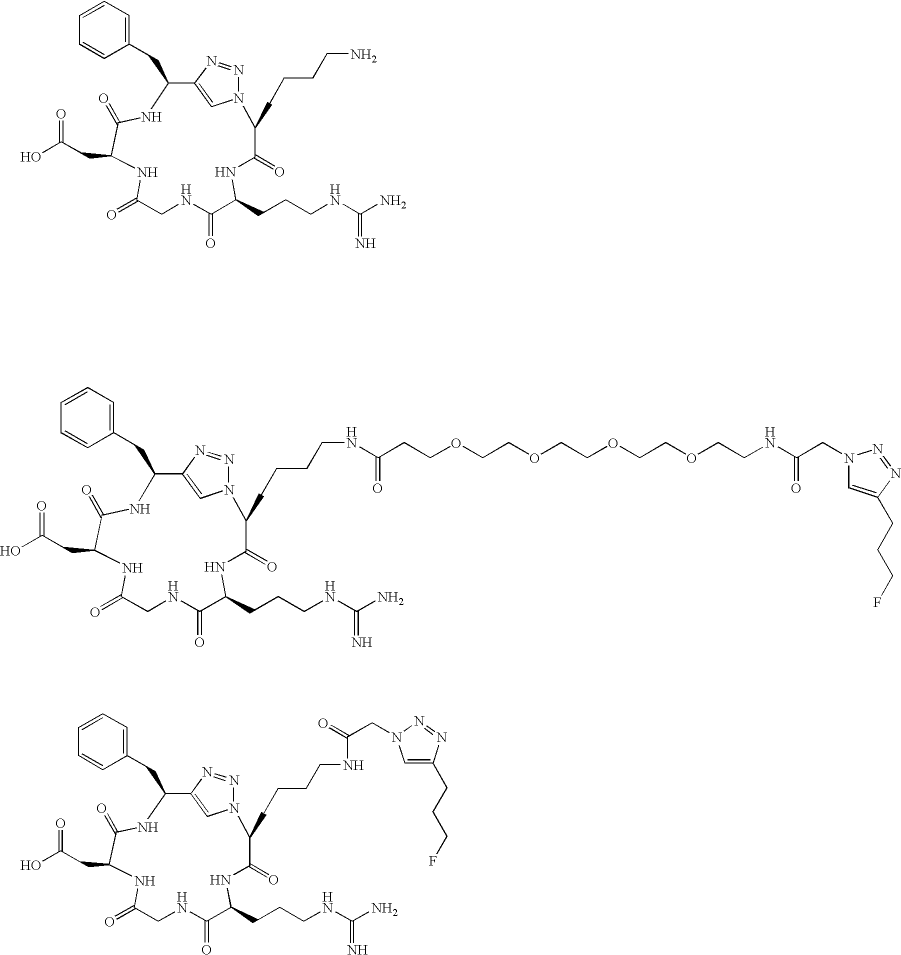

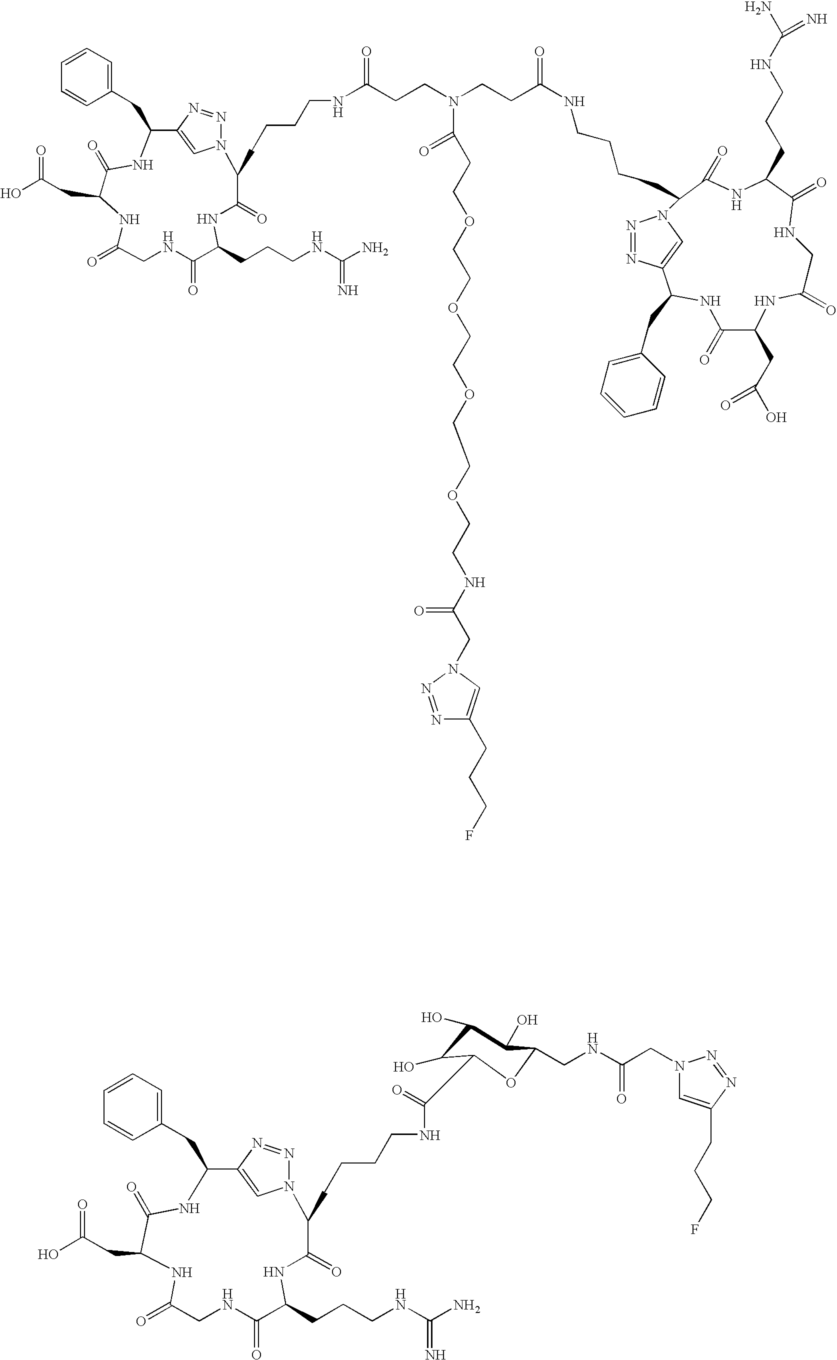

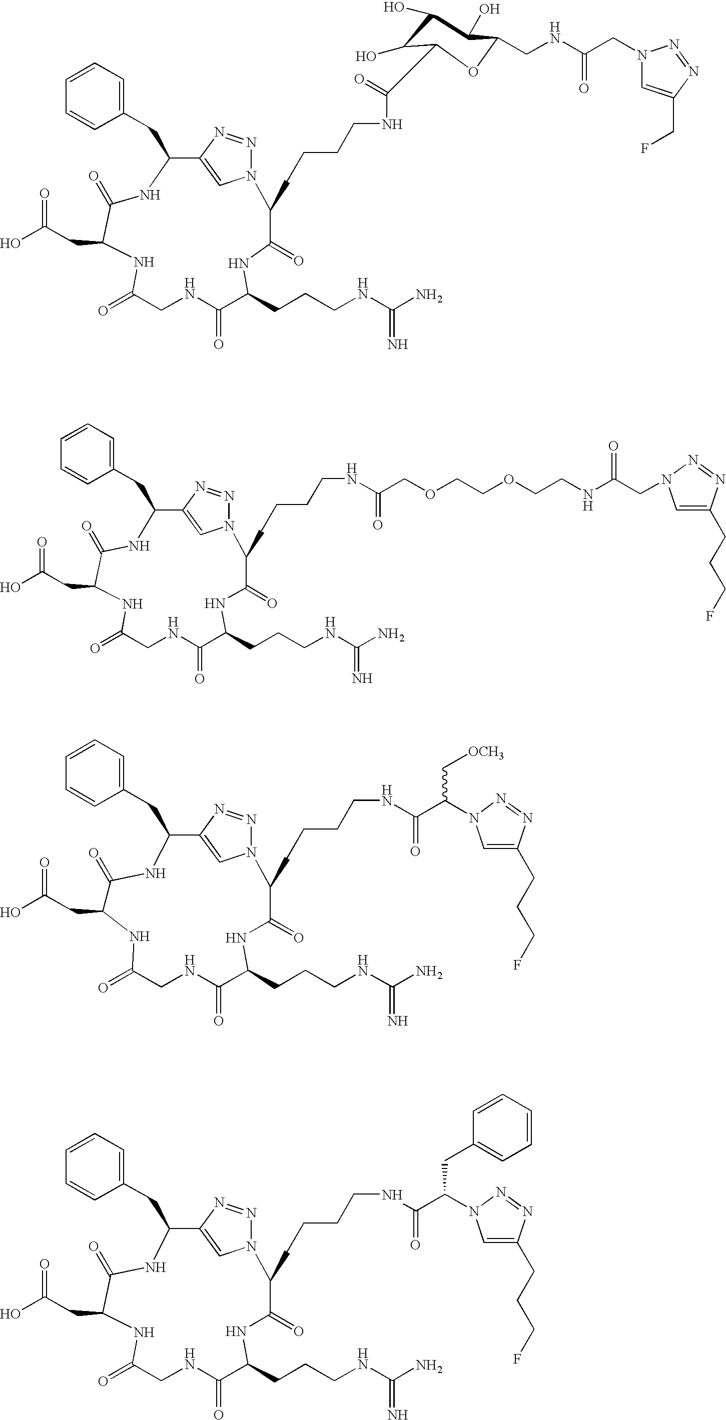

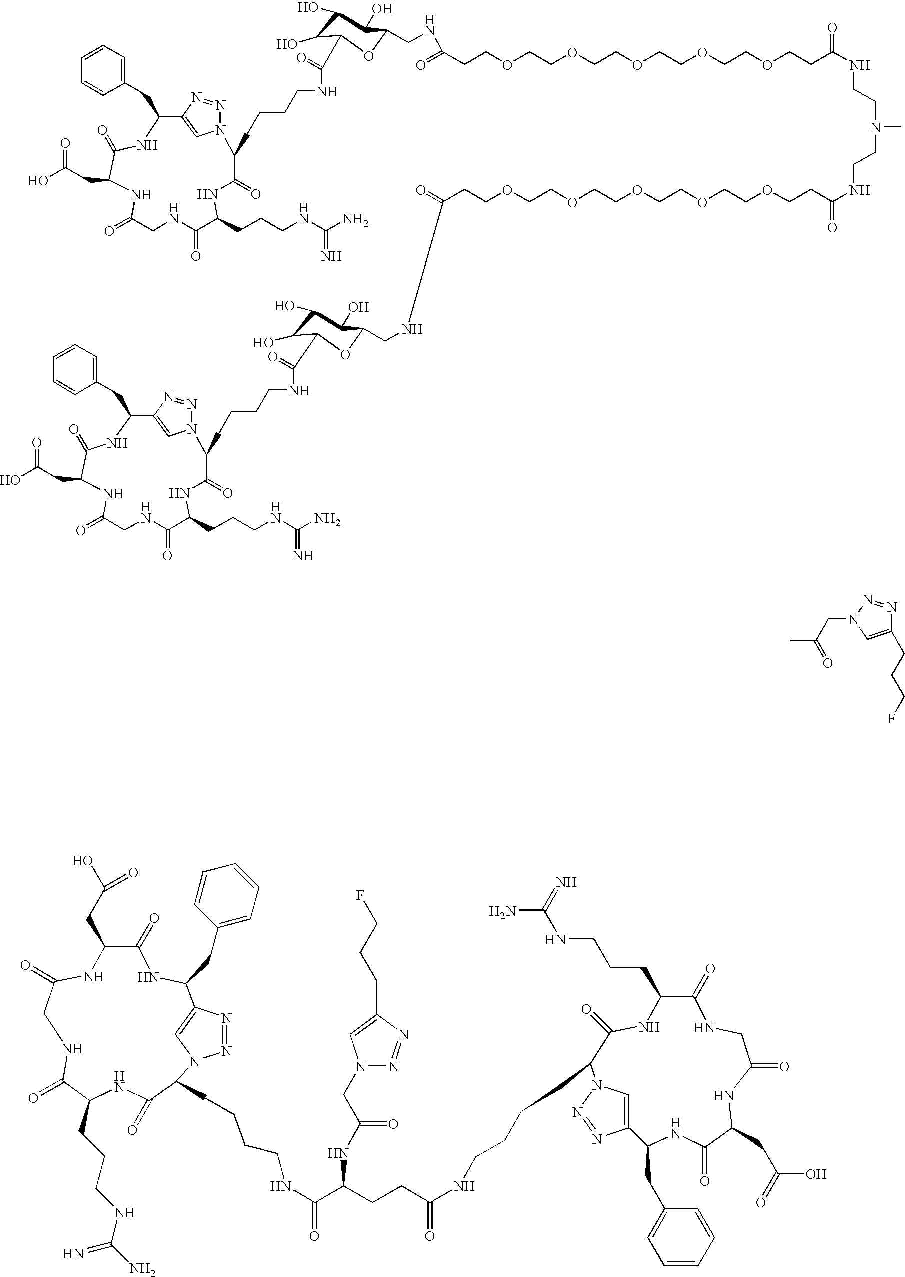

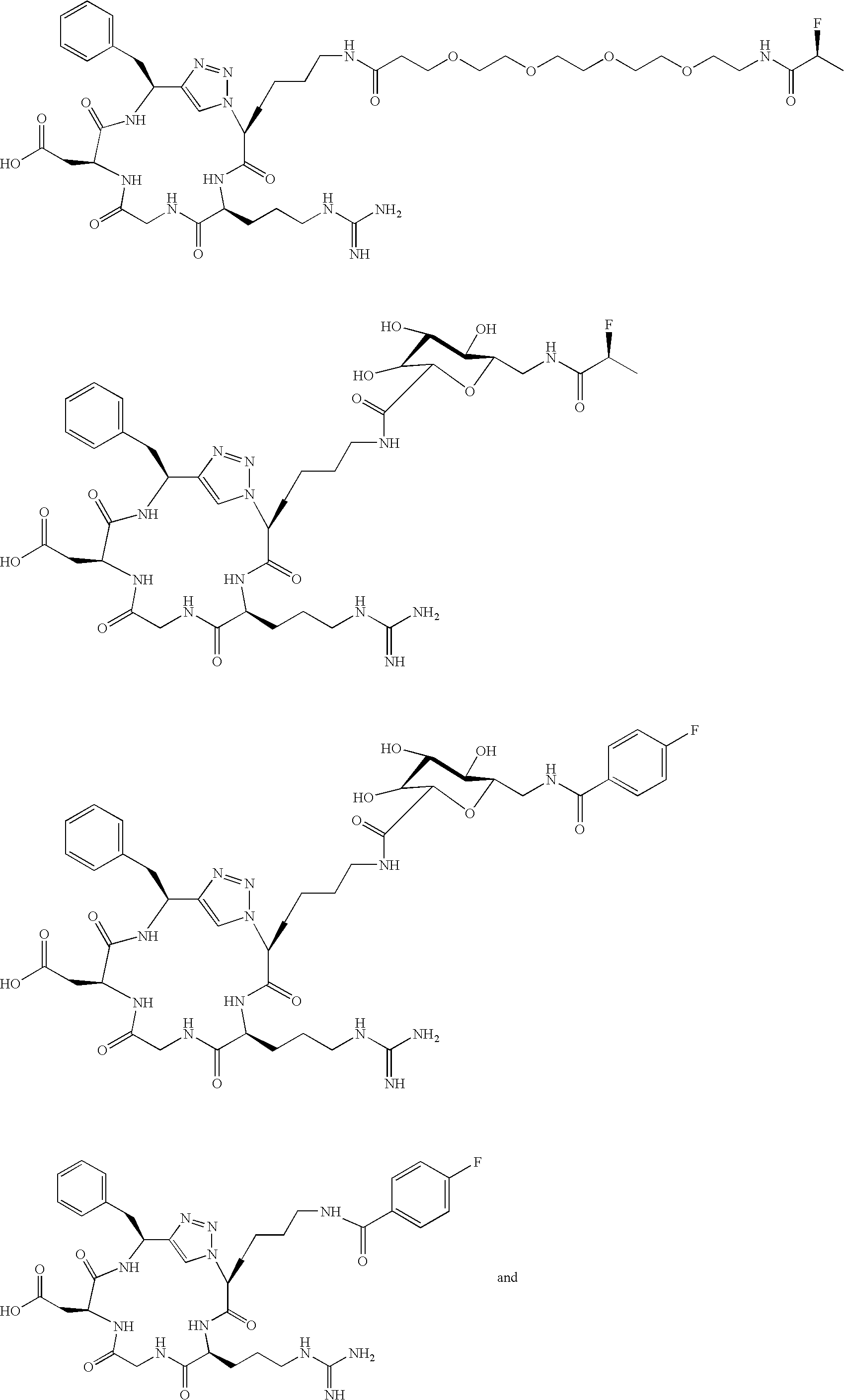

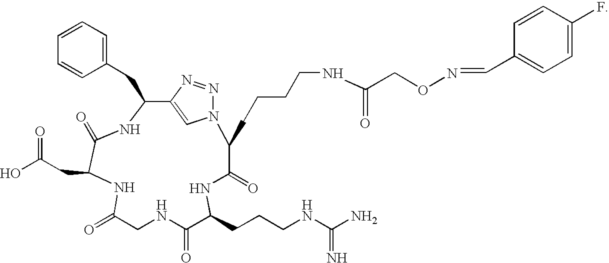

Still another aspect of the present application is a method for imaging of blood vessel growth in solid tumors based on expression of integrin αᵥβ₃ within the body of a patient, the method comprising: (a) administering to the patient radiolabeled cyclic peptidomimetic; (b) employing a nuclear imaging technique selected from the group consisting of positron emission tomography (PET) and single photon emission computed tomography (SPECT) for imaging a distribution of the radiolabeled cyclic peptidomimetic within the body or within a portion thereof; and c) correlating the distribution of the radiolabeled cyclic peptidomimetic to the growth of blood vessels in solid tumors, wherein the radiolabeled cyclic peptidomimetic is of formula I:

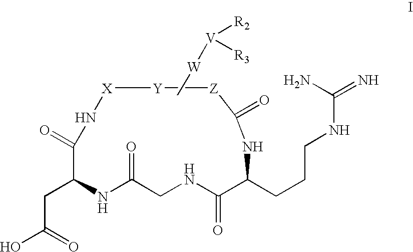

wherein W is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;

V is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;

wherein at least one, but not both of W and V is a 5- or 6-membered heterocycle;

X is selected from the group consisting of —C₁-C₆ alkyl-(5- to 6-membered heterocycle)-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

Y is selected from the group consisting of 5- or 6-membered heterocycle, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

Z is selected from the group consisting of -(5- or 6-membered heterocycle)—C₁-C₆ alkyl-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;

any one of X, Y, or Z but not more than one of X, Y and Z is a 5- or 6-membered heterocycle;

where each R₁ is independently selected from the group consisting of a side chain of a natural amino acid and a side chain of an unnatural amino acid, wherein the natural amino acid and the unnatural amino acid is either in the D or L form;

R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene, carbocycle and heterocycle groups are each optionally substituted; and

optionally the fragment W—V(R₂)(R₃) is absent; wherein at least one of W, X, Y, Z, R₂, and R₃ comprises a radionuclide selected from the group consisting of positron or gamma emitters.

Another aspect of the present application is a method for imaging of blood vessel growth in solid tumors based on expression of integrin αᵥβ₃ within the body of a patient, the method comprising: (a) administering to the patient radiolabeled cyclic peptidomimetic; (b) employing a nuclear imaging technique selected from the group consisting of positron emission tomography (PET) and single photon emission computed tomography (SPECT) for imaging a distribution of the radiolabeled cyclic peptidomimetic within the body or within a portion thereof; and c) correlating the distribution of the radiolabeled cyclic peptidomimetic to the growth of blood vessels in solid tumors, wherein the radiolabeled cyclic peptidomimetic is of formula II or formula IV:

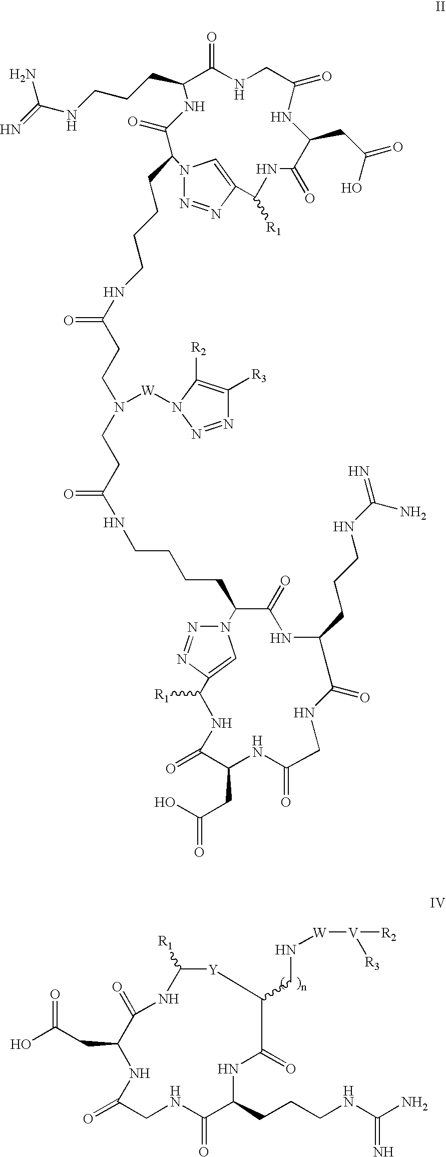

wherein

V is 1,2,3-triazolyl;

n is 1, 2, 3, 4 or 5;

each R₁ is independently selected from the group consisting of a side chain of a natural amino acid and a side chain of an unnatural amino acid, wherein the natural amino acid and the unnatural amino acid is either in the D or L form;

R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I and ¹³¹I;

W is selected from the group consisting of

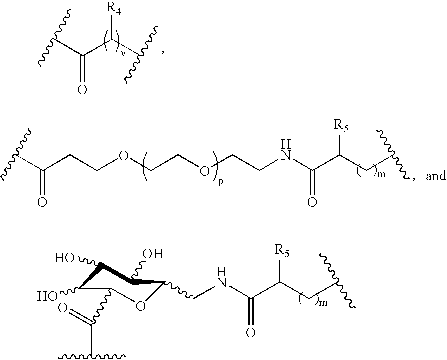

where p is 0 to 15; v is 0, 1, 2, or 3; m is 0, 1 or 2; each R₄ and R₅ is independently selected from the group consisting of —H, and optionally substituted C₁-C₆ alkyl; wherein the configuration of the chiral center that carries the R₅ substituent may be R or S or mixtures thereof.

A still further aspect of the present application is a method for imaging of blood vessel growth in solid tumors based on expression of integrin αᵥβ₃ within the body of a patient, the method comprising: (a) administering to the patient radiolabeled cyclic peptidomimetic; (b) employing a nuclear imaging technique selected from the group consisting of positron emission tomography (PET) and single photon emission computed tomography (SPECT) for imaging a distribution of the radiolabeled cyclic peptidomimetic within the body or within a portion thereof; and c) correlating the distribution of the radiolabeled cyclic peptidomimetic to the growth of blood vessels in solid tumors, wherein the radiolabeled cyclic peptidomimetic is selected from the group consisting of:

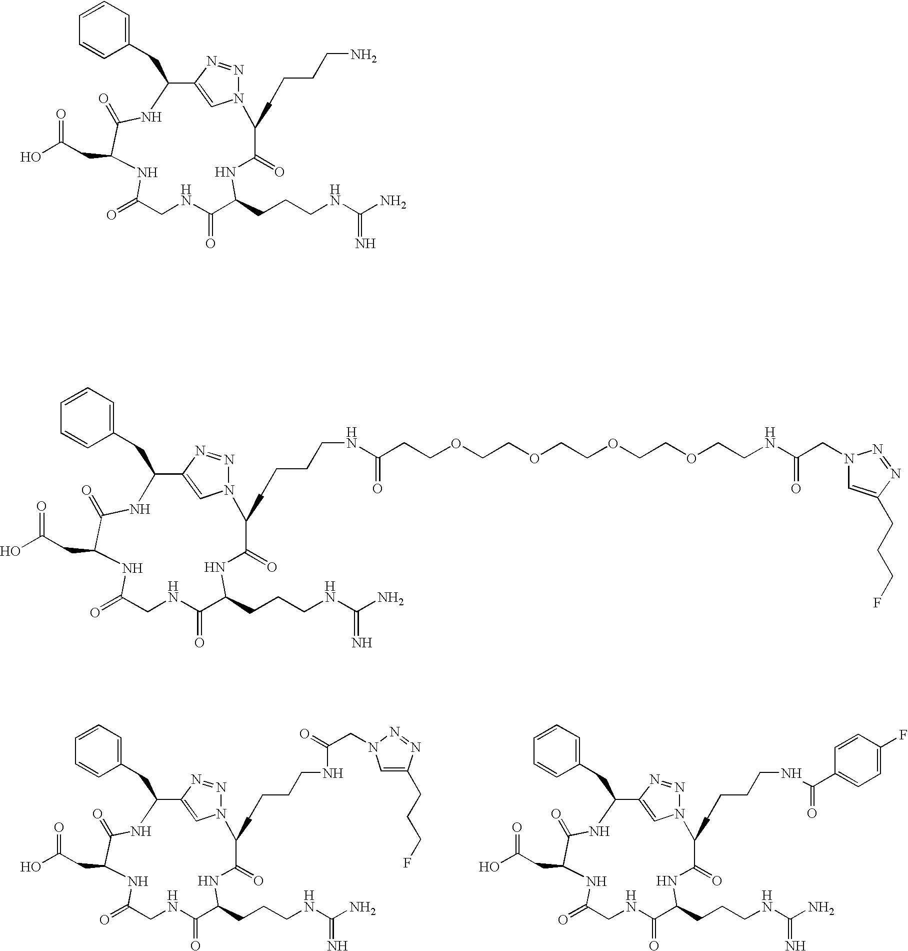

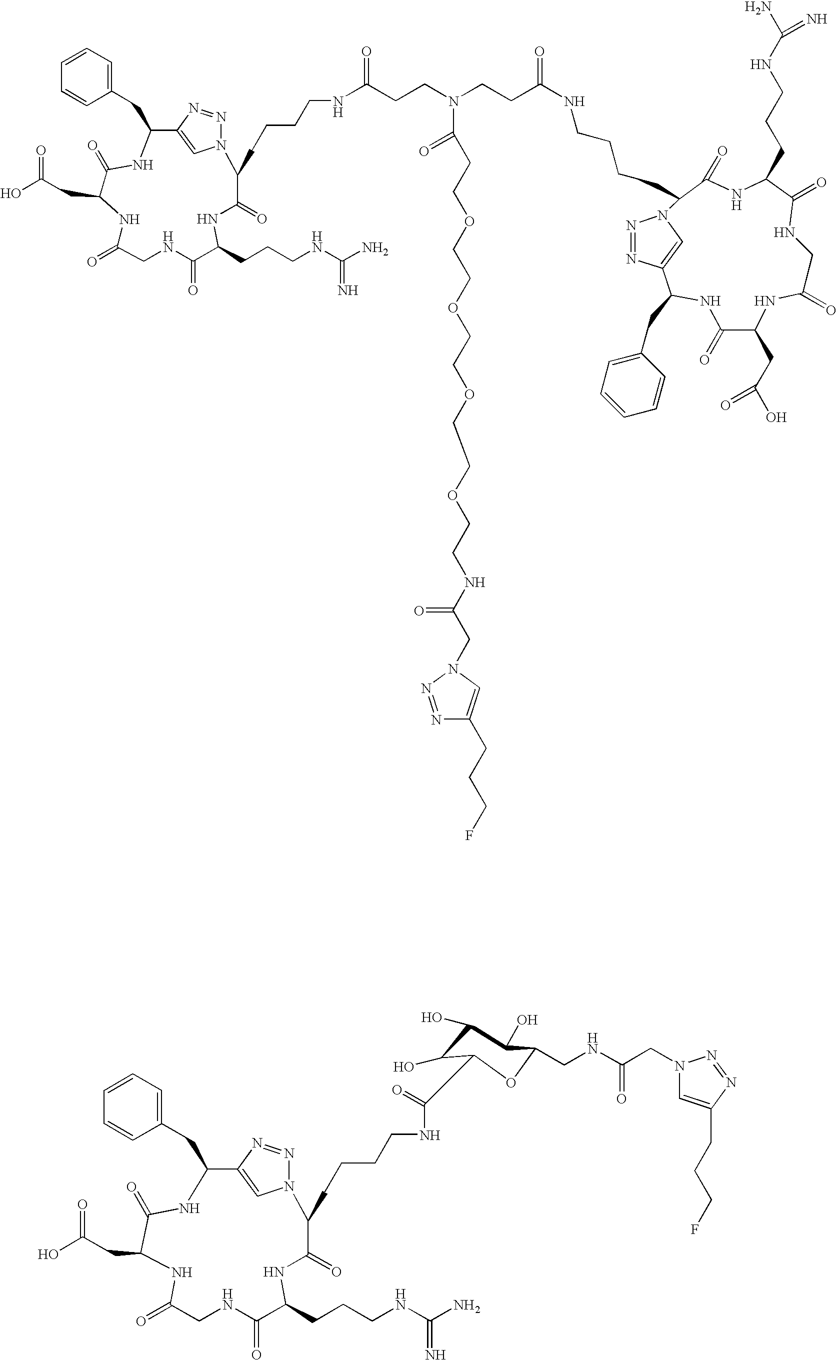

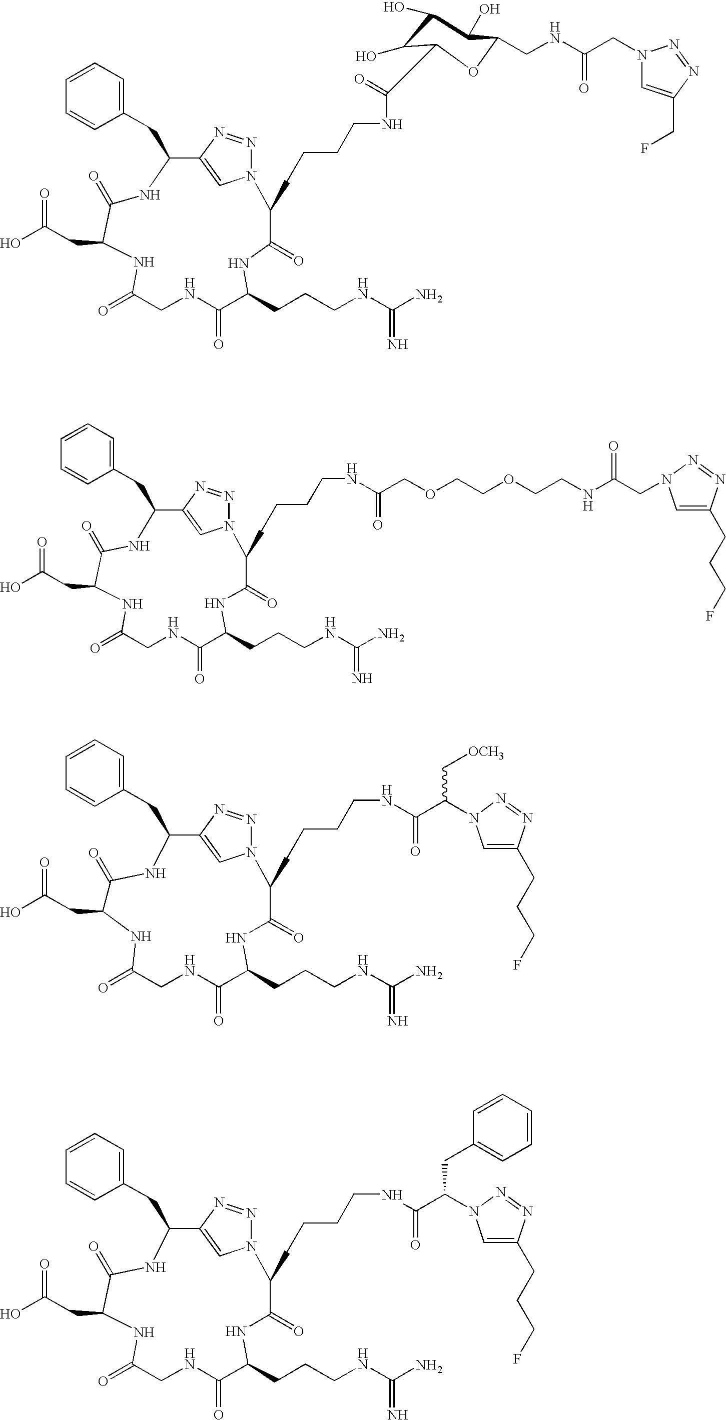

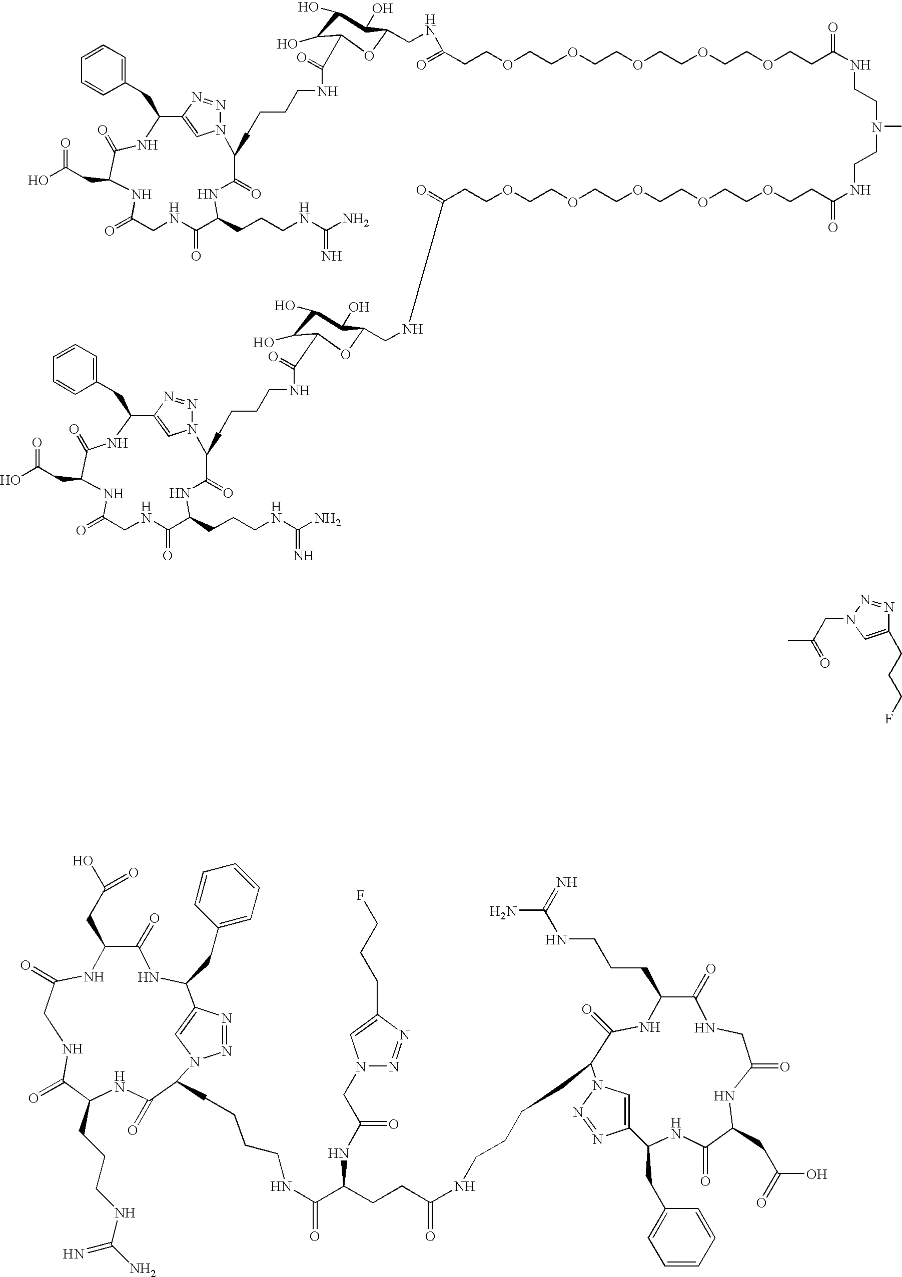

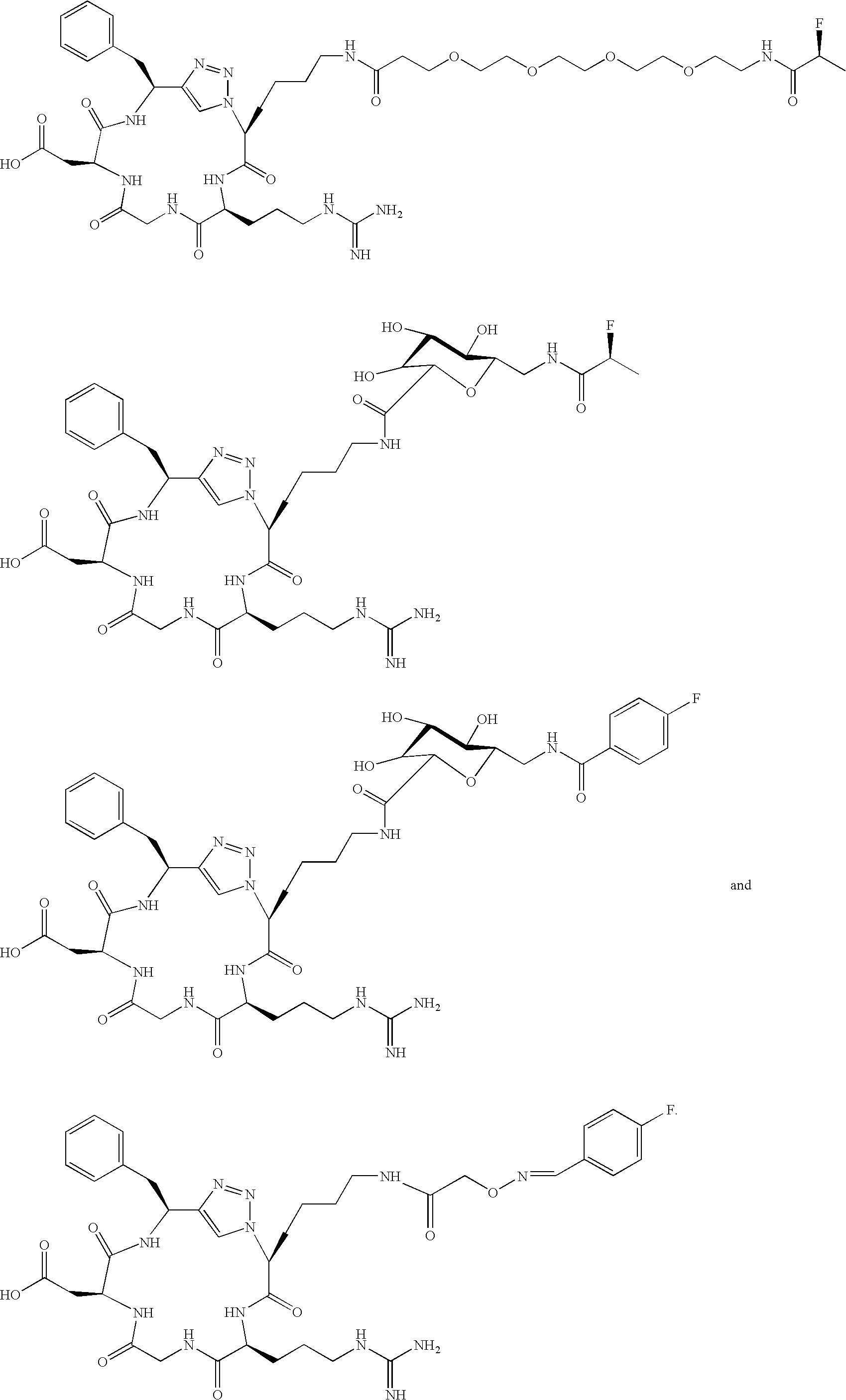

One aspect of the present application is a cyclic peptidomimetic having the structure

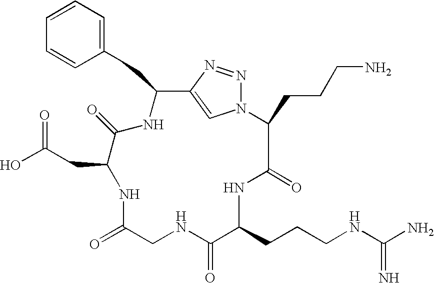

Another aspect of the present application is a cyclic peptidomimetic having the structure

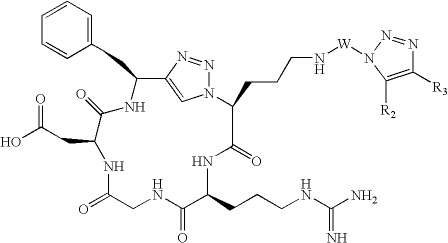

wherein R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, alkylene-aryl, carbocycle and heterocycle groups are optionally substituted; wherein R₃ and R₄ are not both H; and either R₃ or R₄, or both R₃ and R₄ comprise a radionuclide selected from the group consisting of positron emitters; W is a linker comprising a hydrophilic moiety selected from the group consisting of hydroxyl, carbonyl, sulfonamide, sulfonate, phosphate, a PEG moiety, sugar mimetic, and a sugar moiety.

Applicants have found that the cyclic peptidomimetics containing a 1,2,3-triazole, such as prepared via click chemistry can be dimerized. Such compounds demonstrate high binding affinity to integrin receptors and good pharmacokinetic properties. Thus, yet another aspect of the present application is a cyclic peptidomimetic of formula VI:

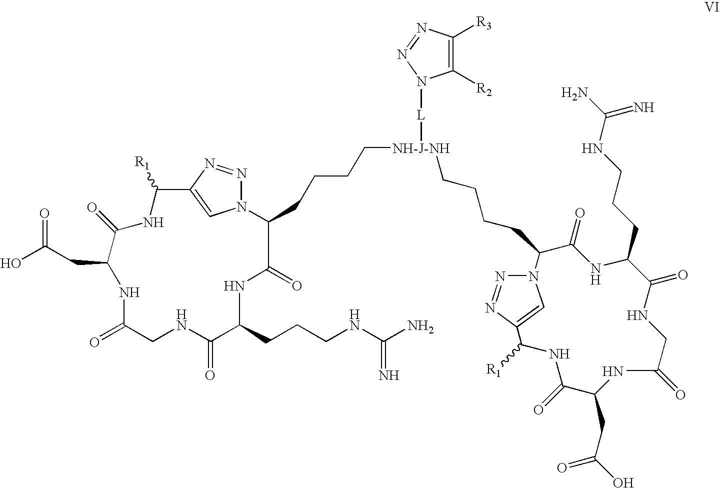

wherein each R₁ is independently selected from the group consisting of a side chain of a natural amino acid and a side chain of an unnatural amino acid, wherein the natural amino acid and the unnatural amino acid is either in the D or L form; R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl, carbocycle and heterocycle groups are each optionally substituted; wherein R₂ and R₃ are not both H; and either R₂ or R₃, or both R₂ and R₃ comprise a radionuclide selected from the group consisting of positron or gamma emitters; L is a linker comprising zero, one or more moieties selected from the group consisting of hydroxyl, carbonyl, sulfonamide, sulfonate, phosphate, polar amino acid moiety, PEG moiety, sugar mimetic, and a sugar moiety; J is a linker comprising a moiety selected from the group consisting of C₁-C₆ alkyl, —C₁-C₆ alkenyl, —C₁-C₆ alkynyl, aryl, aryl-(C₁-C₆ alkylene)-, 3- to 7-membered carbocycle, 3- to 7-membered heterocycle, and natural amino acids wherein the alkyl, alkenyl, alkynyl, aryl, carbocycle, heterocycle groups are each optionally substituted. In one embodiment, the radionuclide is selected from the group consisting of ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, ¹³¹I, ⁹⁹Tc, ⁷⁵Br, ¹⁵³Gd, and ³²P; L is selected from the group consisting of

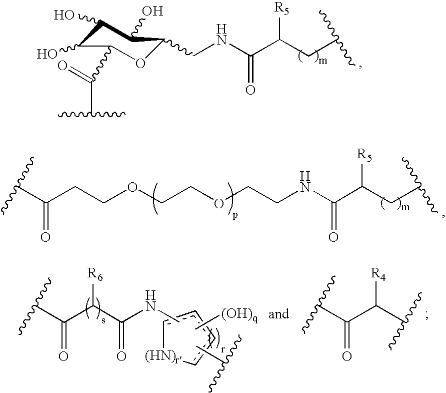

where R₄ is independently —H, —C₁-C₆ alkyl, C₁-C₆ alkenyl, C₁-C₆ alkynyl, C₁-C₆ alkyloxy, aryl, aryl-(C₁-C₆ alkylene)-, C₃-C₇ carbocycle, 3- to 7-membered heterocycle, hydroxy-C₁-C₆-alkyl, C₁-C₆-alkoxy-C₁-C₆-alkyl, and a PEG moiety, R₅ is selected from the group consisting of —H, C₁-C₆ alkyl, C₂-C₆ alkenyl, C₂-C₆ alkynyl, C₁-C₆ alkyloxy aryl, aryl-(C₁-C₆ alkylene)-, 3- to 7-membered carbocycle, 3- to 7-membered heterocycle, hydroxy-C₁-C₆-alkyl, and C₁-C₆-alkoxy-C₁-C₆-alkyl, wherein the alkyl, alkenyl, alkynyl, alkyloxy, aryl, carbocycle and heterocycle groups are each optionally substituted; each R₆ is independently selected from the group consisting of —H, —OH, C₁-C₆ alkyl, C₂-C₆ alkenyl, C₂-C₆ alkynyl, C₁-C₆ alkyloxy, aryl-(C₁-C₆ alkylene)-, hydroxy-C₁-C₆-alkyl, and C₁-C₆-alkoxy-C₁-C₆-alkyl, wherein the alkyl, alkenyl, alkynyl, alkyloxy, and aryl-alkylene groups are each optionally substituted; wherein the configuration of any of the chiral centers may optionally be R or S; q is 1, 2, 3 or 4; r is 1, 2 or 3; r′ is 0 or 1; s is 1, 2, 3 or 4; v is 0, 1, 2, 3, or 4; m is 0, 1, 2, 3, or 4; and p is an integer between 0 and 15; wherein either R₂ or R₃, or both R₂ and R₃ comprise a radionuclide selected from the group consisting of ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, ¹³¹I, ⁹⁹Tc, ⁷⁵Br, ¹⁵³Gd and ³²P. In one embodiment, the radionuclide is selected from the group consisting of ¹¹C, ¹³N, ¹⁵O, ¹⁸F, ¹²⁴I, ¹²⁵I, ¹³¹I, and ⁷⁵Br. In one variation, J is

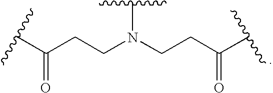

In another variation, J is

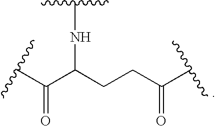

In one aspect, the peptidomimetic is of formula VII:

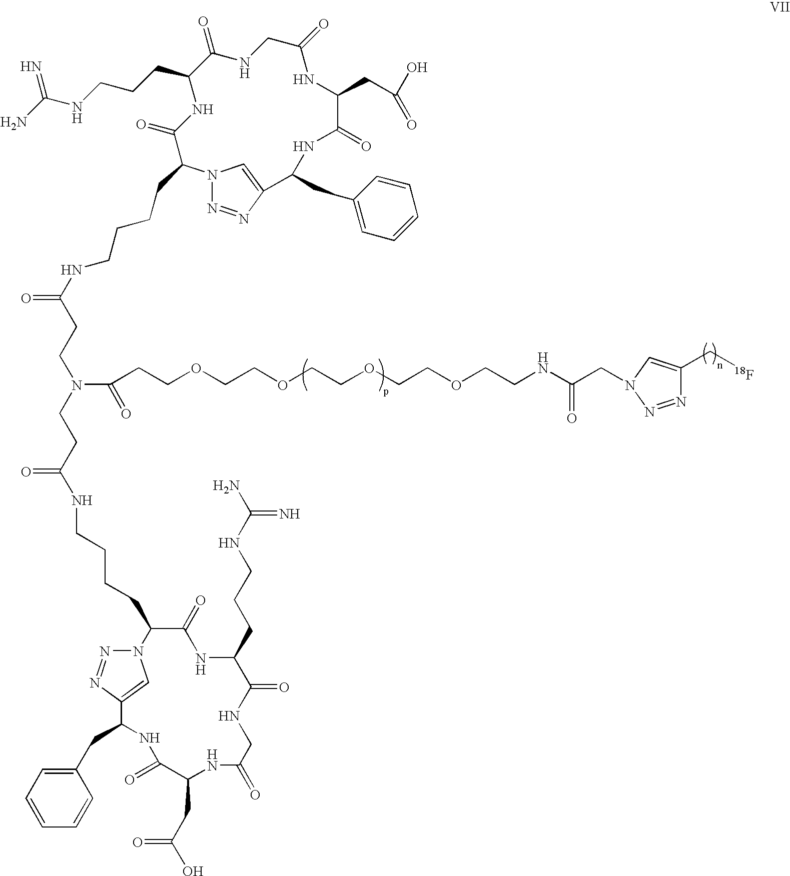

wherein p is 0, 1, 2, 3, 4, or 5 and n is 1, 2, 3, 4, or 5.

One aspect of the present application is a pharmaceutical composition comprising any of the above disclosed compounds and a pharmaceutically acceptable carrier. Another aspect of the present application the compounds disclosed herein can be used as tracers in Positron Emission Tomography (PET) or Single Photon Emission Computed Tomography (SPECT).

One aspect of the present application is a method of monitoring the level of integrin receptor within a body of a patient, the method comprising: (a) administering to the patient any of the above cited radiolabeled cyclic peptidomimetics, and (b) employing a nuclear imaging technique selected from the group consisting of positron emission tomography (PET) and single photon emission computed tomography (SPECT) for monitoring a distribution of the cyclic peptidomimetic within the body or within a portion thereof. In one embodiment, the integrin receptor is αᵥβ₃.

Another aspect of the present application is a method of visualizing integrin αᵥβ₃ expression within a body of a patient, the method comprising: (a) administering to the patient any of the above cited radiolabeled cyclic peptidomimetics; and (b) employing a nuclear imaging technique selected from the group consisting of positron emission tomography (PET) and single photon emission computed tomography (SPECT) for visualizing a distribution of the cyclic peptidomimetic within the body or within a portion thereof. In one embodiment, the integrin receptor is αᵥβ₃.

Another aspect of the present application is a method for imaging of blood vessel growth in solid tumors based on expression of integrin within the body of a patient, the method comprising: (a) administering to the patient any of the above cited the radiolabeled cyclic peptidomimetics; (b) employing a nuclear imaging technique selected from the group consisting of positron emission tomography (PET) and single photon emission computed tomography (SPECT) for imaging a distribution of the cyclic peptidomimetic within the body or within a portion thereof; and c) correlating the distribution of the cyclic peptidomimetic to the growth of blood vessels in solid tumors. In one embodiment, the integrin receptor is αᵥβ₃.

The integrin αᵥβ₃ plays an important role in regulating tumor growth and angiogenesis. The non-invasive visualization and quantification of αᵥβ₃ integrin levels in patients enables a variety of applications. One such application is determination of αᵥβ₃ levels before therapy with αᵥβ₃ antagonists. Patients with low or no αᵥβ₃ expression might not benefit from αᵥβ₃ antagonist therapy and could then receive alternate treatment. Patients with αᵥβ₃ positive lesions could have their treatment optimized, based on the use of the compounds of the present application to evaluate inhibition of the αᵥβ₃ integrin.

Pharmaceutical compositions of the compounds of this application, or derivatives thereof, may be formulated as solutions or lyophilized powders for parenteral administration. Powders may be reconstituted by addition of a suitable diluent or other pharmaceutically acceptable carrier prior to use. The liquid formulation is generally a buffered, isotonic, aqueous solution. Examples of suitable diluents are normal isotonic saline solution, 5% dextrose in water or buffered sodium or ammonium acetate solution. Such formulations are especially suitable for parenteral administration but may also be used for oral administration. Excipients, such as polyvinylpyrrolidinone, gelatin, hydroxycellulose, acacia, polyethylene glycol, mannitol, sodium chloride, or sodium citrate, may also be added. Alternatively, these compounds may be encapsulated, tableted, or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols, or water. Solid carriers include starch, lactose, calcium sulfate, dihydrate, terra alba, magnesium stearate or stearic acid, talc, pectin, acacia, agar, or gelatin. The carrier may also include a sustained release material such as glyceryl monostearate or glyceryl distearate, alone or with a wax. The pharmaceutical preparations are made following the conventional techniques of pharmacy involving milling, mixing, granulation, and compressing, when necessary, for tablet forms; or milling, mixing, and filling for hard gelatin capsule forms. When a liquid carrier is used, the preparation may be in the form of a syrup, elixir, emulsion, or an aqueous or non-aqueous suspension. Such a liquid formulation may be administered directly p.o. or filled into a soft gelatin capsule. Suitable formulations for each of these methods of administration may be found in, for example, REMINGTON: THE SCIENCE AND PRACTICE OF PHARMACY, A. Gennaro, ed., 20th edition, Lippincott, Williams & Wilkins, Philadelphia, Pa.

The pharmaceutical compositions of the application may be in the form of a sterile injectable preparation. Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents.

### EXAMPLES

An exemplary reaction scheme for forming a library of cyclic peptidomimetics such as compounds of formula IIIA, using solid phase synthesis techniques is shown in Scheme I.

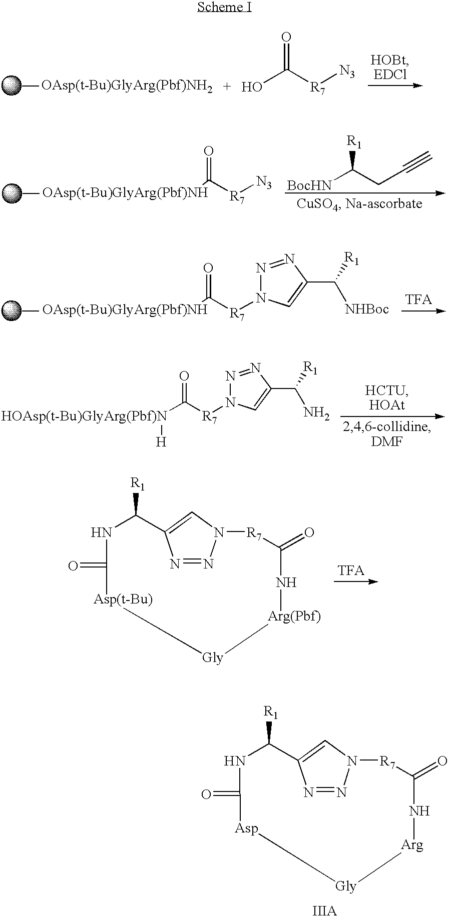

An exemplary reaction scheme for forming a cyclic peptidomimetics using solution-phase synthesis techniques is shown in Scheme II.

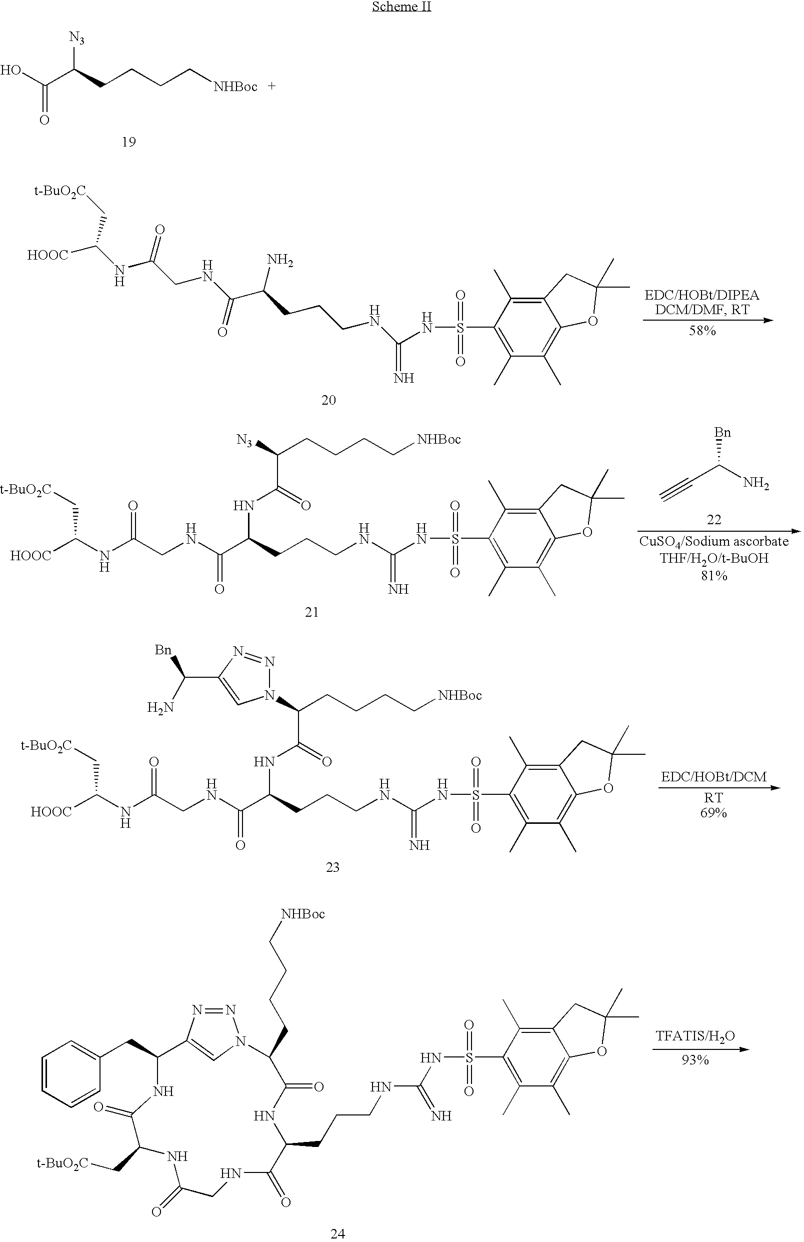

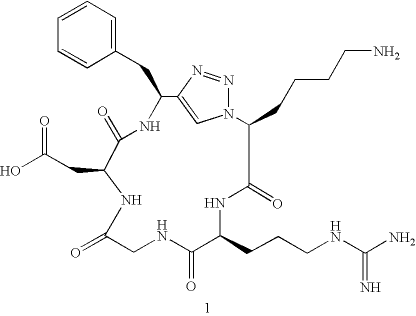

### Synthesis of Compound 1

Synthesis of Compound 21: (S)-2-Azido-6-(tert-butoxycarbonylamino)hexanoic acid 19 (3.12 g, 11.46 mmol) was dissolved in dichloromethane (CH₂Cl₂) (60 mL) and treated with 1-hydroxybenzotriazole (HOBt) (1.55 g, 11.46 mmol) and N-(3-Dimethylaminopropyl)-N′-ethylcarbodiimide hydrochloride (EDC) (2.21 gm, 11.46 mmol) at room temperature. After stirring for 2 hr, a solution of (S)-2-(2-((S)-2-amino-5-(3-(2,2,4,6,7-pentamethyl-2,3-dihydrobenzofuran-5-ylsulfonyl)guanidino)-pentanamido)acetamido)-4-tert-butoxy-4-oxobutanoic acid 20 (5 g, 7.64 mmol) in N,N′-dimethylformamide (DMF) (15 mL) and N,N′-diisopropylethylamine (DIPEA) (2.66 mL, 15.28 mmol) were added to the reaction mixture and stirred for 12 hr. LC/MS shows all the starting material was consumed. Solvent removed under high vacuum, and residue was dissolved in water (100 mL) and extracted three times with ethyl acetate (100 mL), washed with saturated brine and dried over MgSO₄. The solvent removed in vacuo, and the compound 21 (4.0 g, 58%) was isolated by chromatography on silica gel (MeOH/EtOAc, ⅕) as white solid. MS (m/z) (ESI): 909.7 [M+H]⁺.

Synthesis of Compound 23: A solution of compound 21 (4.15 g, 4.65 mmol) in t-BuOH:THF:H₂O (30 mL, 1:1:1) was treated with CuSO₄.5H₂O (0.06 g, 0.228 mmol), sodium ascorbate (0.09 g, 0.457 mmol) and (S)-1-phenylbut-3-yn-2-amine 22 (0.7 g, 4.79 mmol) at room temperature. After stirring the reaction mixture for 1 hr, solvents were removed under vacuum and the compound 23 (3.91 g, 81%) was isolated by chromatography on silica gel (MeOH/EtOAc, ¼) as white solid. MS (m/z) (ESI): 1054.6 [M+H]⁺.

Synthesis of Compound 24: A solution of compound 23 (3.91 g, 3.71 mmol) in CH₂Cl₂ (1173 mL) was treated with HOBt (0.55 g, 4.08 mmol) and EDC (0.78 g, 4.08 mmol) at room temperature. After stirring the reaction mixture for 12 hr, solvent removed under vacuum and the compound 25 (2.66 g, 69%) was isolated by chromatography on silica gel (MeOH/EtOAc, ⅕) as white solid. MS (m/z) (ESI): 1036.8 [M+H]⁺.

Synthesis of Compound 1: Compound 24 (2.66 g, 2.57 mmol) was treated with TFA:TIS:H₂O (100 mL, 95:2.5:2.5) at room temperature for 2.5 hr. Solvent removed under high vacuum, and the residue was washed 5 times with cold CH₃CN (30 mL), and dissolved in 50 mL of water. The aqueous layer was washed 3 times with cold EtOAc (30 mL), after removing the water compound 1 (1.5 g, 93%) was isolated in about 90% purity as white solid. ¹H NMR (MeOH d₄, 400 MHz): δ((ppm) 8.79 (d, J=6 Hz, 1H), 8.69 (m, 1H), 7.87 (s, 1H), 7.58 (d, J=8.8 Hz, 1H), 7.31-7.24 (m, 4H), 7.20-7.17 (m, 1H), 6.76 (d, J=7.2 Hz, 1H), 5.43 (app. q, J=7.6 Hz, 1H), 5.12 (dd, J=9.2, 5.2 Hz, 1H), 4.49-4.45 (m, 1H), 4.31-4.27 (m, 1H), 3.89 (dd, J=14.8, 10.8 Hz, 1H), 3.61 (dd, J=13.2, 6.4 Hz, 1H), 3.54-3.50 (m, 1H), 3.25-3.11 (m, 4H), 2.93 (app. t, J=7.2 Hz, 1H), 2.67-2.47 (m, 3H), 1.75 (m, 3H), 1.65-1.55 (m, 4H). ¹³C NMR (MeOH d₄, 400 MHz): δ((ppm) 172.8, 172.3, 171.1, 170.1, 157.5, 148.4, 137.9, 129.4, 128.1, 126.4, 125.2, 65.8, 52.8, 51.7, 48.7, 43.2, 40.6, 39.2, 39.0, 35.1, 29.2, 29.1, 26.7, 25.2, 23.1. MS (m/z) (ESI): 628.3 [M+H]⁺, 650.3 [M+Na]⁺.

Consistent with the synthetic schemes presented herein, a series of cyclic peptidomimetics derivatives was synthesized. See e.g. Table 1.

TABLE 1
Derivatives of cyclic peptidomimetics

| | | | Radiolabeling |
|---|---|---|---|
| Compound | Chemical Structure | MW | Method |
| 1 | 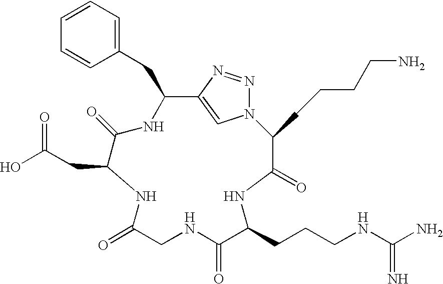 | 627.32 | — |
| 2 | 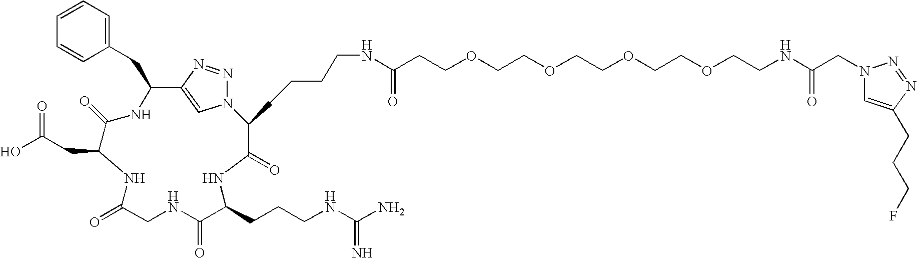 | 1043.53 | ClickChemistry |
| 3 | 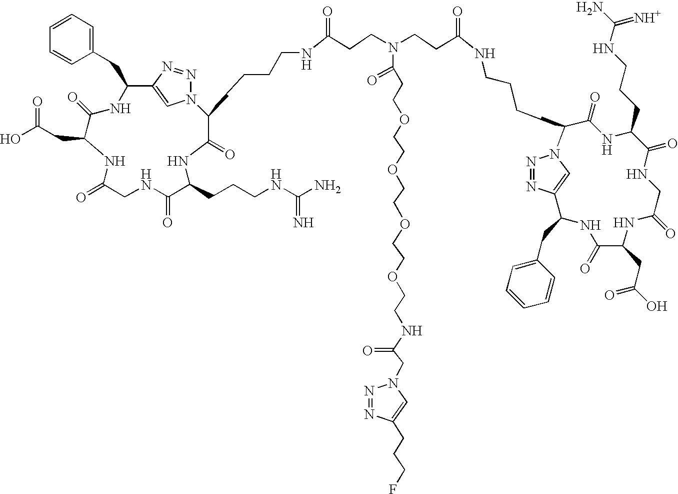 | 1795.90 | ClickChemistry |
| 4 | 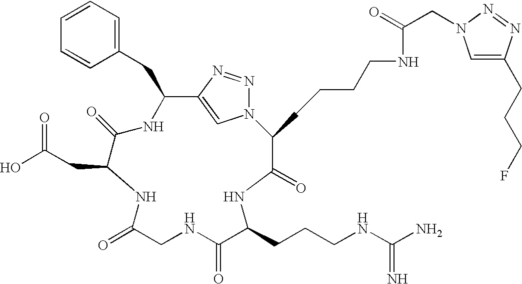 | 796.39 | ClickChemistry |
| 5 | 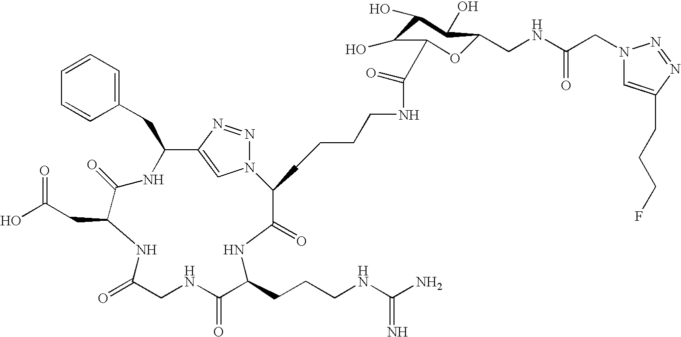 | 985.45 | ClickChemistry |
| 6 | 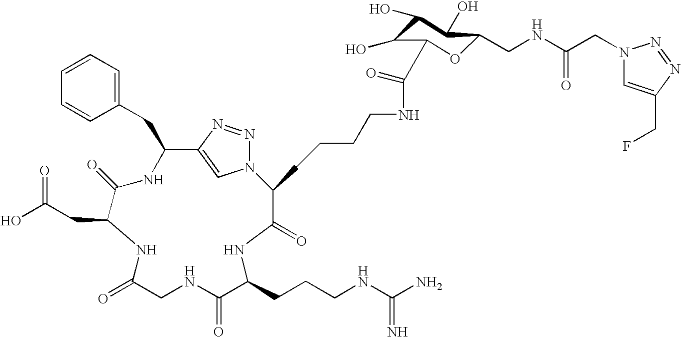 | 957.42 | ClickChemistry |
| 7 | 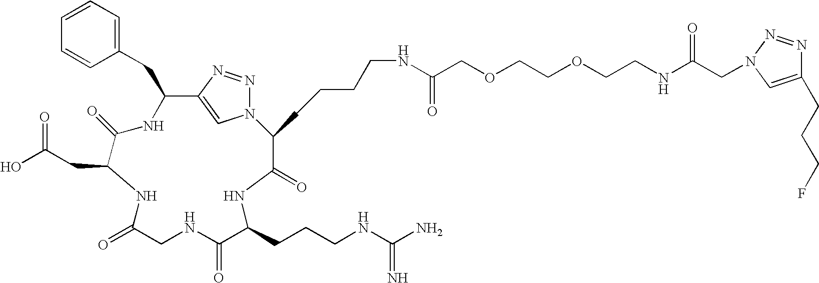 | 941.46 | ClickChemistry |
| 8 | 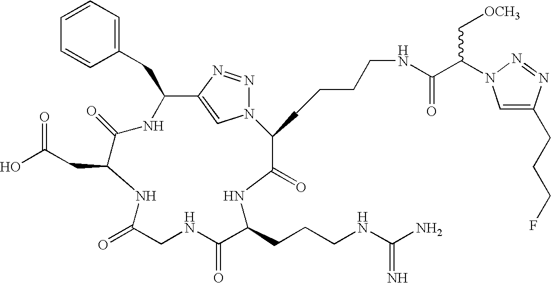 | 840.42 | ClickChemistry |
| 9 | 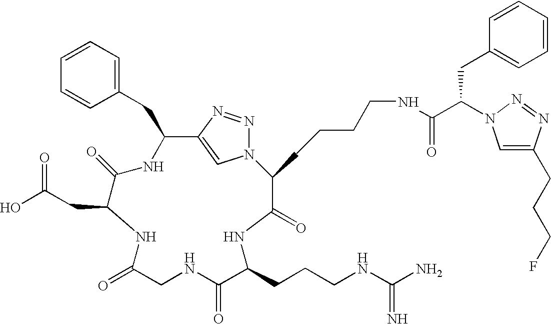 | 886.44 | ClickChemistry |
| 10 | 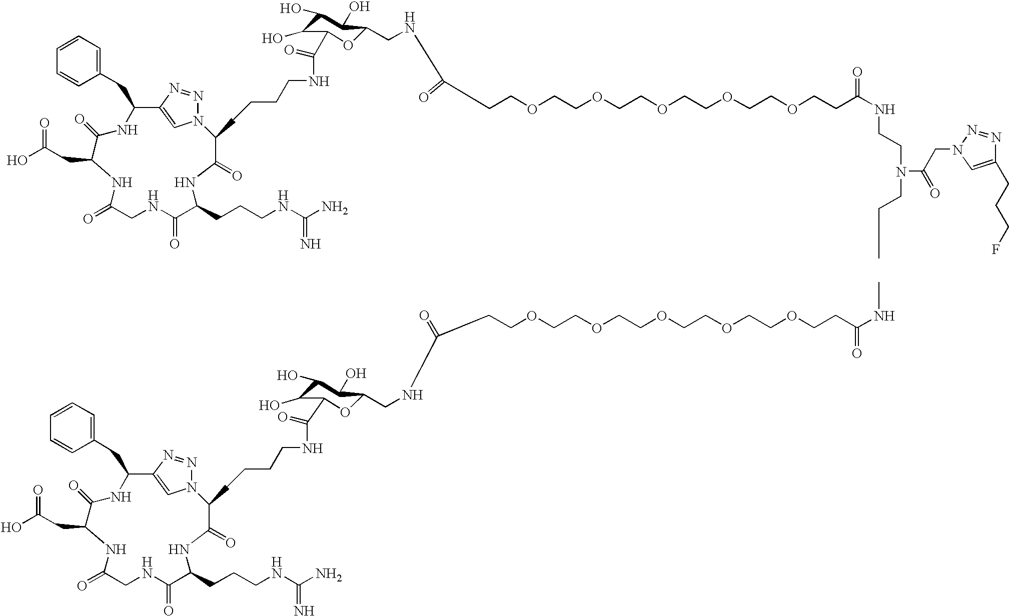 | 2509.22 | ClickChemistry |
| 11 | 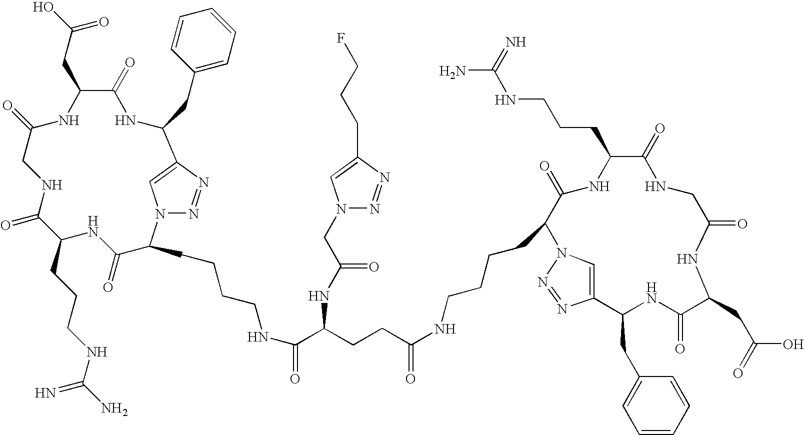 | 1534.75 | ClickChemistry |
| 12 | 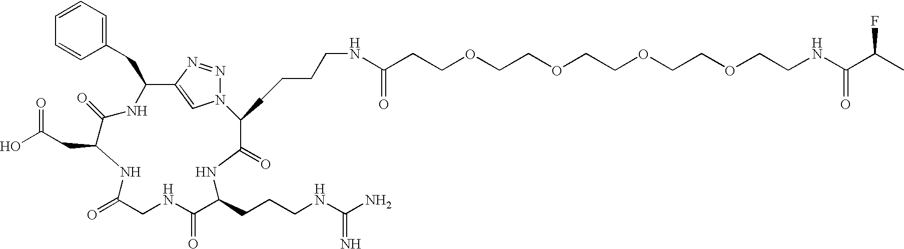 | 948.48 | Amidecoupling |
| 13 | 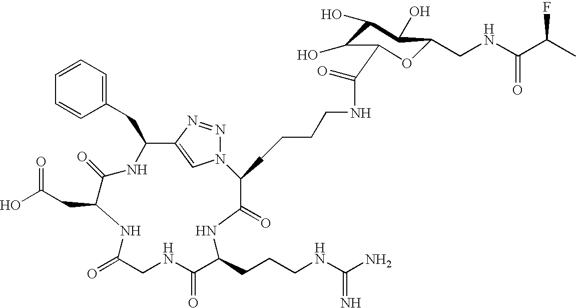 | 890.40 | Amidecoupling |
| 14 | 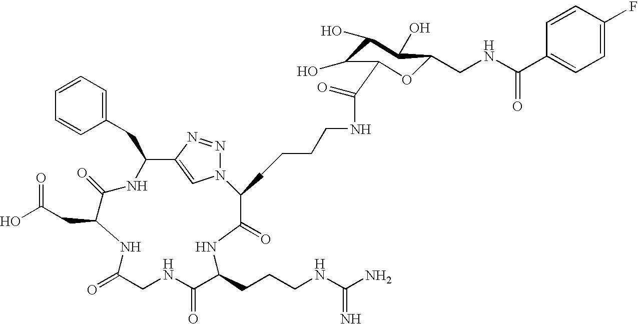 | 938.40 | Amidecoupling |
| 15 | 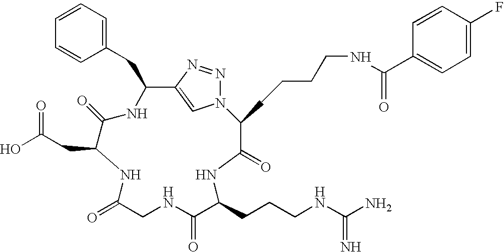 | 749.34 | Amidecoupling |
| 16 | 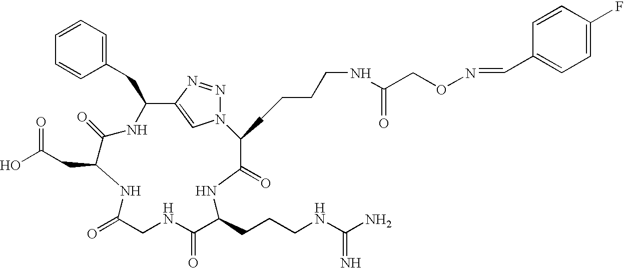 | 806.36 | Oximecoupling |

An exemplary preparation of one of the cyclic peptidomimetics derivatives of the present application, Compound 2, is shown in Scheme III.

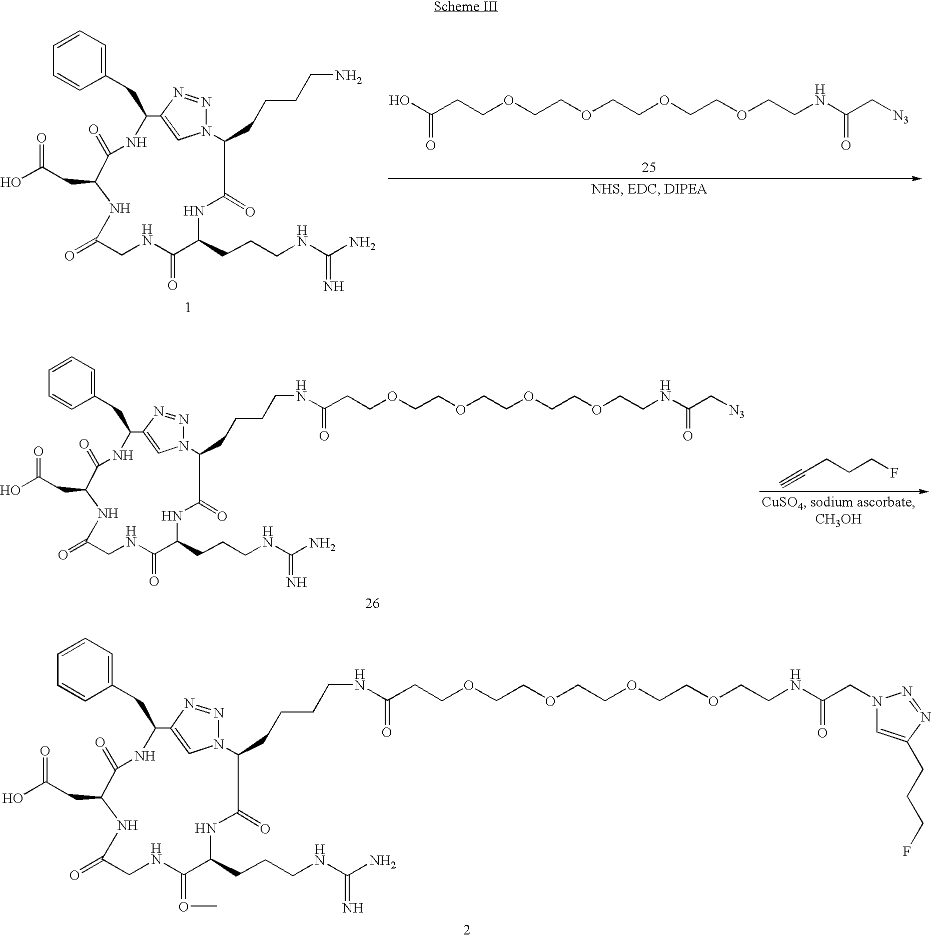

### Synthesis of Compound 2

Synthesis of Compound 26: To a solution of compound 1 (25 mg, 0.04 mmol) in DMF (2 mL), compound 26 (17.3 mg, 0.05 mmol) was added, followed by DIPEA (14 μL, 0.08 mmol). The reaction mixture was stirred for 12 hr at room temperature. LC/MS shows the starting material was consumed. Solvent was removed under high vacuum, and the residue was dissolved in water (5 mL). After filtration, the desired product was isolated by semi-preparative HPLC. The collected fractions were combined and lyophilized to afford compound 26 (13 mg, 34%) as a white fluffy powder. MS (m/z) (ESI): 958.7 [M+H]⁺.

Synthesis of Compound 2: To a small vial containing compound 26 (1.5 mg, 1.6 μmol), 5-fluoropent-1-yne (25 μL), CH₃OH (400 μL), and sodium ascorbate solution (25 μL, 0.5 M) was added copper sulfate solution (25 μL, 0.1 M). The reaction was stirred at room temperature for 2 hr. The reaction mixture was then concentrated to dryness and redissolved in water (3 mL). After filtration, the desired product was isolated by semi-preparative HPLC. The collected fractions were combined and lyophilized to afford compound 2 (1 mg, 63%) as a white fluffy powder. MS (m/z) (ESI): 1044.5 [M+H]⁺.

Another exemplary preparation of one of the cyclic peptidomimetics derivatives of the present application, Compound 3, is shown in Scheme IV. Scheme IV

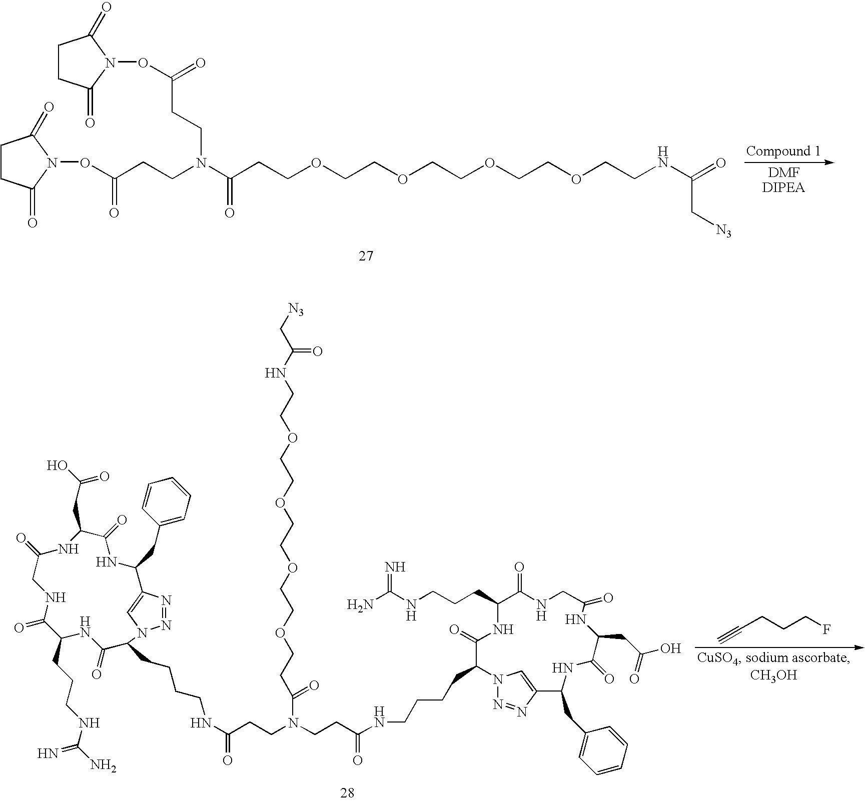

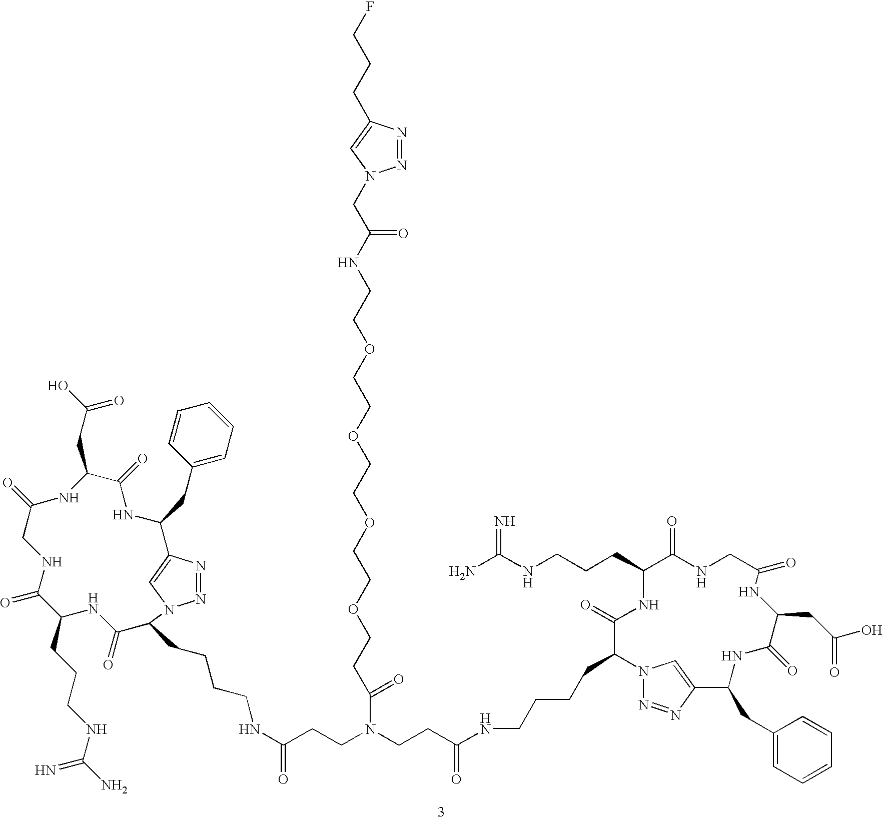

### Synthesis of Compound 3:

Synthesis of Compound 28: To a solution of compound 27 (22 mg, 0.032 mmol) in DMF (2 mL), compound 1 (50 mg, 0.08 mmol) was added, followed by DIPEA (17 μL, 0.096 mmol). The reaction mixture was stirred for 12 hr at room temperature. LC/MS shows the starting material was consumed. After solvent was removed under high vacuum, the residue was dissolved in water (3 mL) and actonitrile (3 mL). After filtration, the desired product was isolated by semi-preparative HPLC. The collected fractions were combined and lyophilized to afford compound 28 (8 mg, 15%) as a white fluffy powder. MS (m/z) (ESI): 1710.1 [M+H]⁺.

Synthesis of Compound 3: To a small vial containing compound 28 (2 mg, 1.2 μmol), 5-fluoropent-1-yne (25 μL), CH₃OH (400 μL), and sodium ascorbate solution (25 μL, 0.5 M) was added copper sulfate solution (25 μL, 0.1 M). The reaction was stirred at room temperature for 2 hr. The reaction mixture was then concentrated to dryness and redissolved in water (3 mL). After filtration, the desired product was isolated by semi-preparative HPLC. The collected fractions were combined and lyophilized to afford compound 3 (1.1 mg, 52%) as a white fluffy powder. MS (m/z) (ESI): 898.9 [M/2+H]⁺.

Another exemplary preparation of one of the cyclic peptidomimetics derivatives of the present application, Compound 5, is shown in Scheme V.

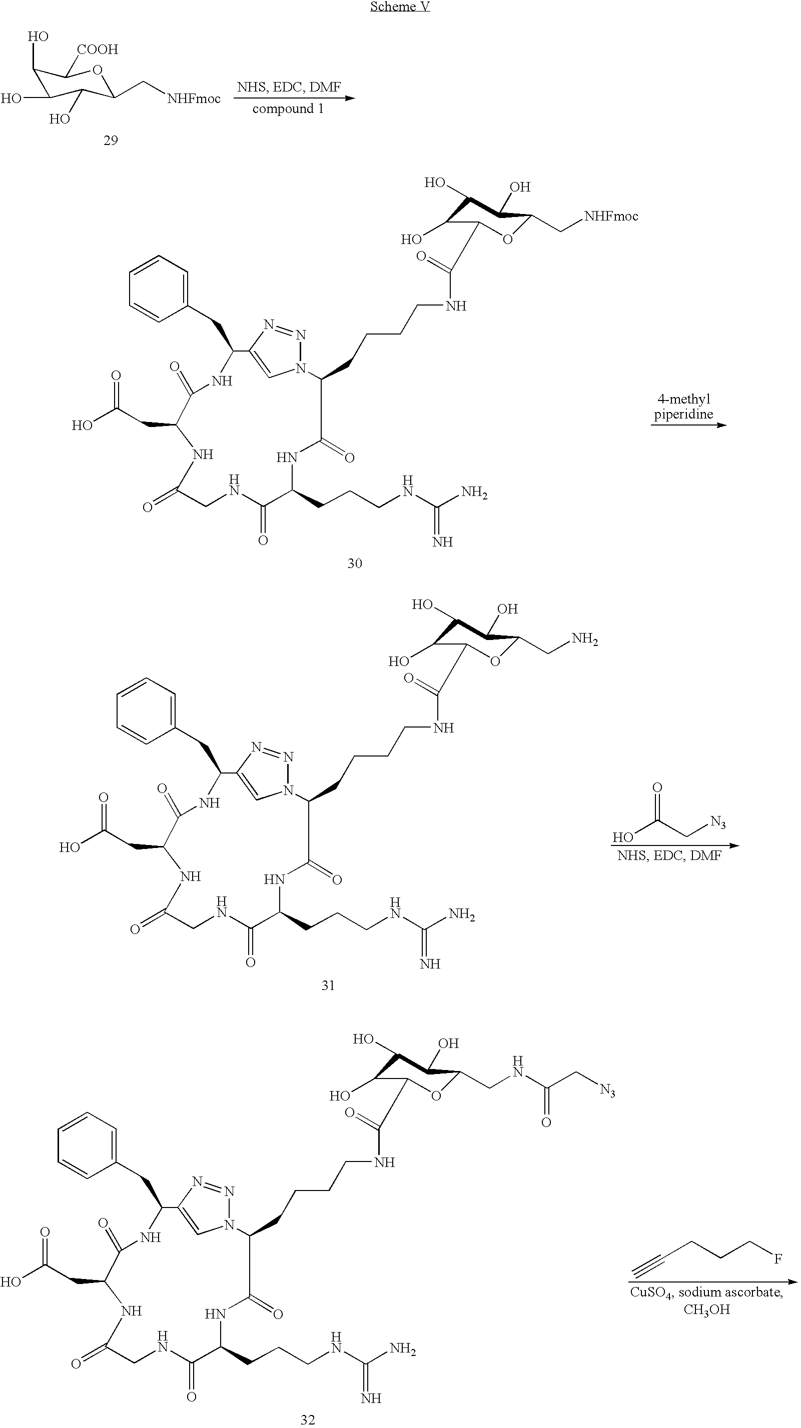

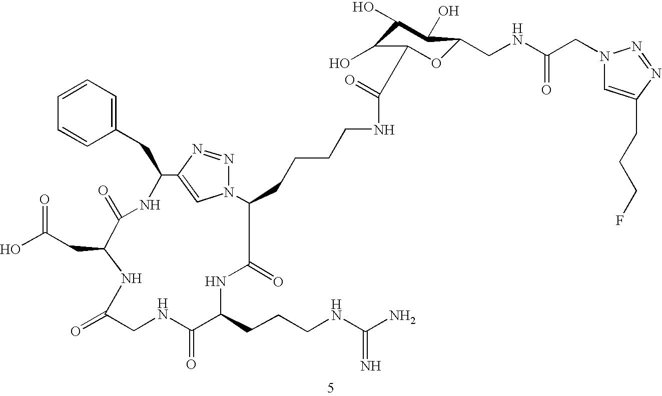

### Synthesis of Compound 5:

Synthesis of Compound 30: 6-((((9H-fluoren-9-yl)methoxy)carbonylamino)-methyl)-3,4,5-trihydroxytetrahydro-2H-pyran-2-carboxylic acid (29) [Ref. 7] (44.46 mg, 0.104 mmol) was dissolved in N,N′-dimethylformamide (DMF) (2 mL) and treated with N-hydroxysuccinimide (NHS) (12 mg, 0.104 mmol) and N-(3-Dimethylaminopropyl)-N′-ethylcarbodiimide hydrochloride (EDC) (19.9 mg, 0.104 mmol) at room temperature. After stirring for 1 hr, a solution of compound 1 (52 mg, 0.083 mmol) in DMF (1 mL) and N,N′-diisopropylethylamine (DIPEA) (20 μL, 0.115 mmol) were added to the reaction mixture and stirred for 6 hr. LCMS shows all the starting material was consumed. Solvent was removed under high vacuum, and residue was dissolved in water (10 mL) and methanol (2 mL). After filtration, the desired product was isolated by semi-preparative HPLC. The collected fractions were combined and lyophilized to afford compound 4 (28 mg, 33%) as a white fluffy powder. MS (m/z) (ESI): 1039.3 [M+H]⁺.

Synthesis of Compound 31: Compound 30 (28 mg, 0.027 mmol) was treated with 20% 4-methyl piperidine in DMF (5 ml) for 2 hr at room temperature. After removing the solvent under high vacuum, the residue was dissolved in water (5 mL). After filtration, the desired product was isolated by semi-preparative HPLC. The collected fractions were combined and lyophilized to afford compound 5 (20 mg, 90%) as a white fluffy powder. MS (m/z) (ESI): 817.5 [M+H]⁺, 839.5 [M+Na]⁺.

Synthesis of Compound 32: 2-Azidoacetic acid (100 mg, 0.046 mmol, 5% w/w in dichloromethane) was dissolved in DMF (1 mL) and treated with NHS (5.29 mg, 0.046 mmol) and EDC (8.81 mg, 0.046 mmol) at room temperature. After stirring for 1 hr, a solution of compound 31 (30 mg, 0.037 mmol) in DMF (1 mL) and DIPEA (16 μL, 0.092 mmol) were added to the reaction mixture and stirred for 3 hr. LCMS shows all the starting material was consumed. Solvent was removed under high vacuum, and residue was dissolved in water (3 mL). After filtration, the desired product was isolated by semi-preparative HPLC. The collected fractions were combined and lyophilized to afford compound 2 (14 mg, 43%) as a white fluffy powder. MS (m/z) (ESI): 900.2 [M+H]⁺, 922.0 [M+Na]⁺.

Synthesis of Compound 5: To a small vial containing compound 2 (4.0 mg, 4.45 μmol), 5-fluoropent-1-yne (25 μL), CH₃OH (400 μL), and sodium ascorbate solution (25 μL, 0.5 M) was added copper sulfate solution (25 μL, 0.1 M). The reaction was stirred at room temperature for 2 hr. The reaction mixture was then concentrated to dryness and redissolved in water (3 mL). After filtration, the desired product was isolated by semi-preparative HPLC. The collected fractions were combined and lyophilized to afford compound 1 (3.0 mg, 70%) as a white fluffy powder. MS (m/z) (ESI): 986.3 [M+H]⁺.

Another exemplary preparation of one of the cyclic peptidomimetics derivatives of the present application, Compound 7, is shown in Scheme VI.

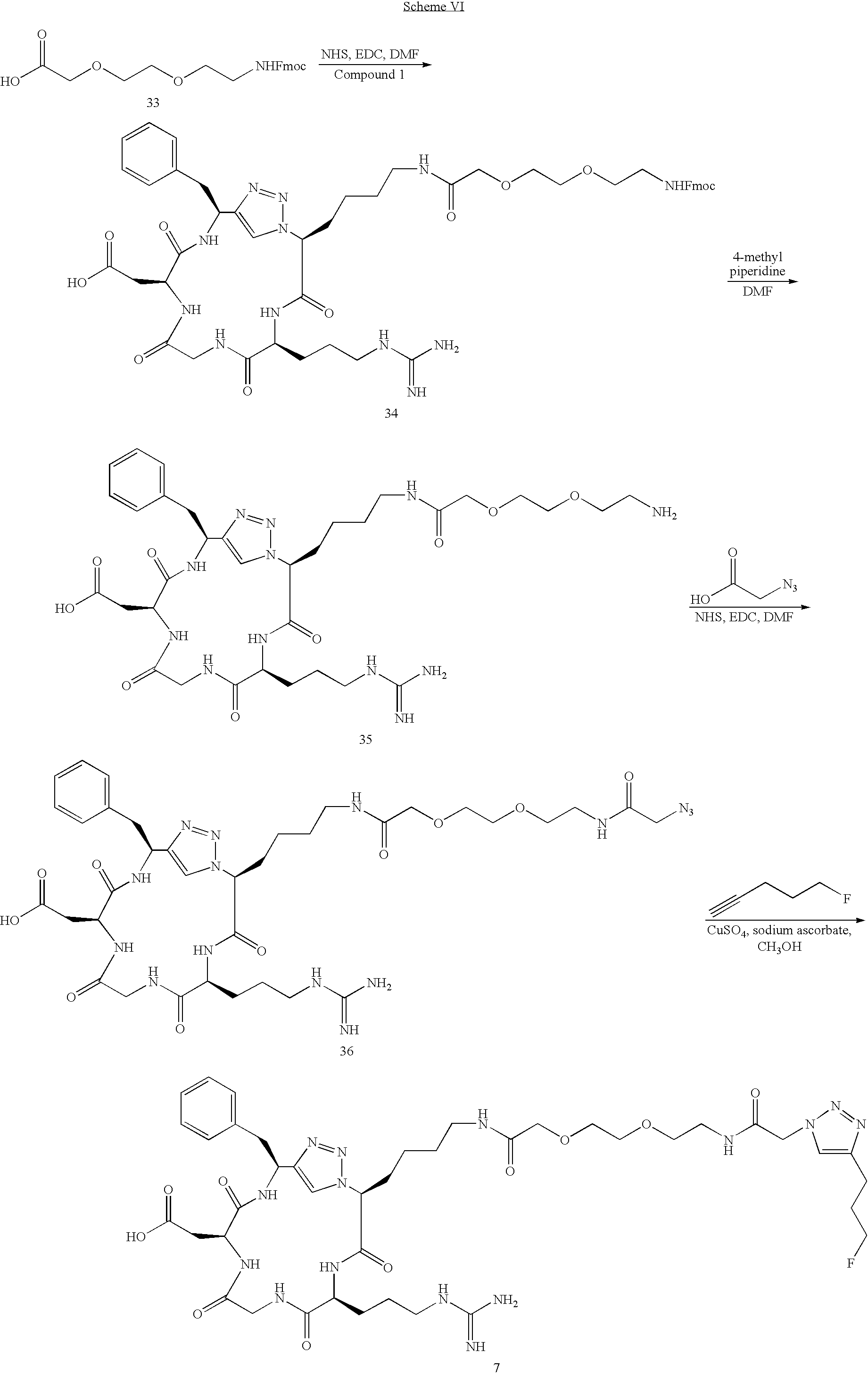

### Synthesis of Compound 7:

Synthesis of Compound 34: 1-(9H-fluoren-9-yl)-3-oxo-2,7,10-trioxa-4-azadodecan-12-oic acid 33 (23 mg, 0.06 mmol) was dissolved in N,N′-dimethylformamide (DMF) (2 mL) and treated with N-hydroxysuccinimide (NHS) (6.9 mg, 0.06 mmol) and N-(3-Dimethylaminopropyl)-N′-ethylcarbodiimide hydrochloride (EDC) (11.5 mg, 0.06 mmol) at room temperature. After stirring for 1 hr, a solution of compound 1 (30 mg, 0.048 mmol) in DMF (1 mL) and N,N′-diisopropylethylamine (DIPEA) (20 μL, 0.12 mmol) were added to the reaction mixture and stirred for 6 hr. LC/MS shows all the starting material was consumed. Solvent was removed under high vacuum, and residue was dissolved in water (10 mL) and methanol (2 mL). After filtration, the desired product was isolated by semi-preparative HPLC. The collected fractions were combined and lyophilized to afford compound 34 (17 mg, 35%) as a white fluffy powder. MS (m/z) (ESI): 995.5 [M+H]+.

Synthesis of Compound 35: Compound 34 (17 mg, 0.017 mmol) was treated with 20% 4-methyl piperidine in DMF (5 ml) for 2 hr at room temperature. After removing the solvent under high vacuum, the residue was dissolved in water (5 mL). After filtration, the desired product was isolated by semi-preparative HPLC. The collected fractions were combined and lyophilized to afford compound 35 (12 mg, 91%) as a white fluffy powder. MS (m/z) (ESI): 773.4 [M+H]+.

Synthesis of Compound 36: 2-Azidoacetic acid (39 mg, 0.019 mmol, 5% w/w in dichloromethane) was dissolved in DMF (1 mL) and treated with NHS (2.19 mg, 0.019 mmol) and EDC (3.64 mg, 0.019 mmol) at room temperature. After stirring for 1 hr, a solution of compound 35 (12 mg, 0.016 mmol) in DMF (1 mL) and DIPEA (15 μL, 0.086 mmol) were added to the reaction mixture and stirred for 3 hr. LCMS shows all the starting material was consumed. Solvent was removed under high vacuum, and residue was dissolved in water (3 mL). After filtration, the desired product was isolated by semi-preparative HPLC. The collected fractions were combined and lyophilized to afford compound 36 (6.1 mg, 45%) as a white fluffy powder. MS (m/z) (ESI): 856.4 [M+H]+.

Synthesis of Compound 7: To a small vial containing compound 36 (2 mg, 2.34 μmol), 5-fluoropent-1-yne (25 μL), CH3OH (400 μL), and sodium ascorbate solution (25 μL, 0.5 M) was added copper sulfate solution (25 μL, 0.1 M). The reaction was stirred at room temperature for 2 hr. The reaction mixture was then concentrated to dryness and redissolved in water (3 mL). After filtration, the desired product was isolated by semi-preparative HPLC. The collected fractions were combined and lyophilized to afford compound 7 (1.7 mg, 75%) as a white fluffy powder. MS (m/z) (ESI): 942.5 [M+H]+.

Another exemplary preparation of one of the cyclic peptidomimetics derivatives of the present application, Compound 8, is shown in Scheme VII.

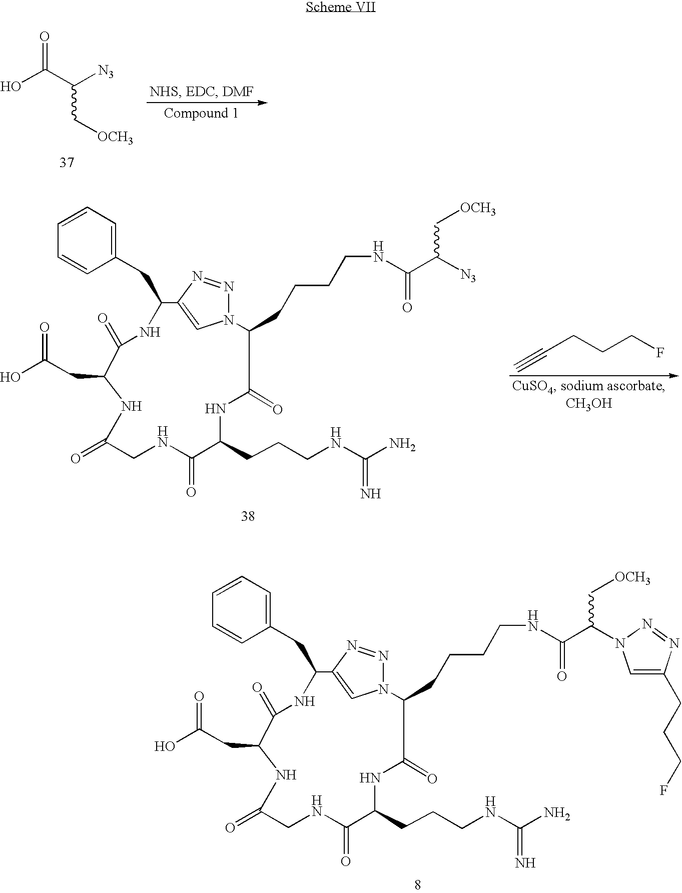

### Synthesis of Compound 8:

Synthesis of Compound 38: 2-azido-3-methoxypropanoic acid (5.78 mg, 0.04 mmol) was dissolved in DMF (1 mL) and treated with NHS (4.59 mg, 0.04 mmol) and EDC (7.64 mg, 0.04 mmol) at room temperature. After stirring for 1 hr, a solution of compound 1 (20 mg, 0.032 mmol) in DMF (1 mL) and DIPEA (15 μL, 0.08 mmol) were added to the reaction mixture and stirred for 3 hr. LCMS shows all the starting material was consumed. Solvent was removed under high vacuum, and residue was dissolved in water (3 mL). After filtration, the desired product was isolated by semi-preparative HPLC. The collected fractions were combined and lyophilized to afford compound 38 (11 mg, 46%) as a white fluffy powder. MS (m/z) (ESI): 755.4 [M+H]⁺, 777.4 [M+Na]⁺.

Synthesis of Compound 8: To a small vial containing compound 38 (2 mg, 2.65 μmol), 5-fluoropent-1-yne (25 μL), CH₃OH (400 μL), and sodium ascorbate solution (25 μL, 0.5 M) was added copper sulfate solution (25 μL, 0.1 M). The reaction was stirred at room temperature for 2 hr. The reaction mixture was then concentrated to dryness and redissolved in water (3 mL). After filtration, the desired product was isolated by semi-preparative HPLC. The collected fractions were combined and lyophilized to afford compound 8 (1.6 mg, 71%) as a white fluffy powder. MS (m/z) (ESI): 841.4 [M+H]⁺, 863.4 [M+Na]⁺.

Another exemplary preparation of one of the cyclic peptidomimetics derivatives of the present application, Compound 12, is shown in Scheme VIII.

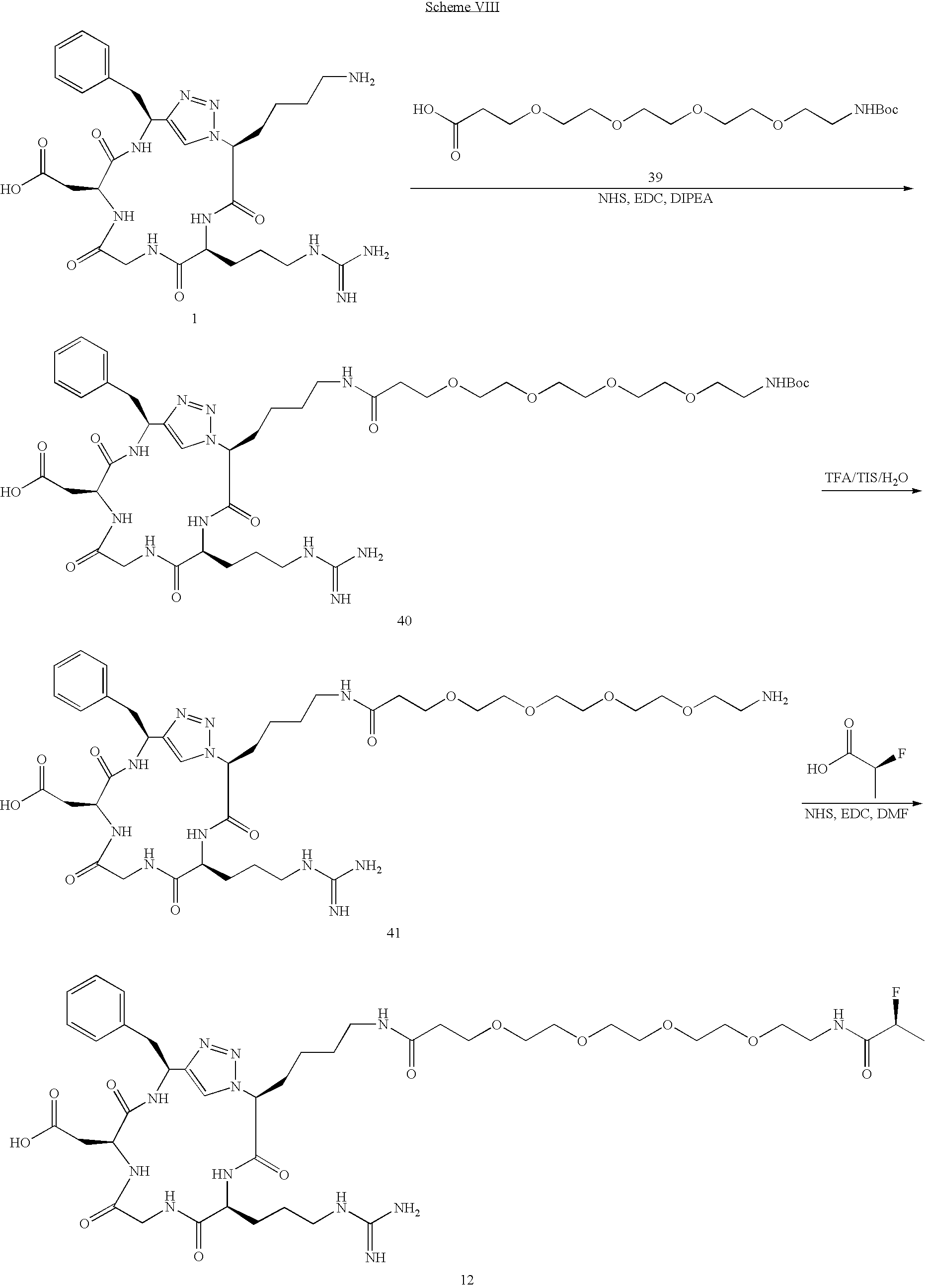

### Synthesis of Compound 12:

Synthesis of Compound 40: 2,2-Dimethyl-4-oxo-3,8,11,14,17-pentaoxa-5-azaicosan-20-oic acid 39 (35 mg, 0.096 mmol) was dissolved in N,N′-dimethylformamide (DMF) (2 mL) and treated with N-hydroxysuccinimide (NHS) (11 mg, 0.096 mmol) and N-(3-Dimethylaminopropyl)-N′-ethylcarbodiimide hydrochloride (EDC) (18.4 mg, 0.096 mmol) at room temperature. After stirring for 1 hr, a solution of compound 1 (30 mg, 0.048 mmol) in DMF (1 mL) and N,N′-diisopropylethylamine (DIPEA) (20 μL, 0.12 mmol) were added to the reaction mixture and stirred for 6 hr. LC/MS shows all the starting material was consumed. Solvent was removed under high vacuum, and residue was dissolved in water (10 mL) and methanol (2 mL). After filtration, the desired product was isolated by semi-preparative HPLC. The collected fractions were combined and lyophilized to afford compound 40 (21 mg, 46%) as a white fluffy powder. MS (m/z) (ESI): 975.3 [M+H]⁺.

Synthesis of Compound 41: Compound 40 (17 mg, 0.017 mmol) was treated with TFA:TIS:H₂O (100 mL, 95:2.5:2.5) at room temperature for 2 hr. After removing the solvent under high vacuum, the residue was dissolved in water (5 mL). After filtration, the desired product was isolated by semi-preparative HPLC. The collected fractions were combined and lyophilized to afford compound 41 (14 mg, 95%) as a white fluffy powder. MS (m/z) (ESI): 874.9 [M+H]⁺, 896.8 [M+Na]⁺.

Synthesis of Compound 12: 2-Fluoropropanoic acid (1.26 mg, 0.014 mmol) was dissolved in DMF (1 mL) and treated with NHS (1.58 mg, 0.014 mmol) and EDC (2.63 mg, 0.014 mmol) at room temperature. After stirring for 0.5 hr, a solution of compound 41 (3 mg, 3.43 μmol) in DMF (1 mL) and DIPEA (10 μL, 0.06 mmol) were added to the reaction mixture and stirred for 6 hr. LC/MS shows all the starting material was consumed. Solvent was removed under high vacuum, and residue was dissolved in water (3 mL). After filtration, the desired product was isolated by semi-preparative HPLC. The collected fractions were combined and lyophilized to afford compound 12 (1.8 mg, 56%) as a white fluffy powder. MS (n/z) (ESI): 848.8 [M+H]⁺, 870.8 [M+Na]⁺.

Another exemplary preparation of one of the cyclic peptidomimetics derivatives of the present application, Compound 13, is shown in Scheme IX.

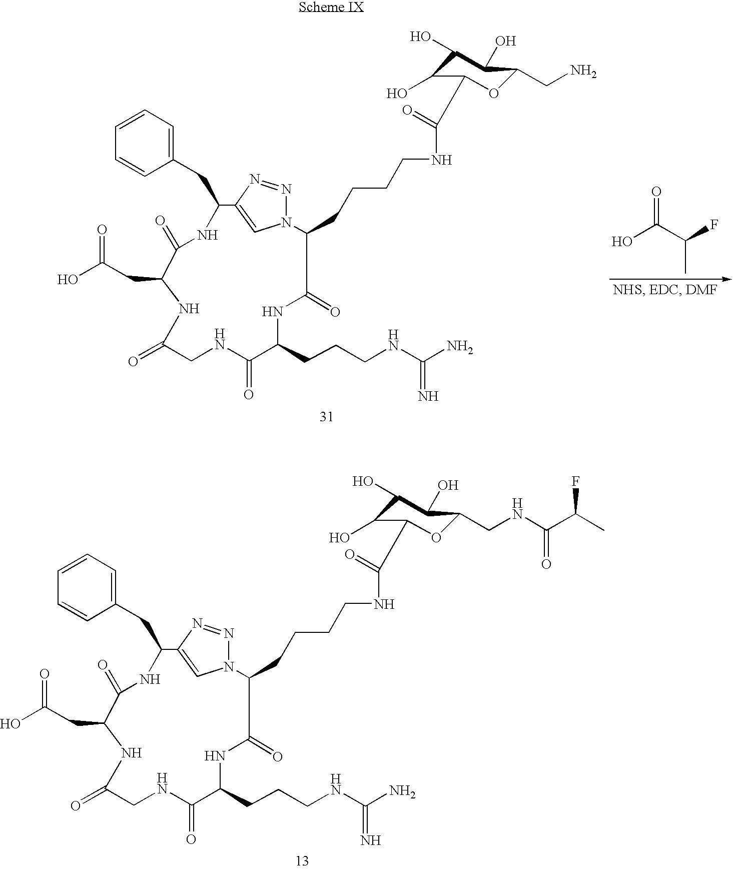

### Synthesis of Compound 13:

2-Fluoropropanoic acid (1.26 mg, 0.014 mmol) was dissolved in DMF (1 mL) and treated with NHS (1.58 mg, 0.014 mmol) and EDC (2.63 mg, 0.014 mmol) at room temperature. After stirring for 0.5 hr, a solution of compound 31 (3 mg, 3.43 μmol) in DMF (1 mL) and DIPEA (10 μL, 0.06 mmol) were added to the reaction mixture and stirred for 6 hr. LC/MS shows all the starting material was consumed. Solvent was removed under high vacuum, and residue was dissolved in water (3 mL). After filtration, the desired product was isolated by semi-preparative HPLC. The collected fractions were combined and lyophilized to afford compound 13 (1.7 mg, 53%) as a white fluffy powder. MS (m/z) (ESI): 891.3 [M+H]⁺, 913.3 [M+Na]⁺.

Another exemplary preparation of one of the cyclic peptidomiretics derivatives of the present application, Compound 14, is shown in Scheme X

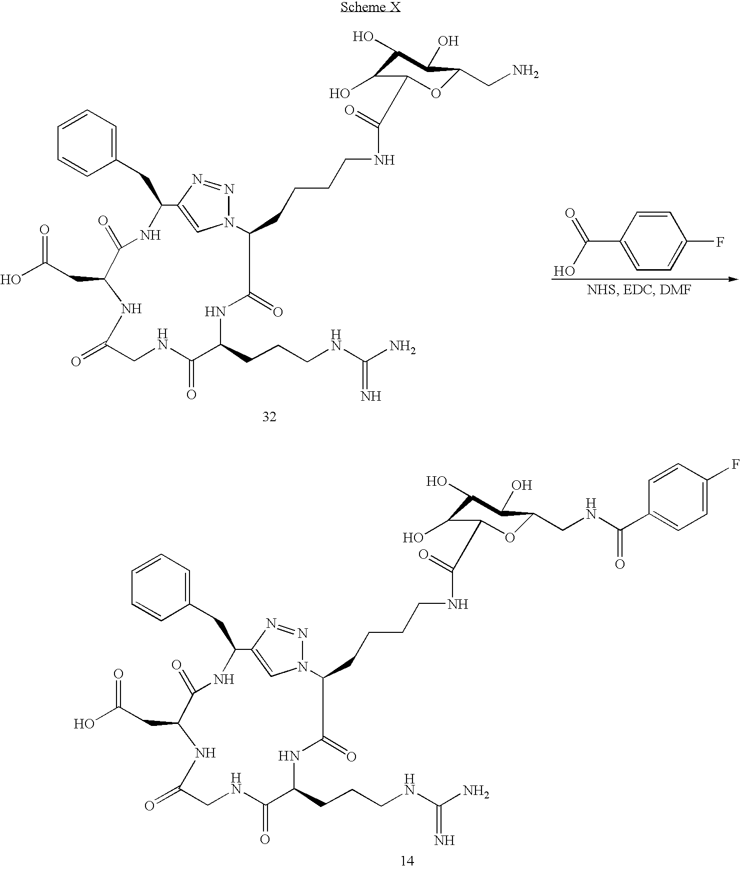

### Synthesis of Compound 14:

4-Fluorobenzoic acid (1 mg, 6.89 nmol) was dissolved in DMF (0.5 mL) and treated with NHS (1 mg, 6.89 nmol) and EDC (1.32 mg, 6.89 nmol) at room temperature. After stirring for 0.5 hr, a solution of compound 31 (4.5 mg, 5.51 μmol) in DMF (0.5 mL) and DIPEA (10 μL, 0.06 mmol) were added to the reaction mixture and stirred for 3 hr. LC/MS shows all the starting material was consumed. Solvent was removed under high vacuum, and residue was dissolved in water (3 mL). After filtration, the desired product was isolated by semi-preparative HPLC. The collected fractions were combined and lyophilized to afford compound 14 (3 mg, 56%) as a white fluffy powder. MS (m/z) (ESI): 939.4 [M+H]+, 961.4 [M+Na]+.

### Radiosynthesis

The radiolabeling methods for different cyclic peptidomimetics are listed in Table 1. Cu(I) catalyzed ‘click chemistry’ is used to prepare most of ¹⁸F-radiolabeled RGD cyclic peptidomimetics. The [¹⁸F]-fluoroalkyne is prepared using corresponding tosylated alkyne as precursor. Conjugation of [¹⁸F]fluoroalkyne to cyclopeptides or cyclic peptidomimetics derivatized with azido group via Cu(I) mediated 1,3-dipolar cycloaddition yields the desired ¹⁸F-labeled products with good yields and excellent radiochemical purity. An exemplary preparation of one of the ¹⁸F-radiolabeled cyclic peptidomimetics using click chemistry approach, Compound 2, is shown in Scheme XI.

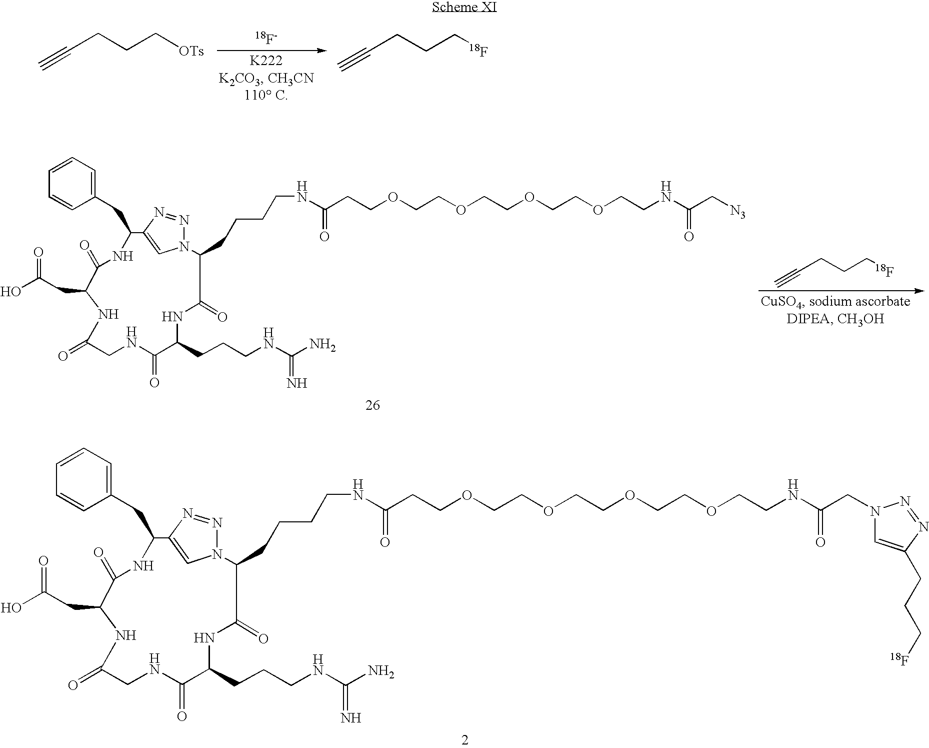

### Synthesis of [¹⁸F]-Compound 2

1-Pentynyl tosylate (15˜18 mg) is ¹⁸F-labeled in CH₃CN at 110° C. in the presence of K222 and K₂CO₃ for 5 min while simultaneously distilling the material into a cooled solution containing 1˜2 mg of compound 26, 250 μL of CuSO₄ solution (0.1 M), 25 mg of sodium ascorbate, 250 μL of CH₃OH, and 50 μL DIPEA. The reaction is stirred for 45˜60 min at room temperature. The reaction mixture is then loaded onto an HPLC C18 column for purification. After collecting the product, the material is reconstituted via C18 loading and unloading with EtOH and diluting with water to make a 10% EtOH: Water solution. The yields vary from ˜35 mCi to ˜1 mCi.

### In Vitro Binding Assay:

TABLE 2
RGDfK derivatives employed in in vitro assay

| Compound | Chemical Structure | MW |
|---|---|---|
| 17 | 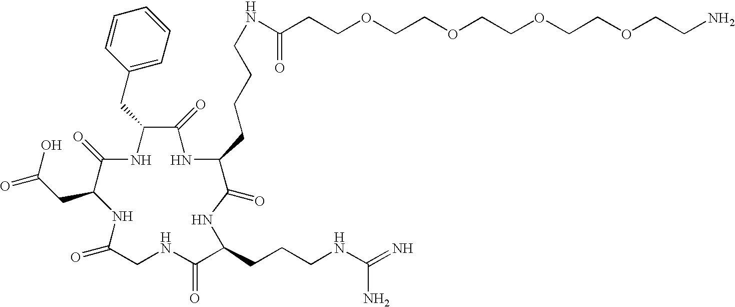 | 850.45 |
| 18 | 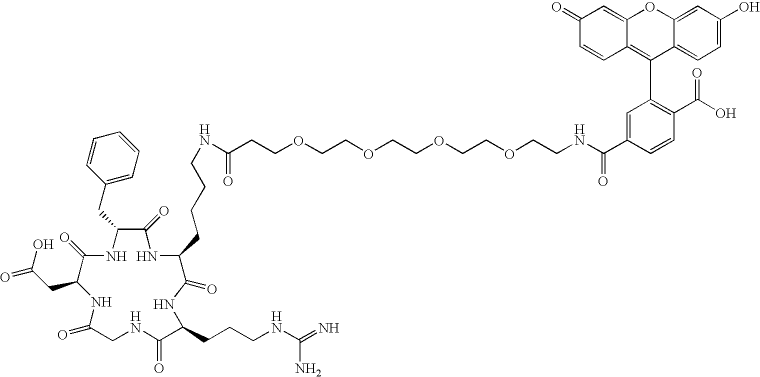 | 1208.50 |

### Surface Plasmon Resonance (SPR) Assay:

Compound 17 was immobilized onto a CM5 chip (Supplier: Biacore. CM5 is a SPR chip with a carboxymethylated dextran covalently attached to a gold surface) via amine coupling. Integrin αᵥβ₃ samples at 25 nM concentration, premixed with a wide range of concentrations of RGD test compound (0˜1000 nm), were flowed through the CM5 chip at 14° C. The interactions between the flowing integrin αᵥβ₃ sample and the surface of the chip were recorded by Biacore sensorgram signals. Flow cell #1 served as blank control and the flow cell #2 were coated with compound 17. After subtraction the blank signal of flow cell #1 from the signal of flow cell #2, the resulting sensorgram signals from each cycle were converted into percentage values. Then the K_{d} and IC₅₀ values for each RGD compounds were calculated.

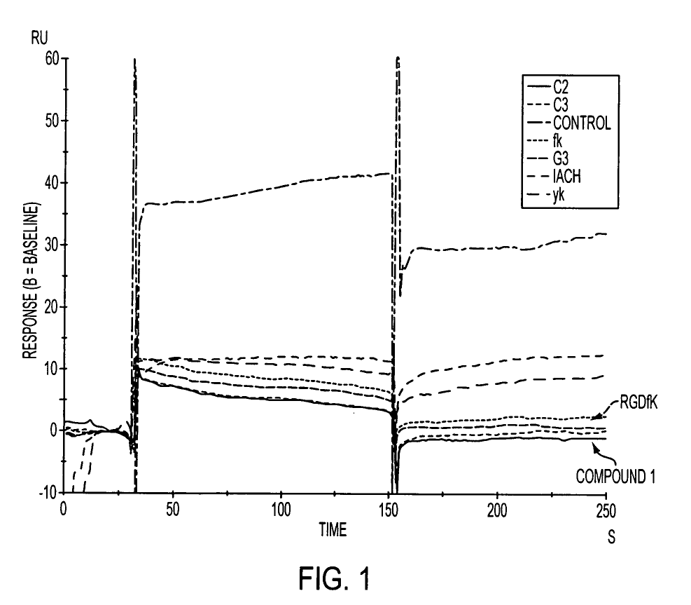

The results of this ‘inverse’ integrin αᵥβ₃ SPR assay show that compound 1 displays surprisingly high binding affinity to integrin αᵥβ₃. The K_{d} and IC₅₀ values of compound 1 are very close to those of RGDfK, a well-known inhibitor to integrin αᵥβ₃. See FIG. 1.

### Cell-Based Integrin Binding Competition Assay:

Integrin αᵥβ₃ expressing U87MG cells were incubated with a series of concentration of RGD compounds (0-32 μM) in the presence of 2 μM of green fluorescence labeled compound 18 for 2 hrs. After incubation, cells were washed three times to eliminate unbound RGD compounds. Fluorescence readings (RLU) were then taken (excitation at 491 nm, emission at 518 nm, cutoff 515 nm).

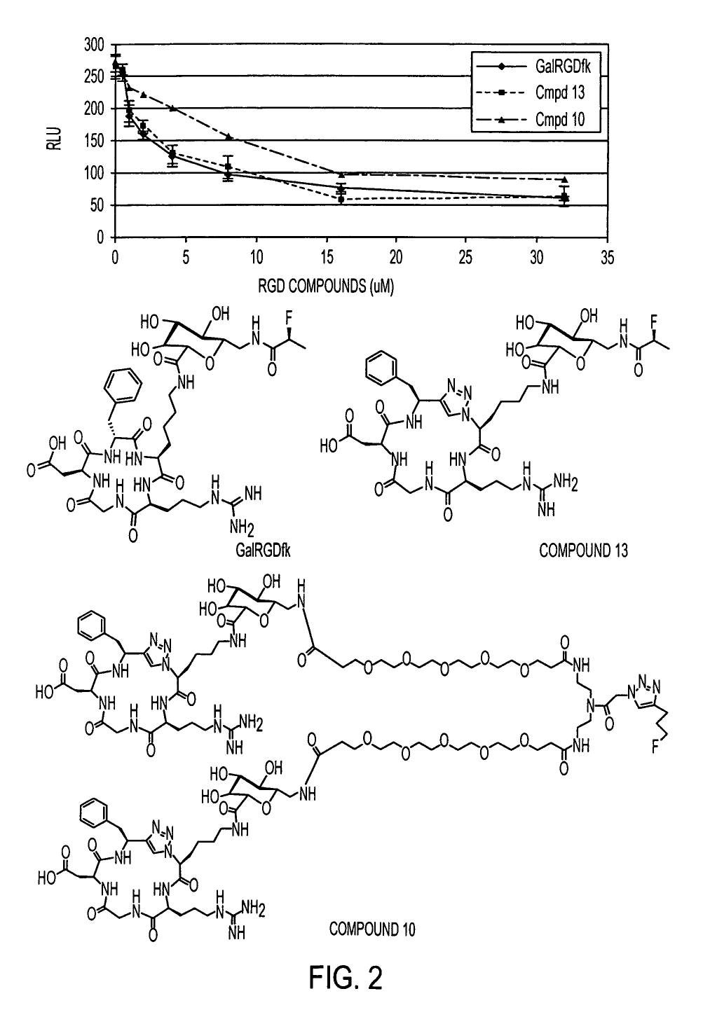

The results are consistent with that of surface plasmon resonance assay. The data further demonstrate that compound 1 and RGDFK are very similar in potency. See FIG. 2.

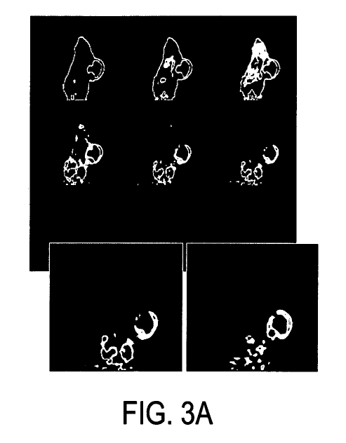

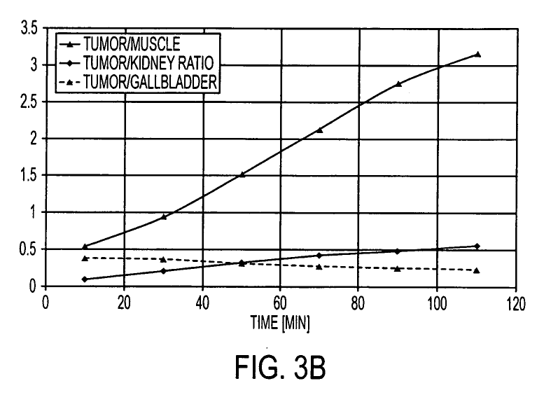

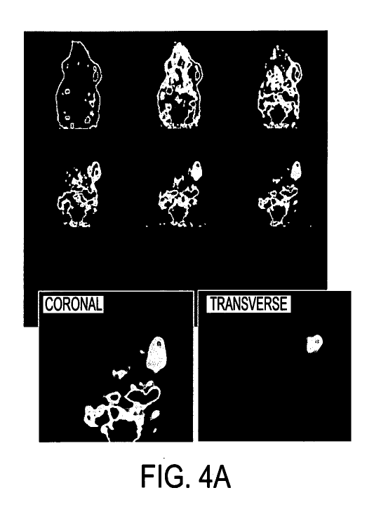

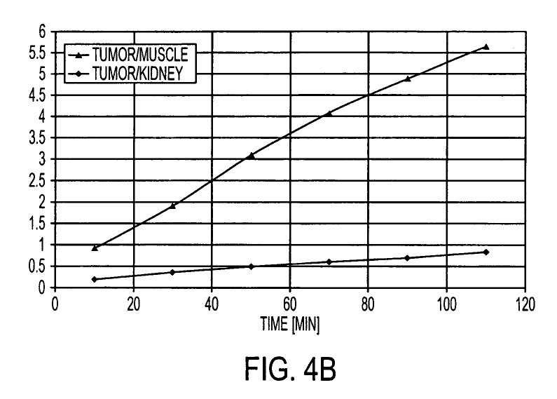

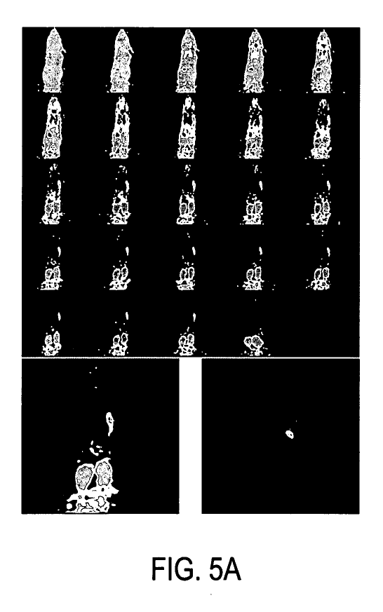

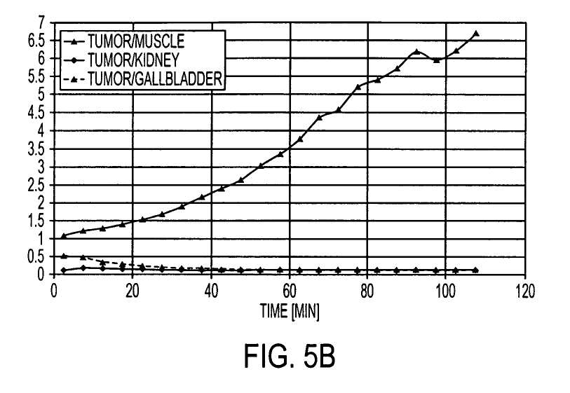

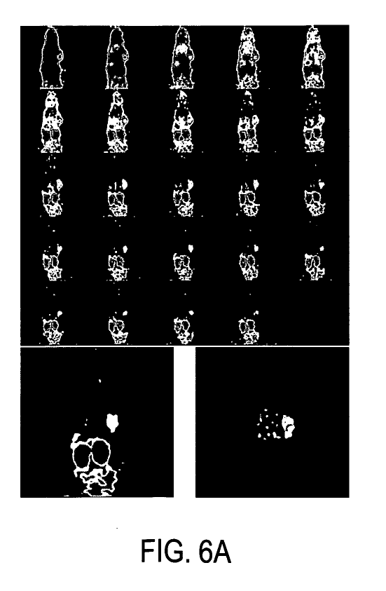

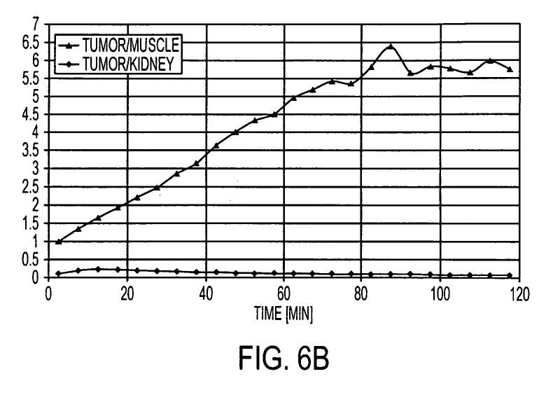

PET Studies: In vivo microPET imaging of a tumor-bearing mouse is performed on an anesthetized mouse bearing tumor xenograft of either U87MG human glioblastoma or A431 human squamous cell carcinoma after administration of cyclic peptidomimetic. In vivo microPET imaging shows that compound 2 and compound 3 are very good tracers with a) good tumor uptake and retention, b) favorable renal clearance and very little liver uptake, c) fast wash-out rate from muscle and other healthy tissues, which includes kidney. See e.g. FIG. 3-6.

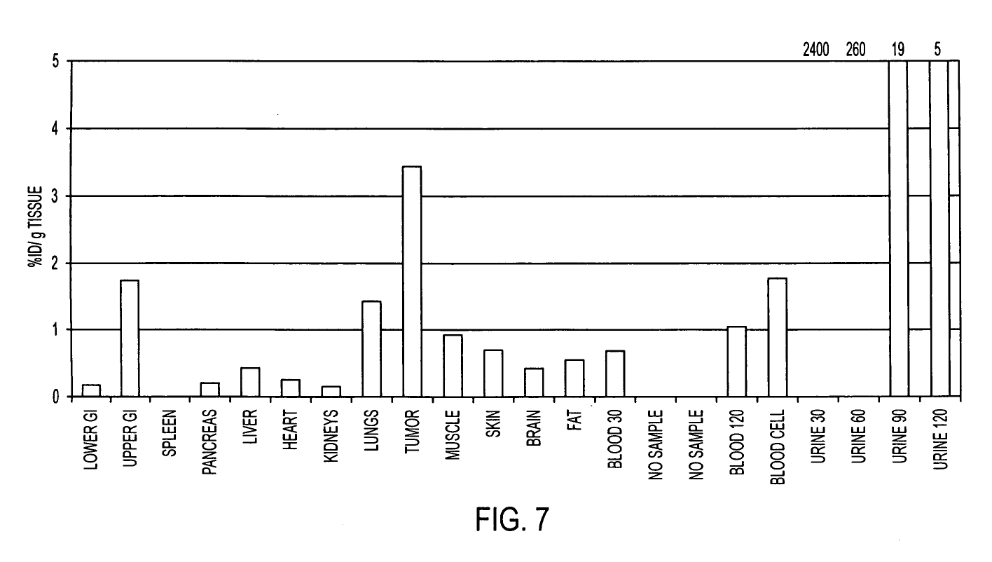

Biodistribution Studies: Nude mice bearing tumor xenograft of U87MG human glioblastoma are i.v. injected with compound 2. The animals are sacrificed and dissected at fixed times after injection. The major organs and fluids, including blood, muscle, gall bladder, liver, and tumor are removed and weighed. The amount of compound in the tissue is measured using LC/MS. Results are expressed as % ID/g (% Injected Dose/gram). See FIG. 7.

Metabolic Stability Studies for Compound 2 and Compound 3: For each tracer (radiolabeled compound): Two mice were anesthetized with Florane. For each mouse, 300 μCi of tracer in 200 μL saline was injected into the tail vein. Pressure was applied to the injection site for one minute to stop bleeding. The mice were then placed in a clean cage (one mouse/cage) without any bedding and observed until it is awaken.

In order to confirm the elution time of the radiolabeled compounds 2 and 3, the tracer (2 μL) and the corresponding unlabeled compound (‘the cold standard’) (dissolved in 200 μL water) were co-injected into radio-HPLC. In each case, the retention time of the tracer as determined by the radiodetector was identical to the retention time of the cold standard compound as determined by the UV detector.

At 30 or 60 minutes post injection, 300-500 μL of blood was drawn via cardiac puncture into a syringe containing anti-coagulant. The blood was then centrifuged for 3 minutes to separate plasma. The mice were then killed and the liver containing the gall bladder and kidneys were harvested and placed into separate tubes containing 2 mL lysis buffer. They were homogenized mechanically. 400 μL of each homogenate was then transferred to a tube, extracted with 200 μL chloroform/methanol (50/50) mixture, and vertexed.

All urine was collected from each cage at 30 or 60 minutes time point. 10 mL of water was then added to wash the dried urine in the cages. 1 mL of solution was taken out and transferred into a tube. The radioactivity was then measured using gamma counter.

Lysis buffer and chloroform/methanol mixture was also added to plasma and urine samples after they were weighted (sample weight in gram). All tubes were vortexed and frozen in dry ice. After the tubes were centrifuged for 3 minute, supernatant was transferred into new tubes. The radioactivity in the supernatant and precipitation were counted at the same time to calculate total injected dose. The sample CPM is the sum of CPM in the supernatant and in the precipitation. Thus, the percentage of injected dose per tissue weight (gram) can be calculated according to the following function:

% injected dose/g tissue=sample CPM/sample weigh (g)/(2 μl CPM×100).

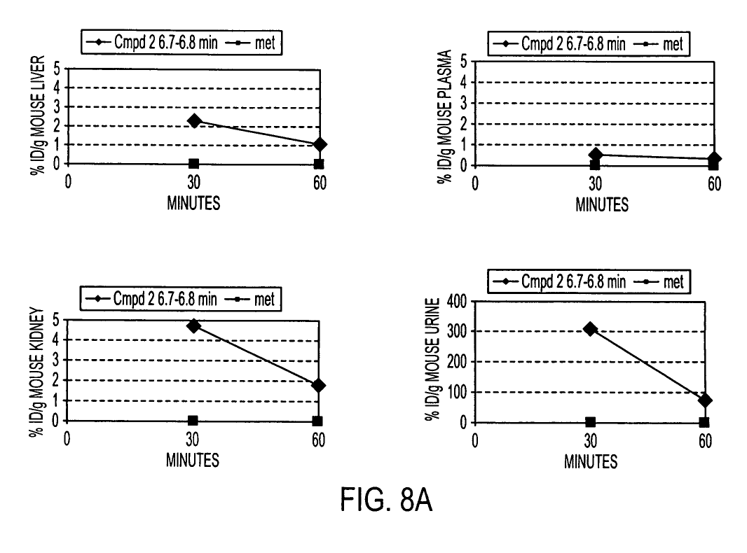

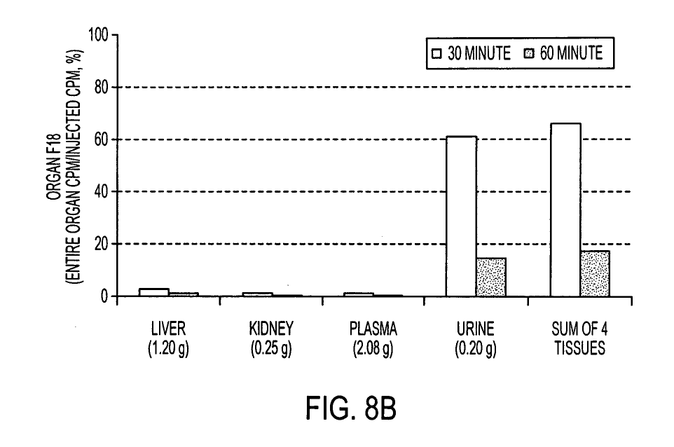

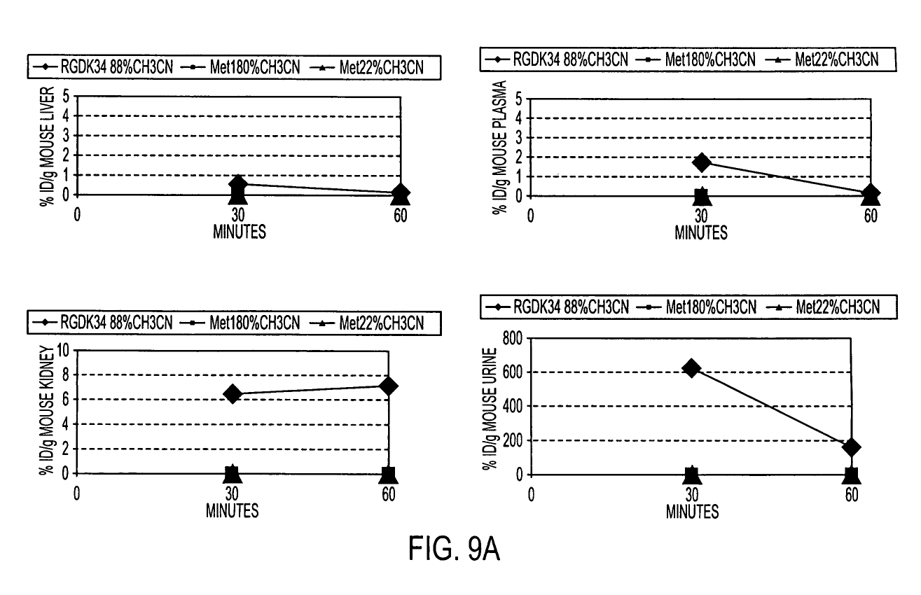

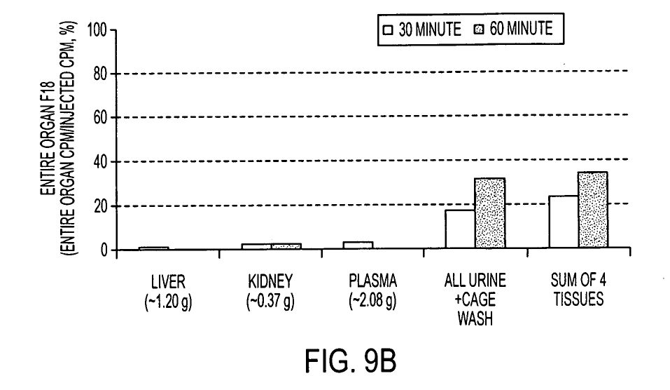

While there are metabolites in the body, the percentage of the original tracer and that of metabolites can be calculated from the radio-HPLC data. The data shows that only minor amounts radioactive metabolites found in the tissue and fluid samples for [¹⁸F]-radiolabeled compound 2 and 3. Thus, [¹⁸F]-radiolabeled compound 2 and 3 are very stable in mouse body. See e.g. FIGS. 8 and 9.

All references cited herein are incorporated by reference as if each had been individually incorporated by reference in its entirety. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

### REFERENCES

1. Aumailley, M.; Gurrath, M.; Muller, G.; Calvete, J.; Timpl, R.; Kessler, H. FEBS Lett. 1991, 291, 50-54.

2. Chen, X.; Park, R.; Shahinian, A. H.; Bading, J. R.; Conti, P. S. Nucl. Med. Biol., 2004, 31, 11-19.

3. Chen, X.; Park, R.; Hou, Y.; Khankaldyyan, V.; Gonzales-Gomez, I.; Tohme, M.; et al. Eur. J. Nul. Med. Mol. Imaging, 2004, 31, 1081-1089.

4. Chen, X.; Hou, Y.; Tohme, M.; Park, R.; Khankaldyyan, V.; Gonzales-Gomez, I.; et al. J. Nul. Med., 2004, 45, 1776-1783.

5. Haubner, R.; Weber; W. A.; Beer, A. J.; Vabuliene, E.; Reim, D.; Sarbia, M.; Becker, K. F.; Goebel, M., et al. PLoS Med., 2005, 2, e70.

6. Haubner, R.; Wester, H. J.; Weber, W. A.; Mang, C.; Ziegler, S. I.; Goodman, S. L.; Senekowisch-Schmidtke, R.; Kessler, H.; Schwaiger, M. Cancer Res., 2001, 61, 1781-1785.

7. Haubner, R.; Kuhnast B,; Mang, C.; Weber W. A.; Kessler, H.; Wester, H. J.; Schwaiger, M. Bioconjug. Chem., 2004, 15, 61-69.

8. Belvisi, L.; Bernardi, A.; Checchia, A.; et al. Org. Lett., 2001, 3, 1001-1004.

9. Kolb, H. C.; Finn, M. G.; Sharpless, K. B. Angewandte Chemie, International Edition 2001, 40, 2004-2021.

10. Kolb, H. C.; Sharpless, K. B. Drug Discovery Today 2003, 8, 1128-1137.

11. Rostovtsev, V. V.; Green, L. G.; Fokin, V. V.; Sharpless, K. B. Angewandte Chemie, International Edition 2002, 41, 2596-2599.

12. Tornøe, C. W.; Christensen, C.; Meldal, M. Journal of Organic Chemistry 2002, 67, 3057-3064.

13. Wang, Q.; Chan, T. R.; Hilgraf, R.; Fokin, V. V.; Sharpless, K. B.; Finn, M. G. Journal of the American Chemical Society 2003, 125, 3192-3193.

14. Lee, L. V.; Mitchell, M. L.; Huang, S. J.; Fokin, V. V.; Sharpless, K. B.; Wong, C. H. Journal of the American Chemical Society 2003, 125, 9588-9589.

15. Lewis, W. G.; Green, L. G.; Grynszpan, F.; Radic, Z.; Carlier, P. R.; Taylor, P.; Finn, M. G.; Barry, K. Angew. Chem., Int. Ed. 2002, 41, 1053-1057.

16. Manetsch, R.; Krasinski, A.; Radic, Z.; Raushel, J.; Taylor, P.; Sharpless, K. B.; Kolb, H. C. Journal of the American Chemical Society 2004, 126, 12809-12818.

17. Mocharla, V. P.; Colasson, B.; Lee, L. V.; Roeper, S.; Sharpless, K. B.; Wong, C. H.; Kolb, H. C. Angew. Chem. Int. Ed. 2005, 44, 116-120.

18. Beer, A. J., et al., J. Nucl. Med. 2006 47:763-769.

What is claimed is:

1. A peptidomimetic of formula I: 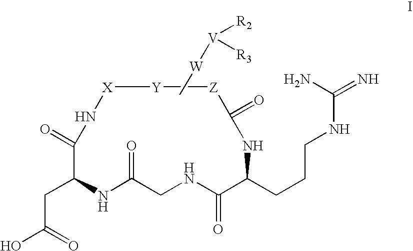
wherein
W is a 5- or 6-membered heterocycle or a linker comprising a hydrophilic moiety selected from the group consisting of hydrokyl, carbonyl, sulfonamide, sulfonate, phosphate, polar amino acid moiety, PEG moiety, sugar mimetic, and sugar moiety;
V is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;
wherein at least one, but not both of W and V is a 5- or 6-membered heterocycle;
X is selected from the group consisting of —C₁-C₆ alkyl-(5-to 6-membered heterocycle)-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;
Y is selected from the group consisting of 5- or 6-membered heterocycle, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;
Z is selected from the group consisting of -(5- or 6-membered heterocycle)—C₁-C₆ alkyl-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;
any one of X, Y, or Z but not more than one of X, Y and Z is a 5- or 6-membered heterocycle;
where each R₁ is independently selected from the group consisting of a side chain of a natural amino acid and a side chain of an unnatural amino acid, wherein the natural amino acid and the unnatural amino acid is either in the D or L form;
R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene, carbocycle and heterocycle groups are each optionally substituted; and
optionally the fragment W—V(R₂)(R₃) is absent;
wherein at least one of W, X, Y, Z, R₂, and R₃ comprises a radionuclide selected from the group consisting of positron or gamma emitters.

2. The peptidomimetic of claim 1, wherein:
Y is a 5-membered heterocycle;
V is a 5-membered heterocycle;
each of X and Z is a linker selected from the group consisting of comprising —C(H)(R₁)—, and optionally substituted C₁-C₆ alkyl; and
the radionuclide is selected from the group consisting of ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, ¹³¹I, ⁹⁹Tc, ⁷⁵Br, ¹⁵³Gd and ³²P.

3. The peptidomimetic of claim 2 wherein:
W is selected from the group consisting of: 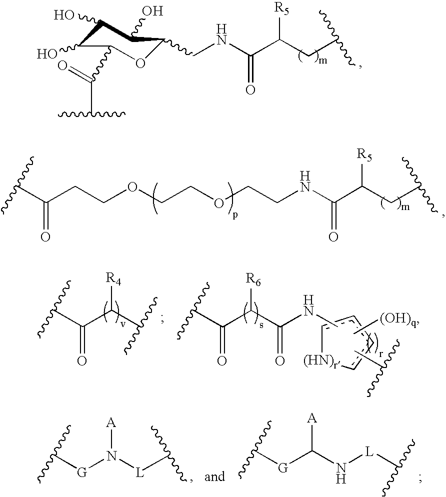
where R₄ is selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, C₁-C₆-alkoxy-C₁-C₆-alkyl, and a PEG moiety, wherein the alkyl, alkenyl, alkynyl, alkyloxy, aryl, carbocycle, and heterocycle groups are each optionally substituted;
R₅ is selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, and C₁-C₆-alkoxy-C₁-C₆-alkyl, wherein the alkyl, alkenyl, alkynyl, alkyloxy, aryl, carbocycle, and heterocycle groups are each optionally substituted;
each R₆ is independently selected from the group consisting of —H, —OH, C₁-C₆ alkyl, C₂-C₆ alkenyl, C₂-C₆ alkynyl, C₁-C₆ alkyloxy, aryl-(C₁-C₆ alkylene)-, hydroxy-C₁-C₆-alkyl, and C₁-C₆-alkoxy-C₁-C₆-alkyl, wherein the alkyl, alkenyl, alkynyl, alkyloxy, and aryl-alkylene groups are each optionally substituted;
G is selected from the group consisting of: 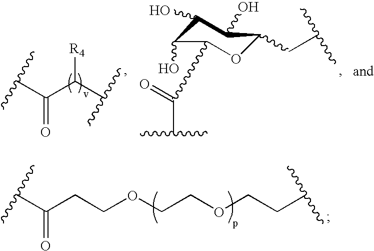
L is selected from the group consisting of: 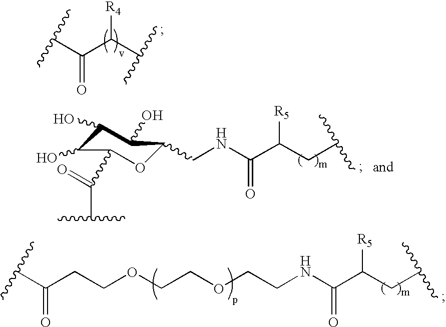
A is selected from the group consisting of: 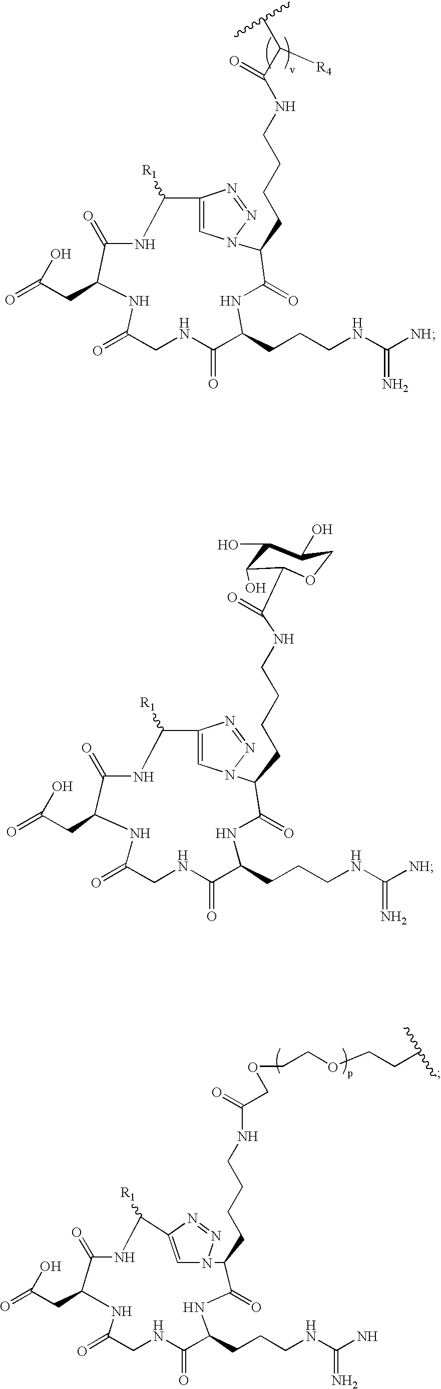 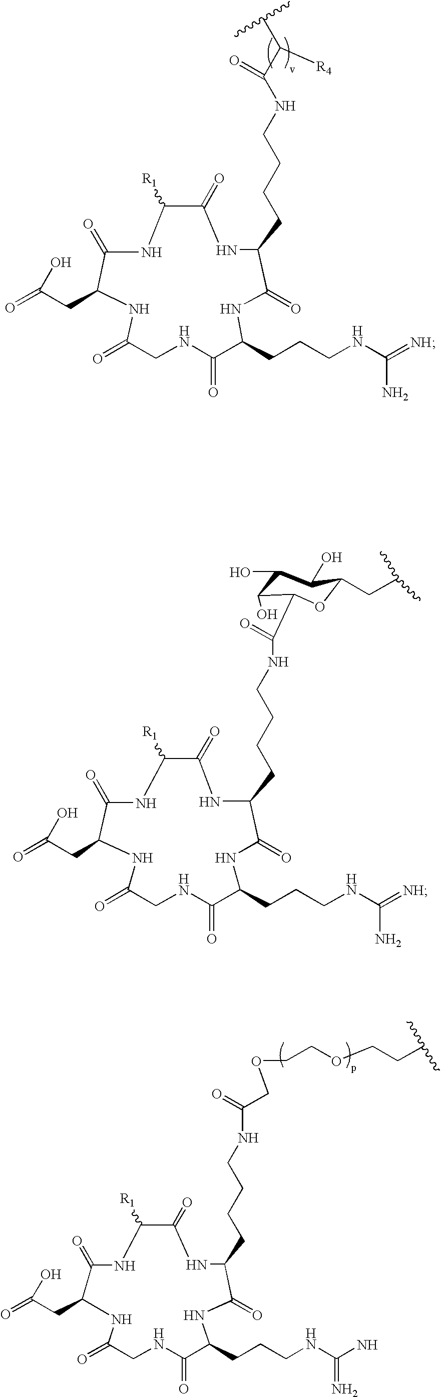
where R₁ is selected from the group consisting of a side chain of a natural amino acid and a side chain of an unnatural amino acid, wherein the natural amino acid and the unnatural amino acid is either in the D or L form;
each v is 0, 1, 2, 3, or 4;
m is 0, 1, 2, 3 or 4;
p is an integer between 1 and 110;
q is 1, 2, 3 or 4;
r is 1, 2 or 3;
r′ is 0 or 1; and
s is 1, 2, 3 or 4;
wherein the configuration of the chiral centers may be R or S or mixtures thereof.

4. The peptidomimetic of claim 3 wherein:
R₁ is a side chain of a natural amino acid;
W is 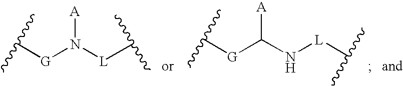
V is 1,2,3-triazolyl;
R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I and ¹³¹I.

5. The peptidomimetic of claim 4 wherein:
W is 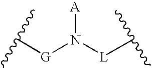
Where G is 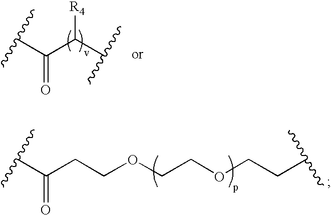
L is 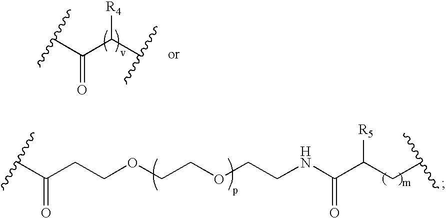
where m is 0 or 1;
p is an integer between 1 and 25;
vis 0, 1, or 2.

6. The peptidomimetic of claim 5 wherein:
G is 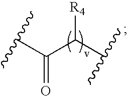
and A is 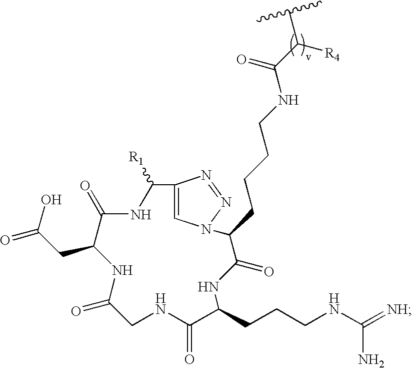
where each R₄ is independently selected from the group consisting of —H and optionally substituted C₁-C₆ alkyl; and each v is 1 or 2.

7. The peptidomimetic of claim 4 wherein:
W is 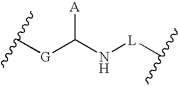
where G is 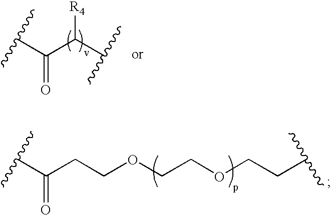
L is 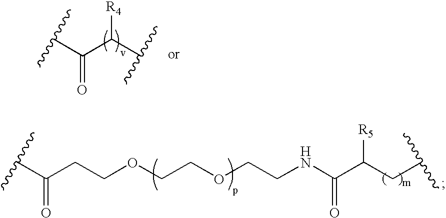
where m is 0 or 1;
p is an integer between 1 and 25;
v is 0, 1, or 2.

8. A peptidomimetic of formula II 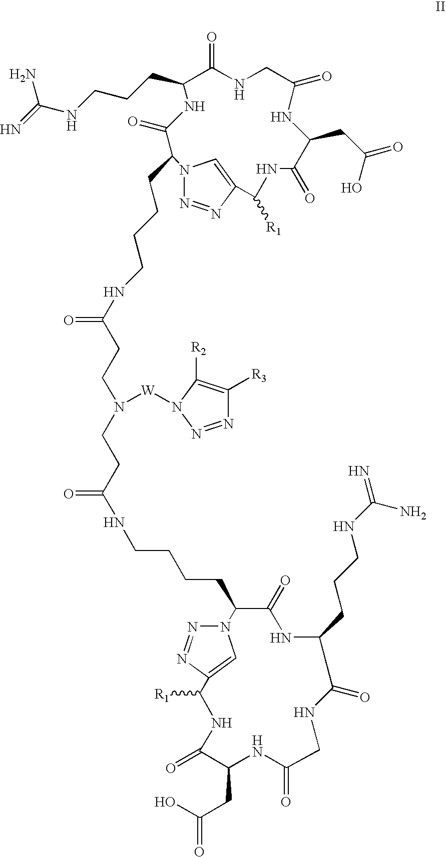
wherein:
each R₁ is independently selected from the group consisting of a side chain of a natural amino acid and a side chain of an unnatural amino acid, wherein the natural amino acid and the unnatural amino acid is either in the D or L form;
R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I and ¹³¹I.
W is selected from the group consisting of: 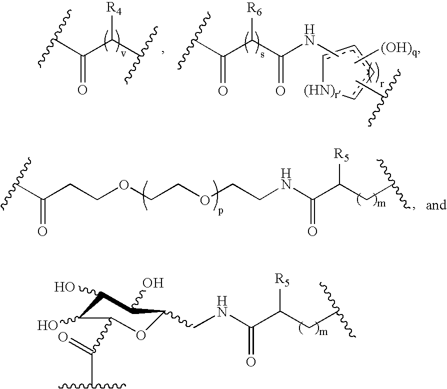
where p is 0 to 15;
v is 0, 1, 2, or 3;
m is 0, 1 or 2;
q is 1 or 2;
r is 1, 2 or 3;
r′ is 0 or 1; and
s is 1, 2, 3 or 4;
each R₄ and R₅ is independently selected from the group consisting of —H, and optionally substituted C₁-C₆ alkyl;
each R₆ is independently selected from the group consisting of —H, —OH, and optionally substituted C₁-C₆ alkyl;
wherein the configuration of the chiral center that carries the R₅ substituent may be R or S or mixtures thereof.

9. The peptidomimetic of claim 8 wherein:
W is 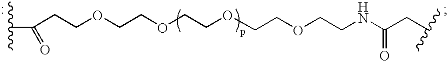
R₃ is —(CH₂)ₙ—¹⁸F; and R₂ is H;
where p is 0, 1, 2, 3, 4 or 5; and n is 1, 2, 3, 4 or 5.

10. The peptidomimetic of claim 9 wherein p is 0 and n is 3.

11. A peptidomimetic of formula III: 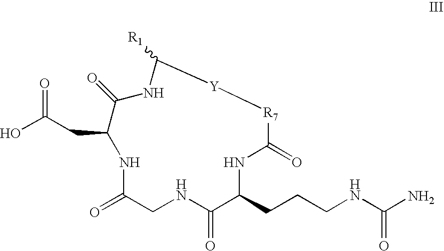
wherein:
Y is a 5 or 6 membered heterocycle; and
R₇ is selected from the group consisting of—C(H)(R₁)—, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene-, carbocycle and heterocycle groups are each optionally substituted;
each R₁ is independently selected from the group consisting of a side chain of a natural amino acid and a side chain of an unnatural amino acid, wherein the natural amino acid and the unnatural amino acid is either in the D or L form.

12. The peptidomimetic of claim 11 wherein Y is a 1,2,3-triazolyl; R₁ is benzyl; R₇ —C(H)(R₁)—.

13. A peptidomimetic of claim 11 of formula IIIB:

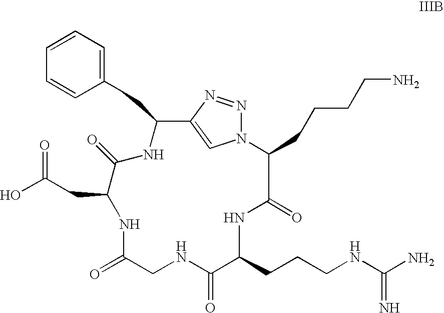

14. A peptidomimetic of formula IV: 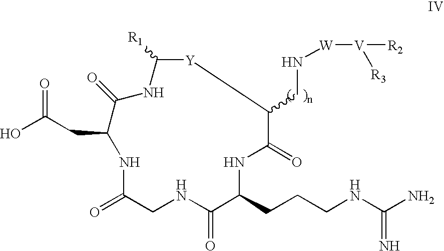
wherein
n is 0, 1, 2, 3, or 4;
R₁ is a selected from the group consisting of a side chain of natural amino acids and unnatural amino acids, wherein the natural amino acids and unnatural amino acids are either in the D or L form;
Y and V is each independently selected from a group consisting of 5 membered heterocycles and 6 membered heterocycles;
W is a linker comprising a hydrophilic moiety selected from the group consisting of hydroxyl, carbonyl, sulfonamide, sulfonate, phosphate, polar amino acid moiety, PEG moiety, sugar mimetic, and sugar moiety.
R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene, carbocycle and heterocycle groups are optionally substituted; wherein R₂ and R₃ are not both H;
wherein the configuration of the chiral centers may be R or S or mixtures thereof; and
either R₂ or R₃, or both R₂ and R₃ comprise a radionuclide selected from the group consisting of positron or gamma emitters.

15. The peptidomimetic of claim 14 wherein V is 1,2,3-triazolyl and n is 4.

16. The peptidomimetic of claim 14 wherein:
R₁ is a side chain of a natural amino acid; 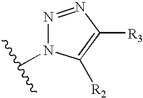
V is
W is selected from the group consisting of: 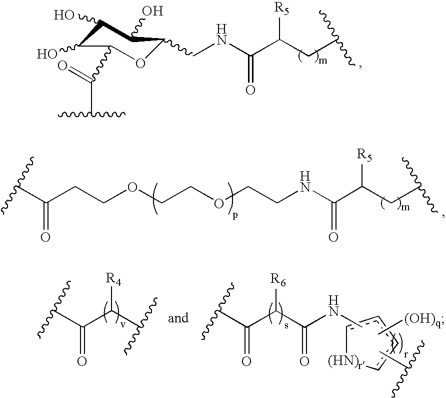
where R₄ is selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, C₁-C₆-alkoxy-C₁-C₆-alkyl, and a PEG moiety, wherein the alkyl, alkenyl, alkynyl, alkyloxy, aryl, aryl-alkylene, carbocycle and heterocycle groups are each optionally substituted; wherein the configuration of the chiral centers may be R or S or mixtures thereof;
R₅ is selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, and C₁-C₆-alkoxy-C₁-C₆-alkyl, wherein the alkyl, alkenyl, alkynyl, alkyloxy, aryl, carbocycle, and heterocycle, groups are each optionally substituted;
each R₆ is independently selected from the group consisting of —H, —OH, C₁-C₆ alkyl, C₂-C₆ alkenyl, C₂-C₆ alkynyl, C₁-C₆ alkyloxy, aryl-(C₁-C₆ alkylene)-, hydroxy-C₁-₆-alkyl, and C₁-C₆-alkoxy-C₁-C₆-alkyl, wherein the alkyl, alkenyl, alkynyl, alkyloxy, and aryl-alkylene groups are each optionally substituted;
q is 1, 2, 3 or 4;
r is 1, 2 or 3;
r′ is 0 or 1; and
s is 1, 2, 3 or 4;
v is 0, 1, 2, 3, or 4;
m is 0, 1, 2, 3, or 4; and
p is an integer between 0 and 15;
wherein either R₂ or R₃, or both R₂ and R₃ comprise a radionuclide selected from the group consisting of ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, ¹³¹I, ⁹⁹Tc, ⁷⁵Br, ¹⁵³Gd and ³²P.

17. The peptidomimetic of claim 16 wherein:
W is 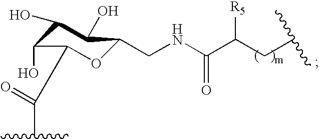
R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I and ¹³¹I;
R₅ is selected from the group consisting of —H, C₁-C₆ alkyl, C₂-C₆ alkenyl, and C₂-C₆ alkynyl, wherein the alkyl, alkenyl and alkynyl groups are each optionally substituted and wherein the configuration of the chiral center that carries the R₅ substituent may be R or S or mixtures thereof; and
m is 0, 1 or 2.

18. The peptidomimetic of claim 17, wherein:
R₂ is hydrogen;
R₃ is selected from the group consisting of C₁-C₄ alkyl, C₂-C₄ alkenyl, and C₂-C₄ alkynyl, wherein the alkyl, alkenyl and alkynyl groups are each optionally substituted, wherein R₃ comprises a radionuclide selected from the group consisting of ¹¹C, ¹³N, ¹⁵O, and ¹⁸F;
R₅ is hydrogen; and
m is 0.

19. The peptidomimetic of claim 16, wherein:
R₂ and R₃ are each independently selected from the group consisting of —H, C₁-C₆ alkyl, C₂-C₆ alkenyl, C₂-C₆ alkynyl, wherein the alkyl, alkenyl and alkynyl groups are each optionally substituted; wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I, and ¹³¹I;
W is 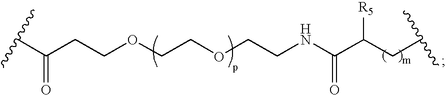
where R₅ is selected from the group consisting of —H, C₁-C₆ alkyl, C₂-C₆ alkenyl, and C₂-C₆ alkynyl, wherein the alkyl, alkenyl and alkynyl groups are each optionally substituted and wherein the configuration of the chiral center that carries the R₅ substituent may be R or S or mixtures thereof;
m is 0, 1, or 2; and
p is an integer between 1 and 90.

20. The peptidomimetic of claim 19, wherein:
R₂ is hydrogen;
R₃ is selected from the group consisting of C₁-C₄ alkyl, C₂-C₄ alkenyl, and C₂-C₄ alkynyl, wherein the alkyl, alkenyl and alkynyl groups are each optionally substituted, and R₃ comprises a radionuclide selected from the group consisting of ¹¹C, ¹³N, ¹⁵O, and ¹⁸F;
R₅ is hydrogen;
m is 0; and
p is an integer between 1 and 15.

21. The peptidomimetic of claim 16 wherein:
W is 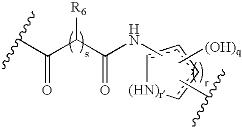
where each R₆ is independently selected from the group consisting of —H, —OH, C₁-C₆ alkyl, C₂-C₆ alkenyl, C₁-C₆ alkyloxy, hydroxy-C₁-C₆-alkyl, and C₁-C₆-alkoxy-C₁-C₆-alkyl, wherein the alkyl, alkenyl, and alkyloxy groups are each optionally substituted;
q is 2, 3 or 4;
r is 1, 2 or 3;
r′ is 0 or 1; and
s is 1 or 2.

22. The peptidomimetic of claim 21 wherein each R₆ is independently selected from the group consisting of —H, —OH and optionally substituted C₁-C₆ alkyl; q is 2; r is 2 or 3; and r′ is 0.

23. A peptidomimetic of formula V: 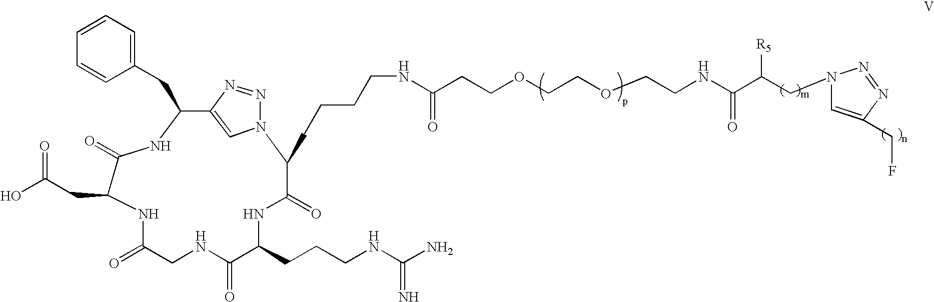
wherein R₅ is selected from the group consisting of —H, C₁-C₆ alkyl, C₂-C₆ alkenyl, C₂-C₆ alkynyl, C₁-C₆ alkyloxy, aryl, aryl-(C₁-C₆ alkylene)-, 3- to 7-membered carbocycle, 3- to 7-membered heterocycle, hydroxy-C₁-C₆-alkyl, and C₁-C₆-alkoxy-C₁-C₆-alkyl, wherein the alkyl, alkenyl, alkynyl, alkyloxy, aryl, aryl-alkylene, carbocycle, heterocycle, hydroxyalkyl and alkoxy-alkyl groups are each optionally substituted; p is an integer between 0 and 15;
m is 0, 1, 2, 3, or 4;
n is 1, 2, 3, 4, or 5; and
F is optionally a radionuclide;
wherein the configuration of the chiral centers may be R or S or mixtures thereof;

24. A peptidomimetic comprising of the formula:

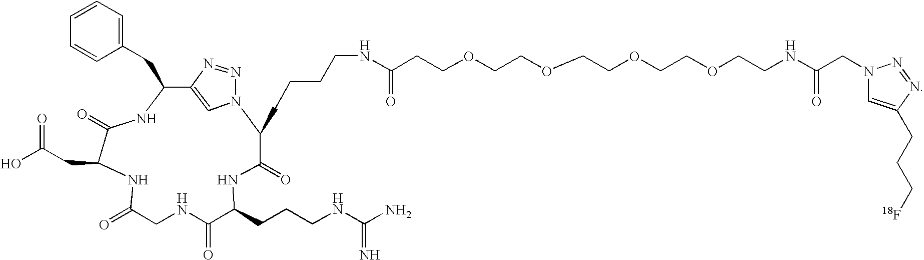

25. A peptidomimetic selected from the group consisting of:

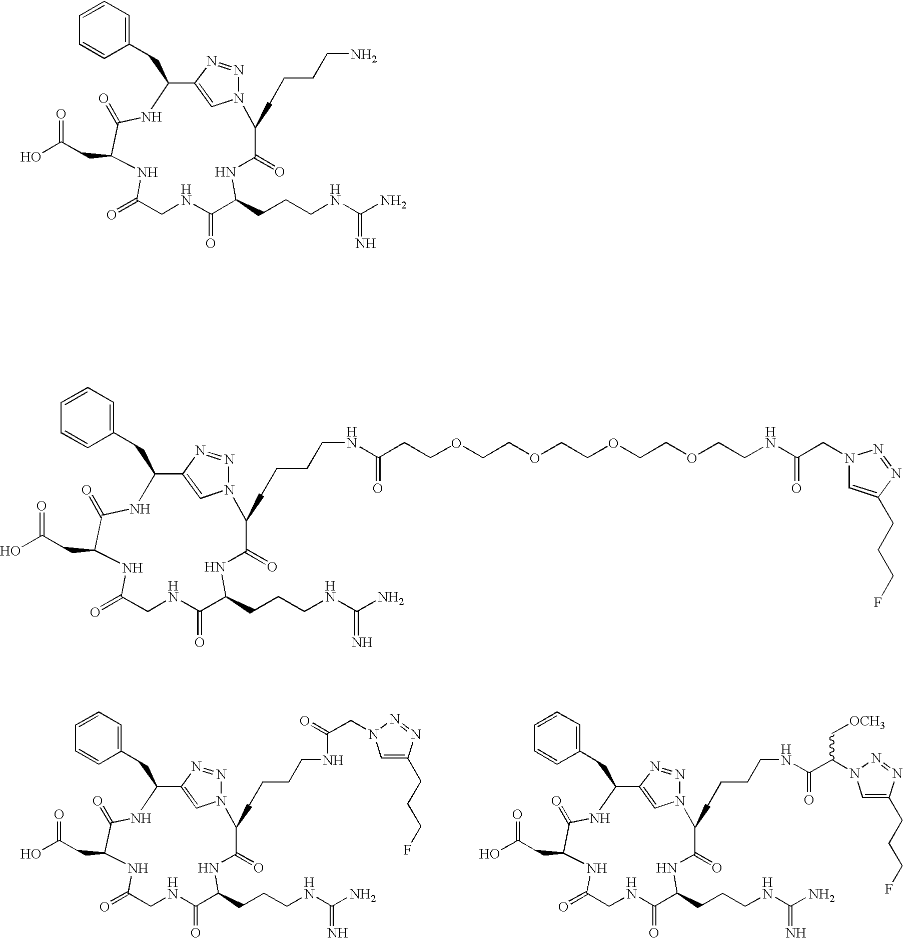

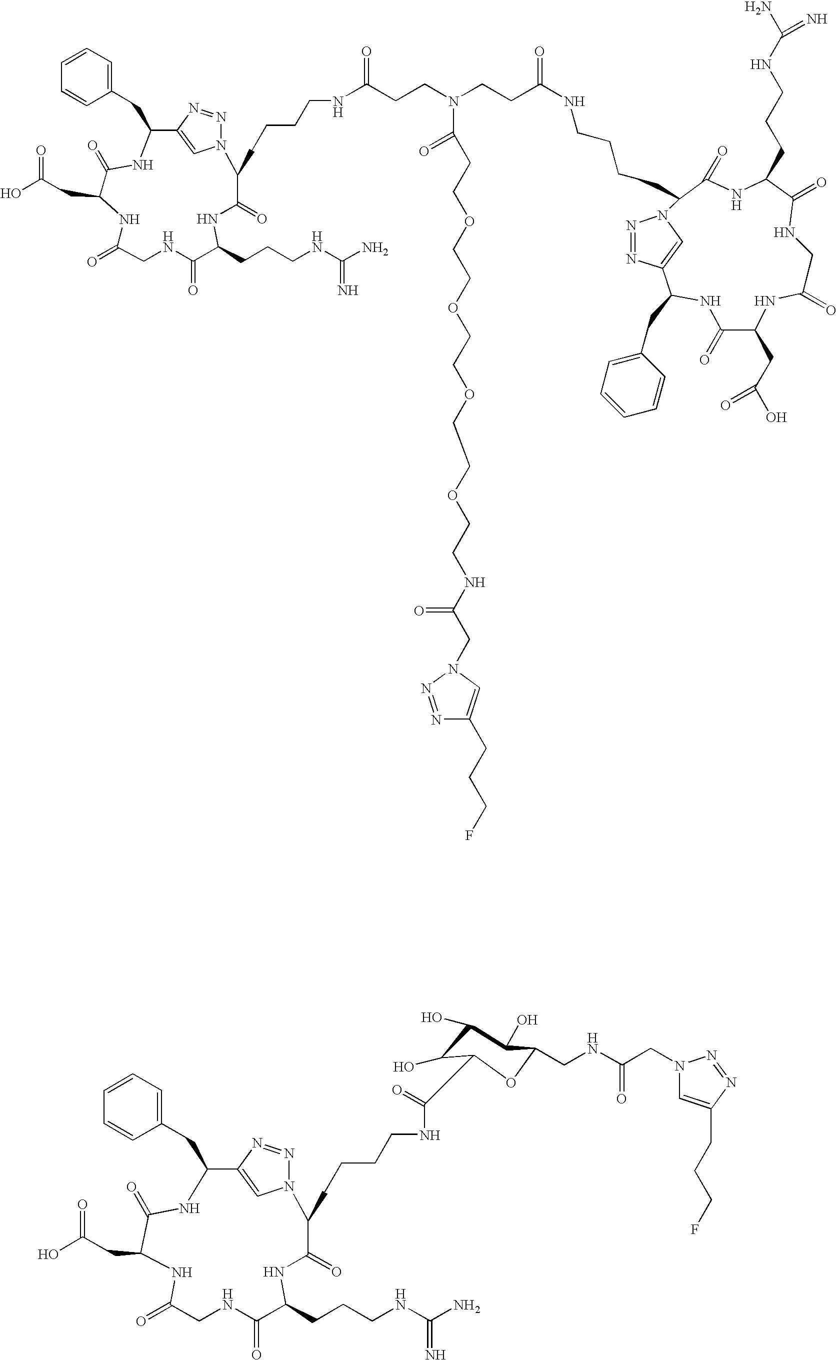

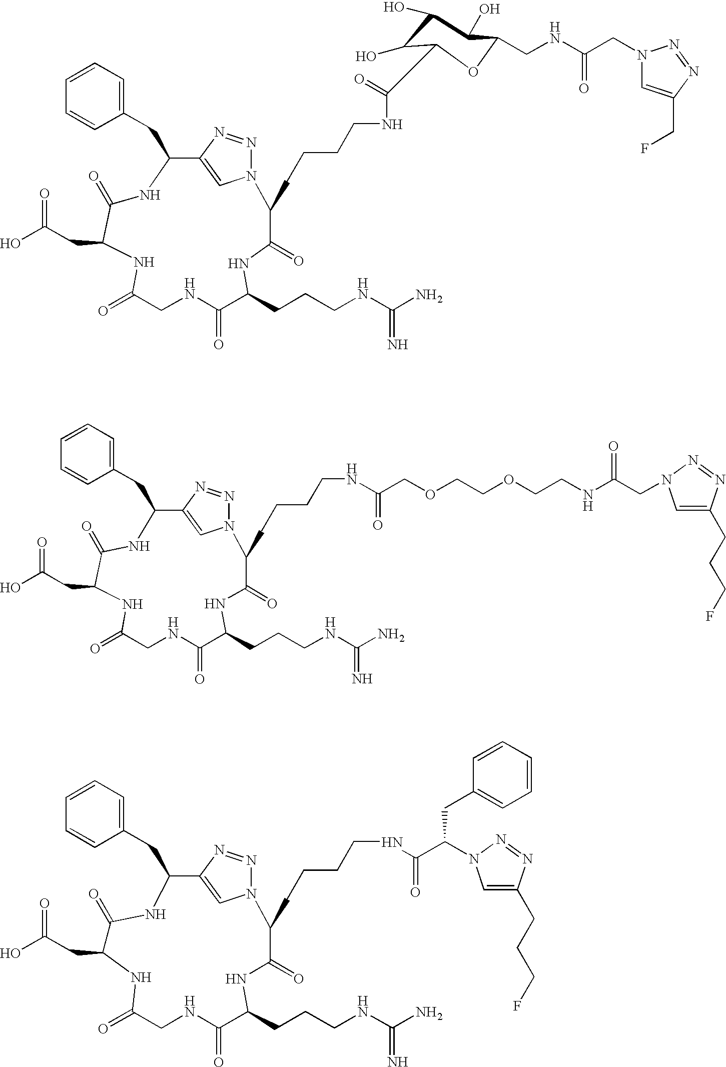

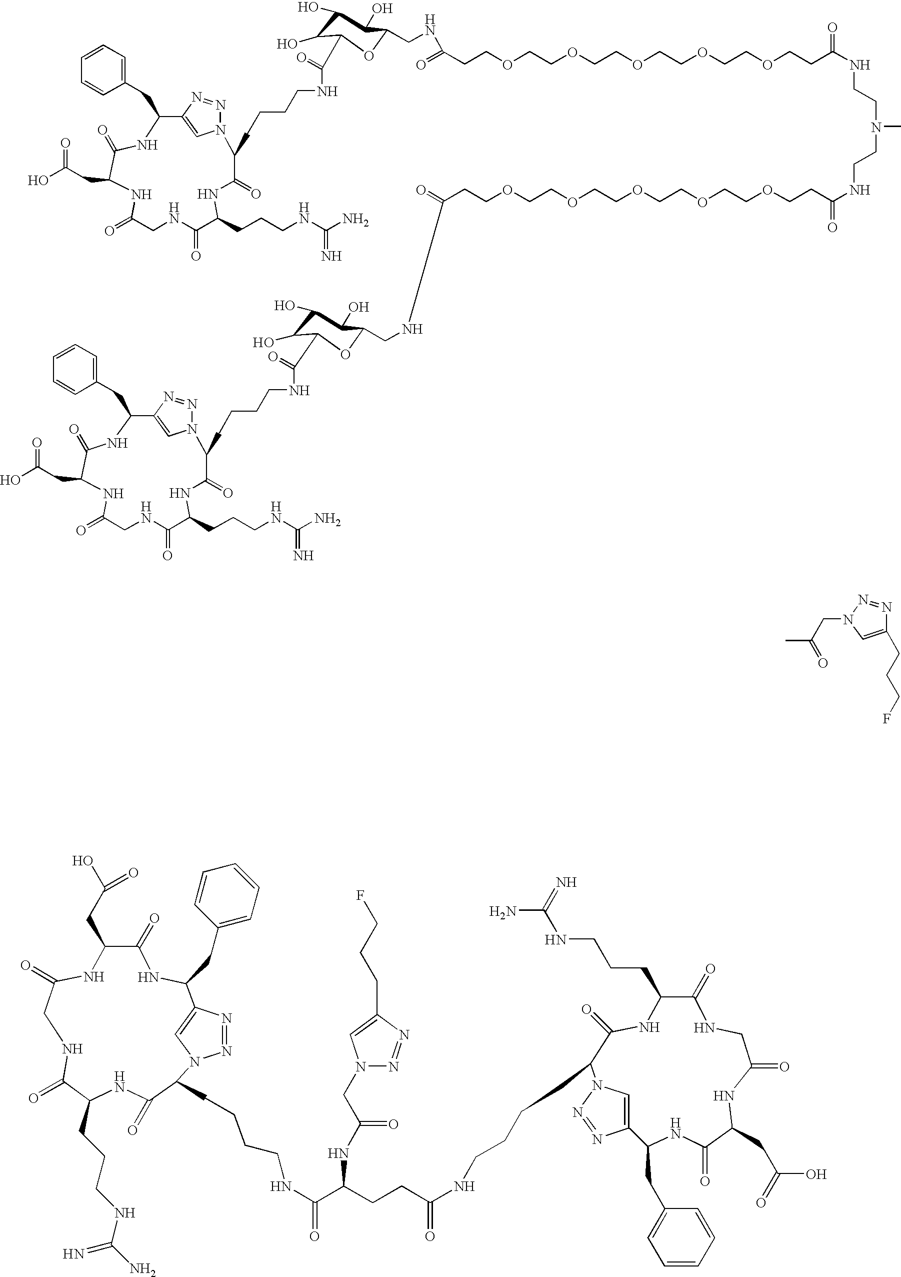

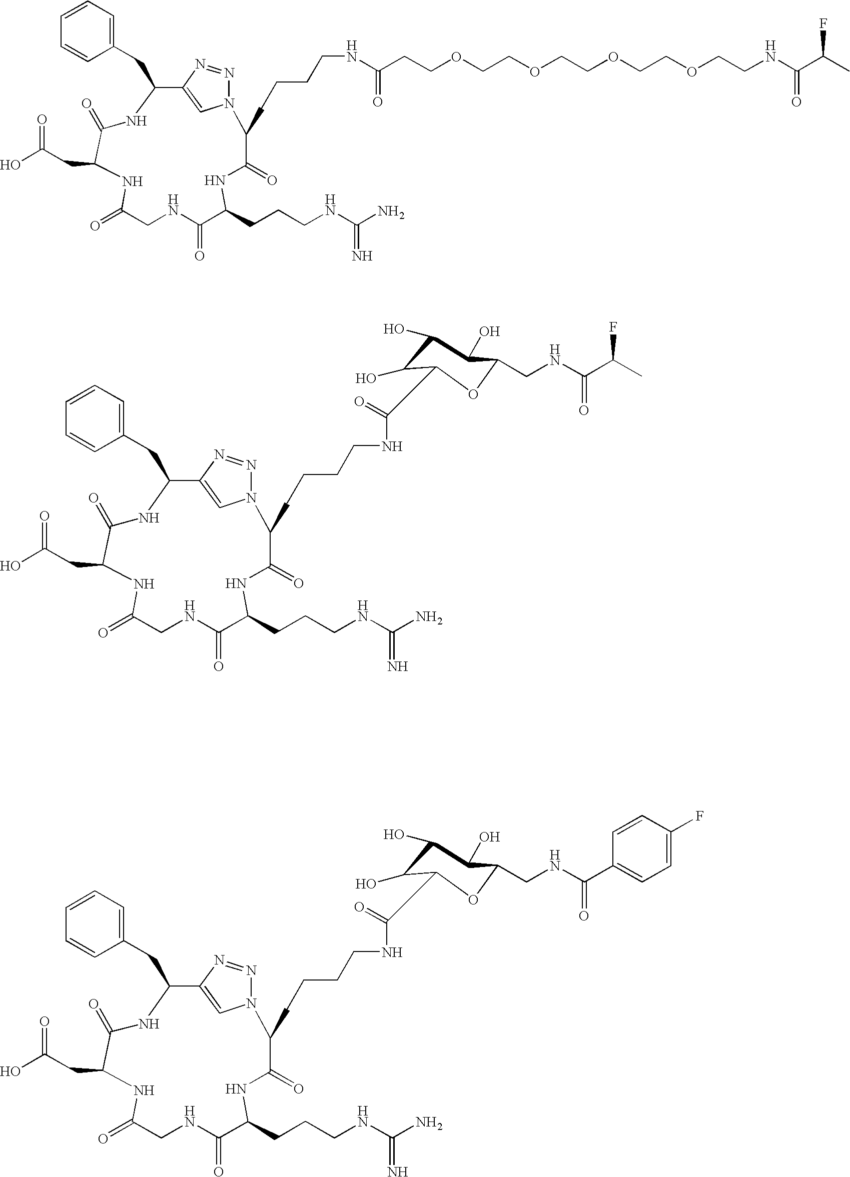

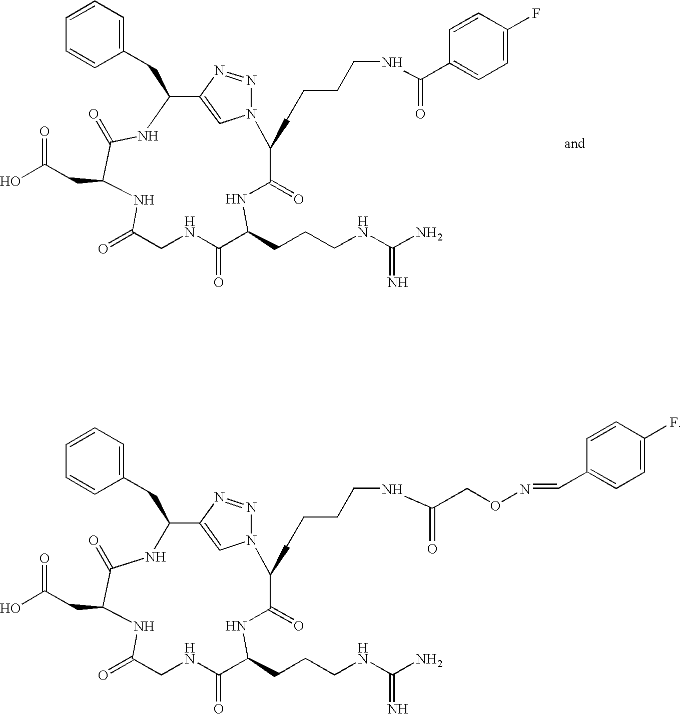

26. A pharmaceutical composition comprising a radiolabeled cyclic peptidomimetic of formula I: 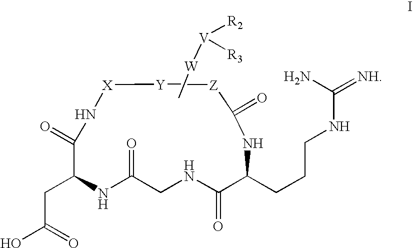
wherein
W is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;
V is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;
wherein at least one, but not both of W and V is a 5- or 6-membered heterocycle;
X is selected from the group consisting of —C₁-C₆ alkyl-(5-to 6-membered heterocycle)-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;
Y is selected from the group consisting of 5- or 6-membered heterocycle, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;
Z is selected from the group consisting of -(5- or 6-membered heterocycle)-C₁-C₆ alkyl-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;
any one of X, Y, or Z but not more than one of X, Y and Z is a 5- or 6-membered heterocycle;
where each R₁ is independently selected from the group consisting of a side chain of a natural amino acid and a side chain of an unnatural amino acid, wherein the natural amino acid and the unnatural amino acid is either in the D or L form;
R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene, carbocycle and heterocycle groups are each optionally substituted; and
optionally the fragment W—V(R₂)(R₃) is absent;
wherein at least one of W, X, Y, Z, R₂, and R₃ comprises a radionuclide selected from the group consisting of positron or gamma emitters;
and a pharmaceutically acceptable carrier.

27. A pharmaceutical composition comprising a radiolabeled cyclic peptidomimetic of formula II or formula IV: 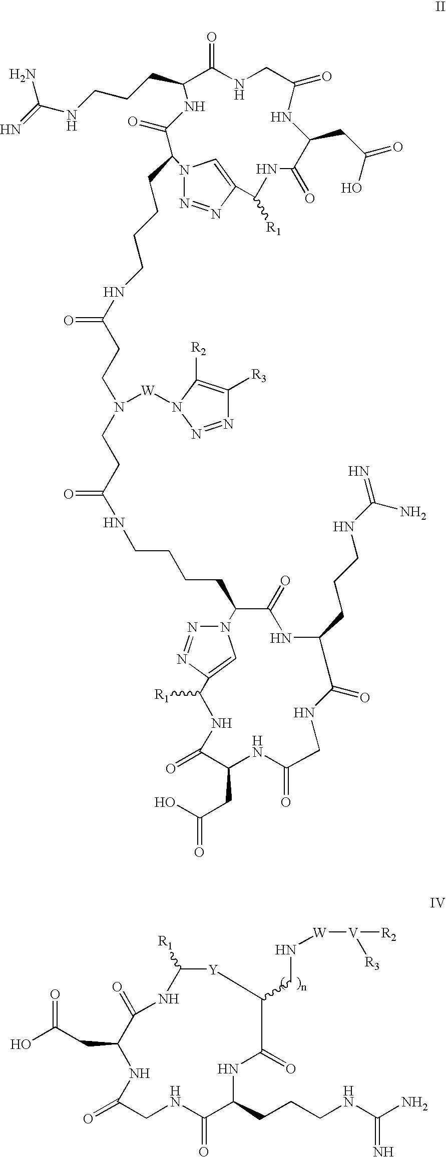
wherein
V is 1,2,3-triazolyl;
n is 1, 2, 3, 4 or 5;
each R₁ is independently selected from the group consisting of a side chain of a natural amino acid and a side chain of an unnatural amino acid, wherein the natural amino acid and the unnatural amino acid is either in the D or L form;
R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I and ¹³¹I; 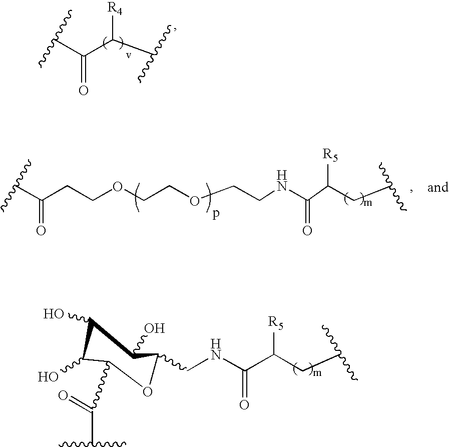
W is selected from the group consisting of
where p is 0 to 15;
v is 0, 1, 2, or 3;
m is 0, 1 or 2;
each R₄ and R₅ is independently selected from the group consisting of —H, and optionally substituted C₁-C₆ alkyl;
wherein the configuration of the chiral center that carries the R₅ substituent may be R or S or mixtures thereof;
and a pharmaceutically acceptable carrier.

28. A pharmaceutical composition comprising a radiolabeled cyclic peptidomimetic selected from the group consisting of: 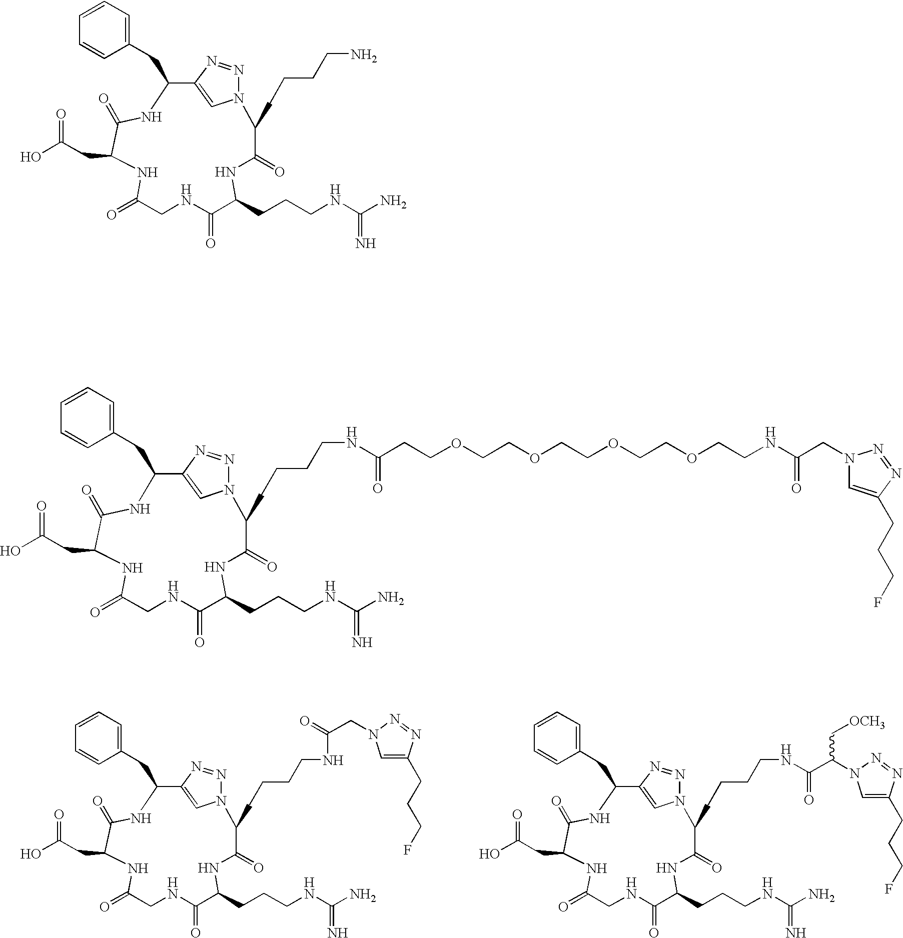 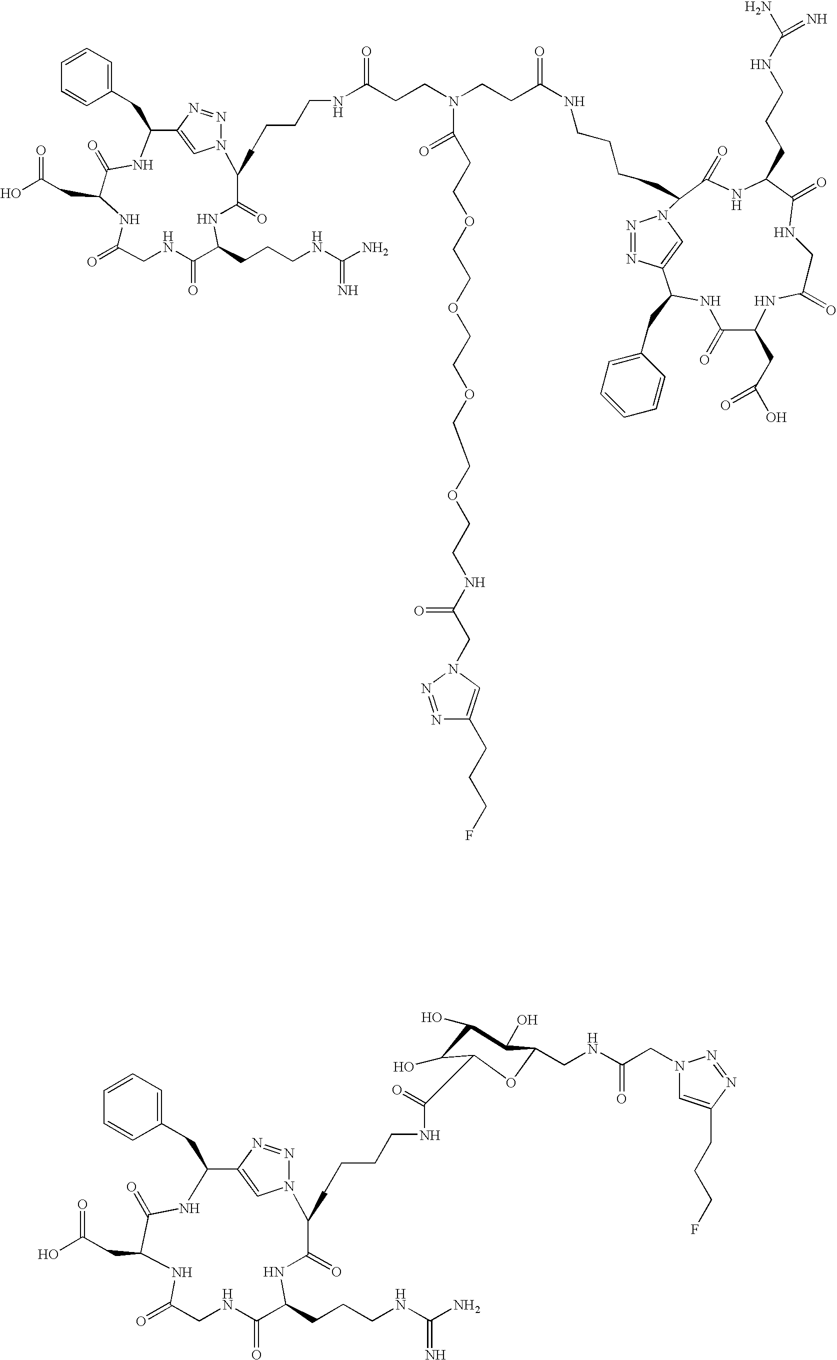 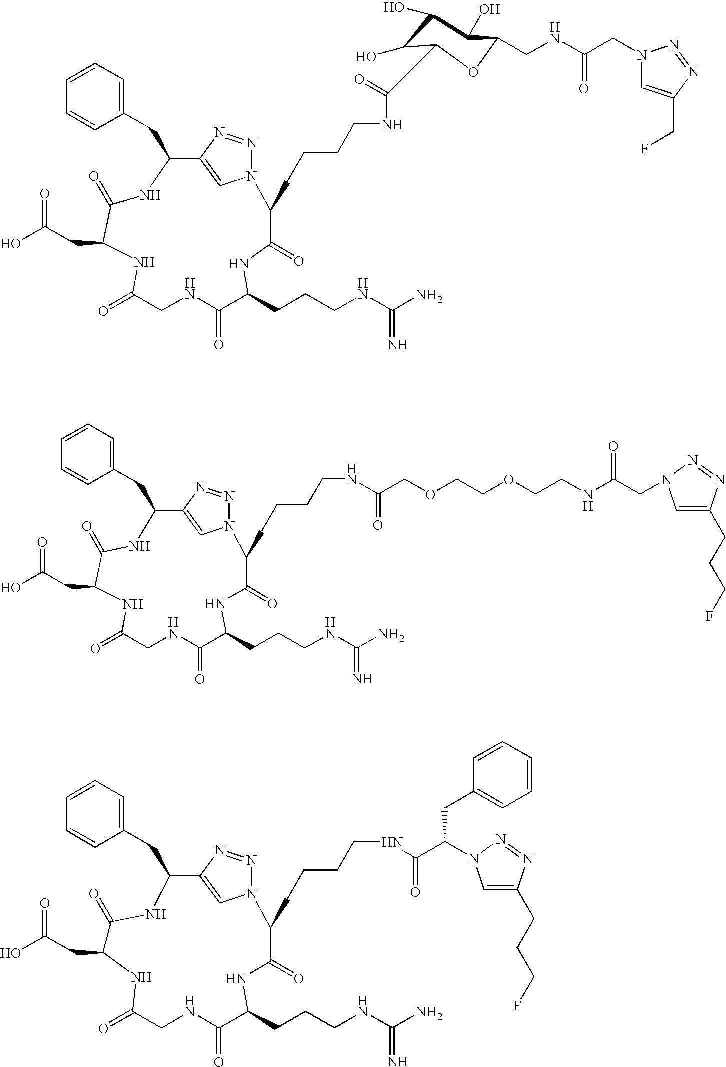 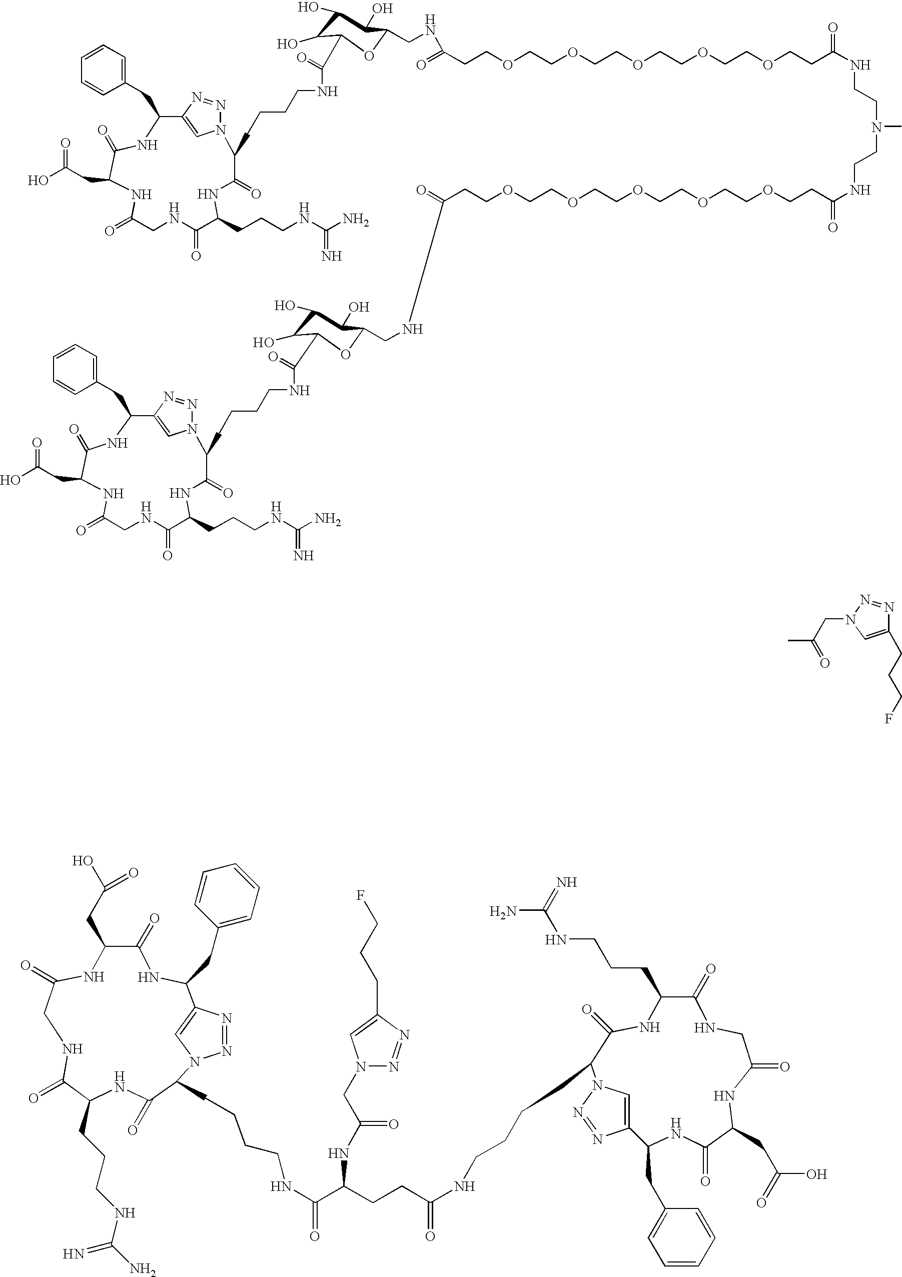 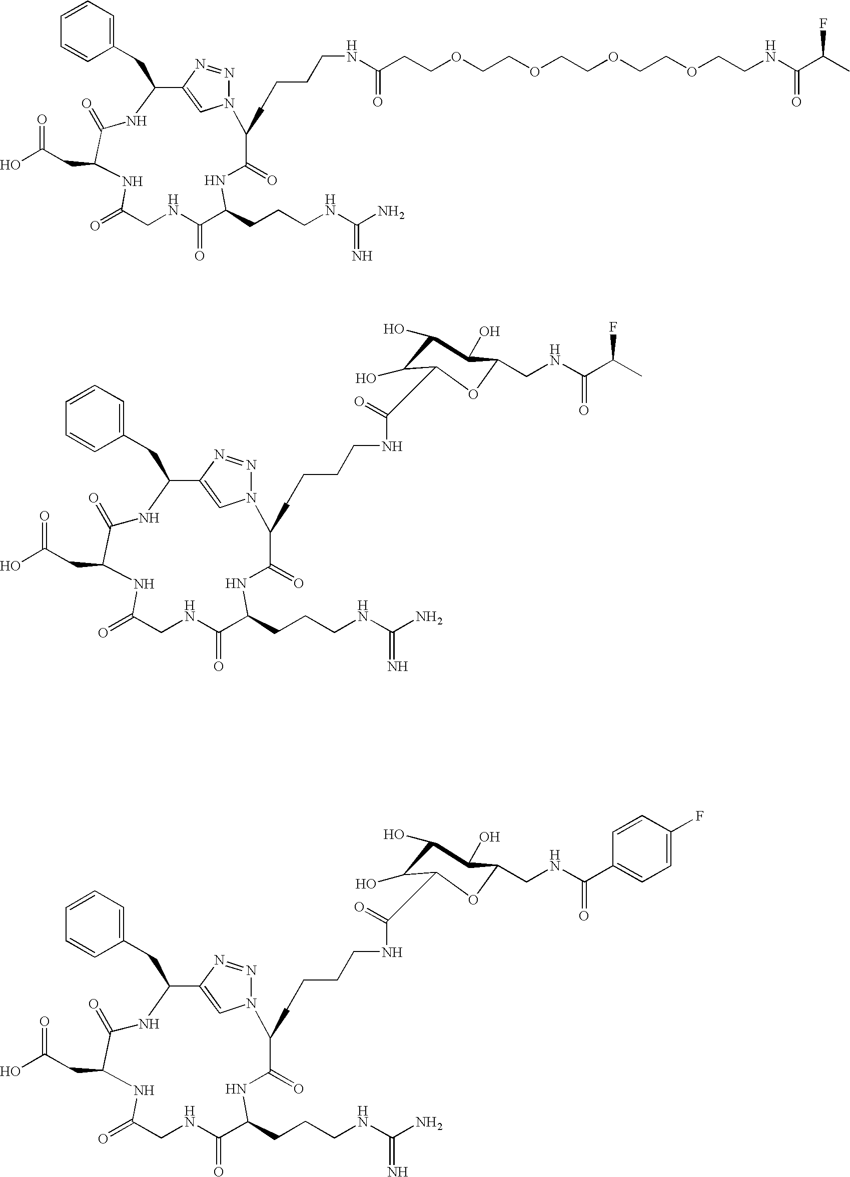 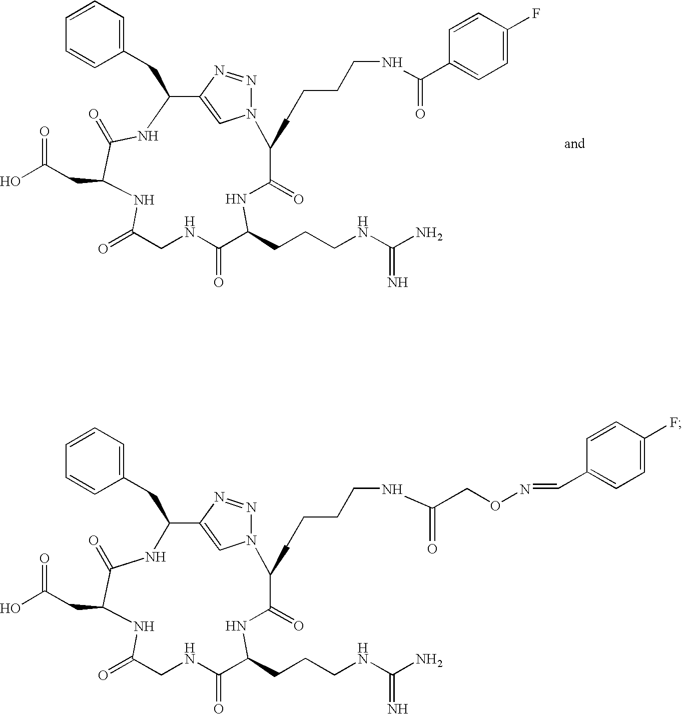
and a pharmaceutically acceptable carrier.

29. A method of monitoring the level of integrin αᵥβ₃ or visualizing integrin αᵥβ₃ expression within a body of a patient, the method comprising: (a) administering to the patient a radiolabeled cyclic peptidomimetic; and (b) employing a nuclear imaging technique selected from the group consisting of positron emission tomography (PET) and single photon emission computed tomography (SPECT) for monitoring or visualizing a distribution of the cyclic peptidomimetic within the body or within a portion thereof; wherein the radiolabeled cyclic peptidomimeticis of formula I: 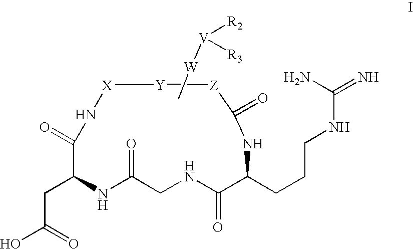
wherein
W is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;
V is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar-moiety;
wherein at least one, but not both of W and V is a 5- or 6-membered heterocycle;
X is selected from the group consisting of —C₂-C₆ alkyl-(5-to 6-membered heterocycle)-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;
Y is selected from the group consisting of 5- or 6-membered heterocycle, —C(H)(R₁)—, C₁-C₆ alkyl, C₂-C₆ alkenyl, C₂-C₆alkynyl, aryl, and aryl-(C₁-C₆ alkylene)- wherein the alkyl, alkenyl, alkynyl, aryl-alkylene groups are each optionally substituted;
Z is -(5- or 6-membered heterocycle)-C₁-C₆ alkyl-, —C(H)(R₁)—, C₁-C₆ alkyl, C₂-C₆ alkenyl, C₂-C₆ alkynyl, aryl, aryl-(C₁-C₆ alkylene)- wherein the alkyl, alkenyl, alkynyl, aryl-alkylene groups are each optionally substituted;
any one of X, Y, or Z but not more than one of X, Y and Z is a 5- or 6-membered heterocycle;
where each R₁ is independently selected from the group consisting of a side chain of a natural amino acid and a side chain of an unnatural amino acid, wherein the natural amino acid and the unnatural amino acid is either in the D or L form;
R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene, carbocycle and heterocycle groups are each optionally substituted; and
optionally the fragment W—V(R₂)(R₃) is absent;
wherein at least one of W, X, Y, Z, R₂, and R₃ comprises a radionuclide selected from the group consisting of positron or gamma emitters.

30. A method of monitoring the level of integrin αᵥβ₃ or visualizing integrin αᵥβ₃ expression within a body of a patient, the method comprising: (a) administering to the patient a radiolabeled cyclic peptidomimetic; and (b) employing a nuclear imaging technique selected from the group consisting of positron emission tomography (PET) and single photon emission computed tomography (SPECT) for monitoring or visualizing a distribution of the radiolabeled cyclic peptidomimetic within the body or within a portion thereof; wherein the radiolabeled cyclic peptidomimetic is of formula II or formula IV: 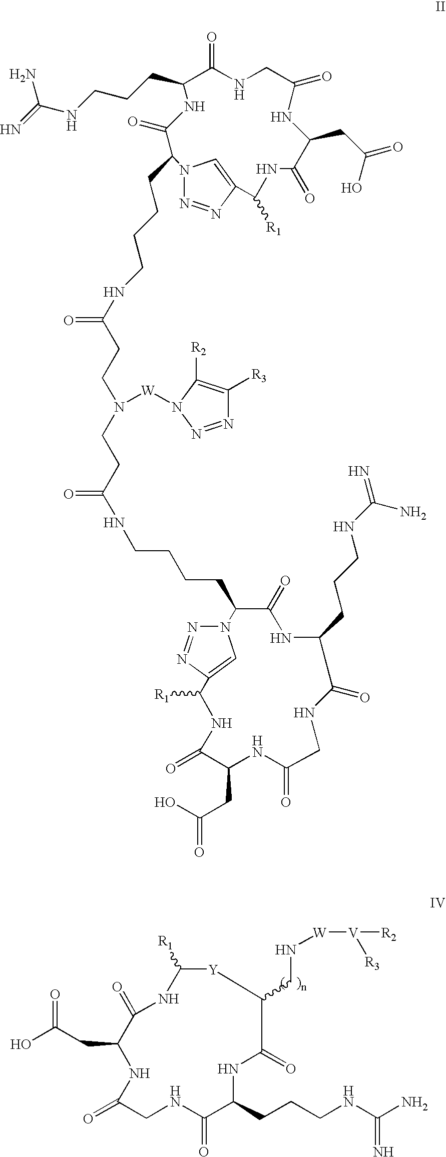
wherein
V is 1,2,3-triazolyl;
n is 1, 2, 3, 4 or 5;
each R₁ is independently selected from the group consisting of a side chain of a natural amino acid and a side chain of an unnatural amino acid, wherein the natural amino acid and the unnatural amino acid is either in the D or L form;
R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I and ¹³¹I;
W is selected from the group consisting of 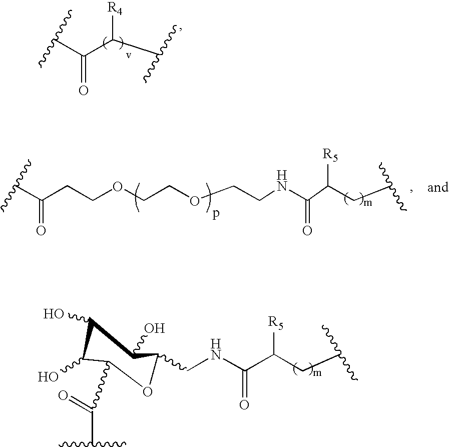
where p is 0 to 15;
v is 0, 1, 2, or 3;
m is 0, 1 or 2;
each R₄ and R₅ is independently selected from the group consisting of —H, and optionally substituted C₁-C₆ alkyl;
wherein the configuration of the chiral center that carries the R₅ substituent may be R or S or mixtures thereof.

31. A method of monitoring the level of integrin αᵥβ₃ or visualizing integrin αᵥβ₃ expression within a body of a patient, the method comprising: (a) administering to the patient a radiolabeled cyclic peptidomimetic; and (b) employing a nuclear imaging technique selected from the group consisting of positron emission tomography (PET) and single photon emission computed tomography (SPECT) for monitoring or visualizing a distribution of the radiolabeled cyclic peptidomimetic within the body or within a portion thereof; wherein the radiolabeled peptidomimetic selected from the group consisting of:

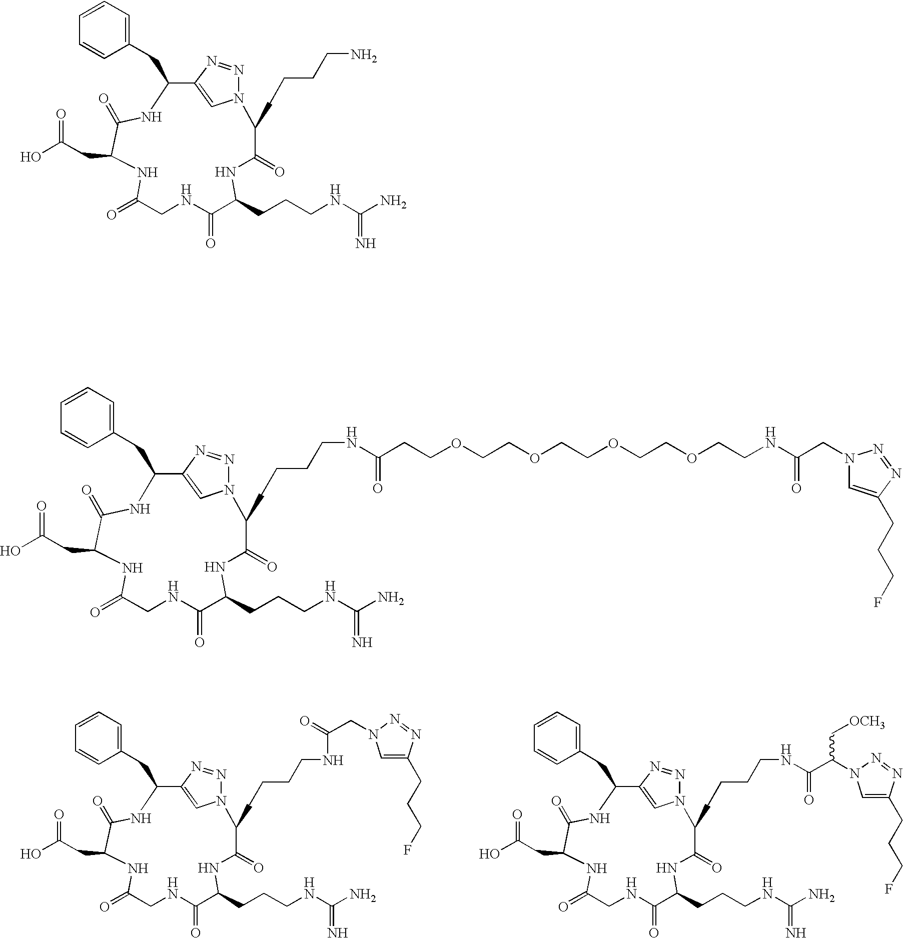

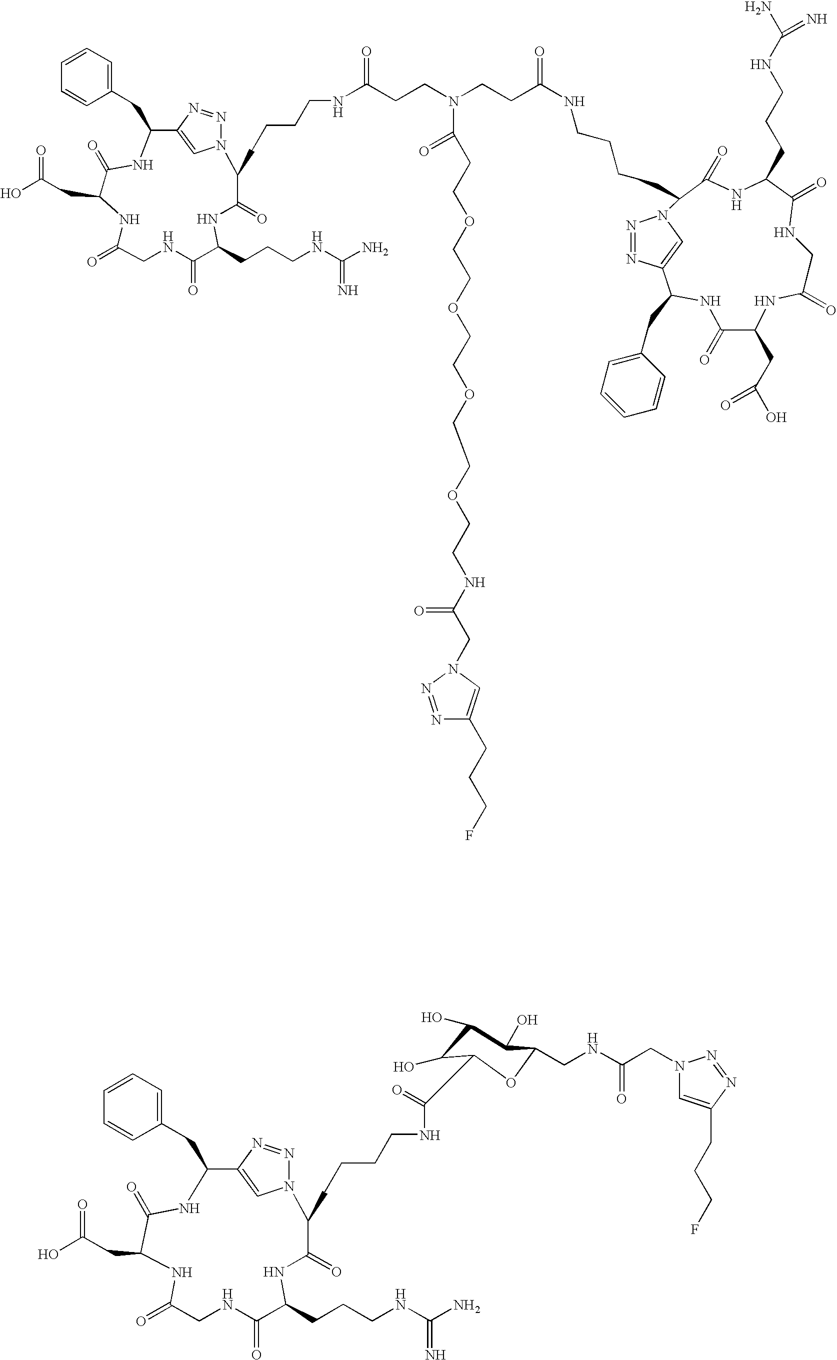

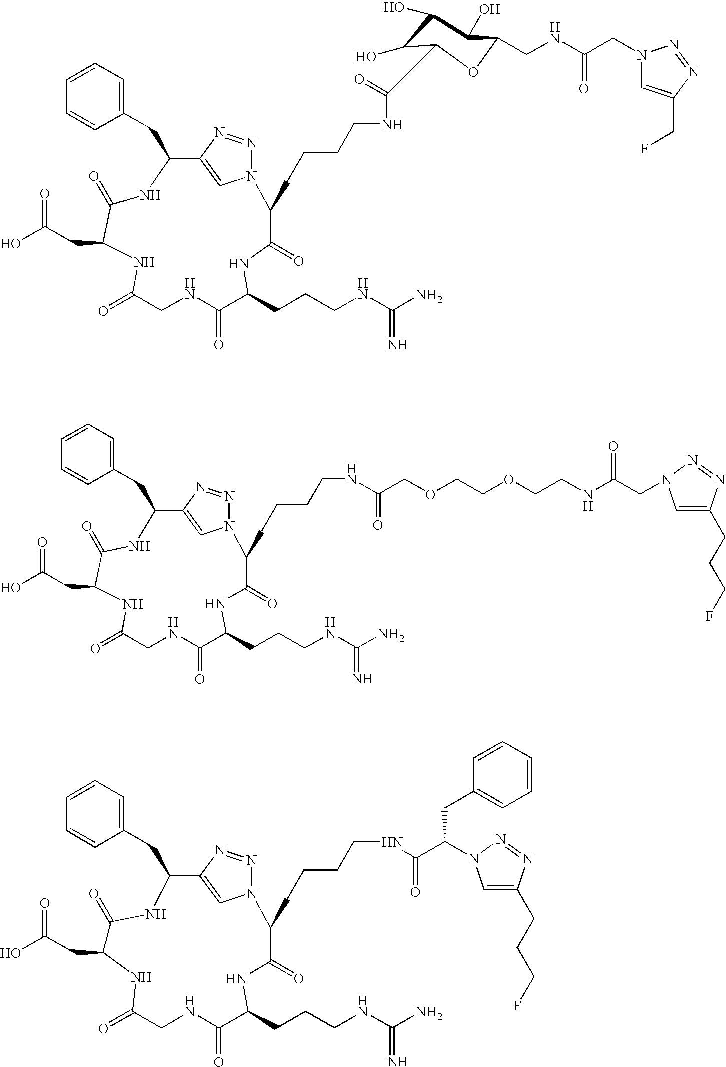

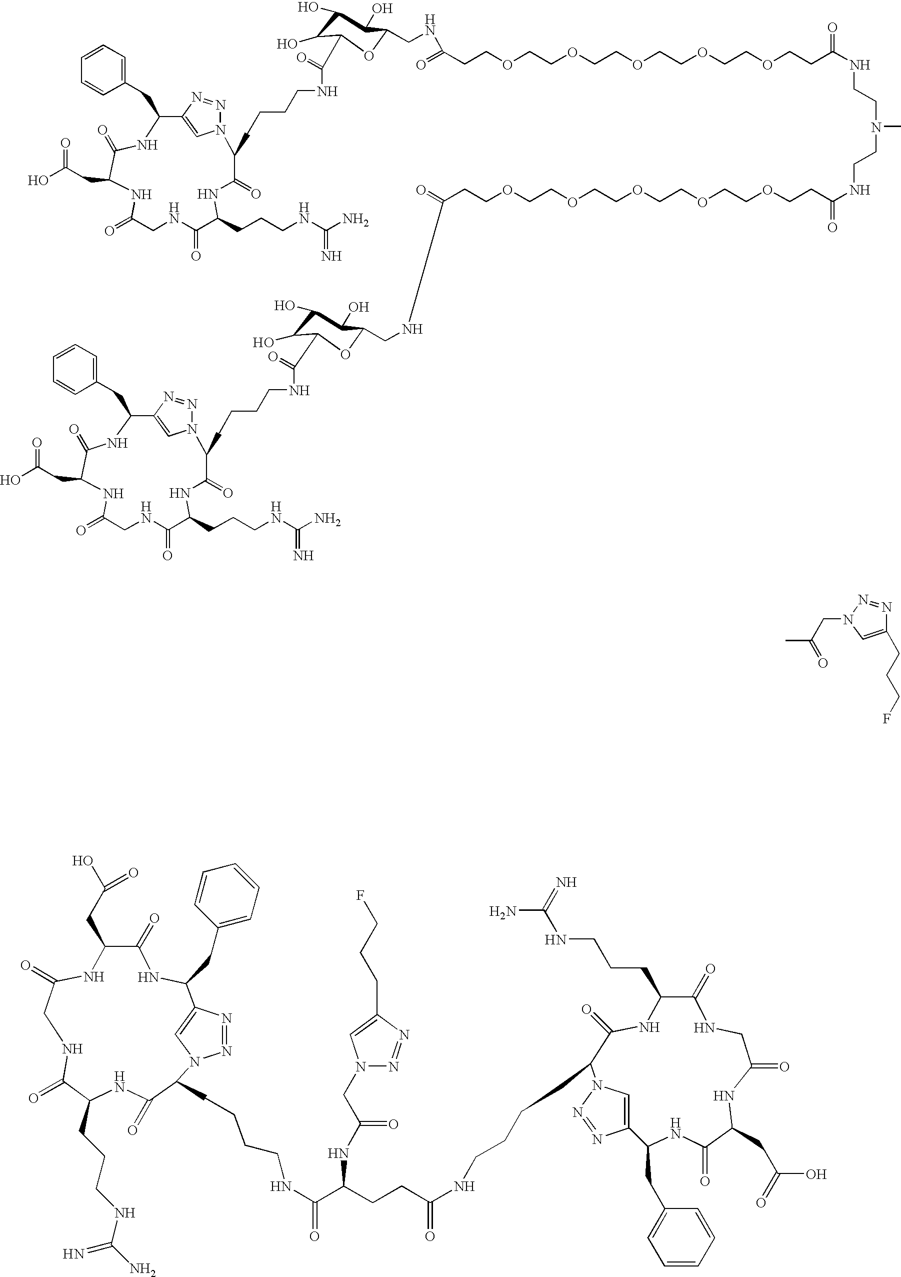

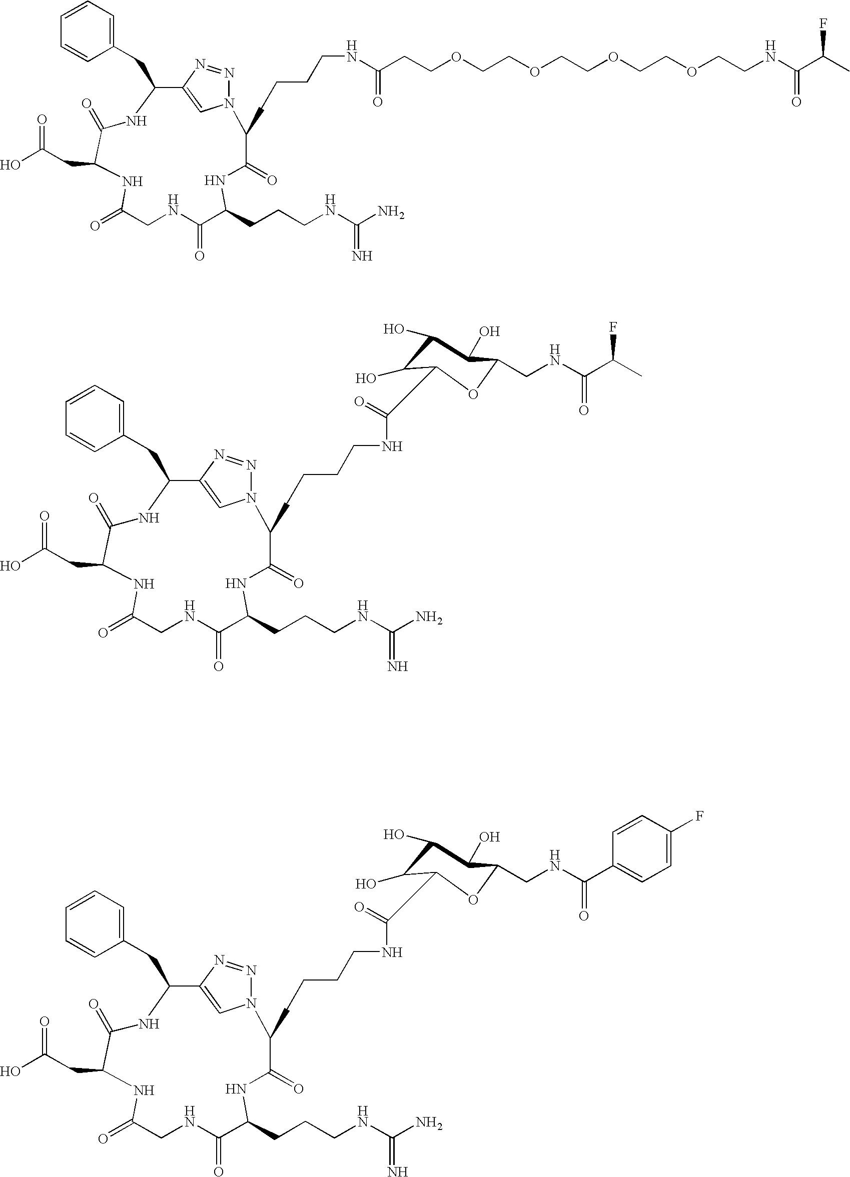

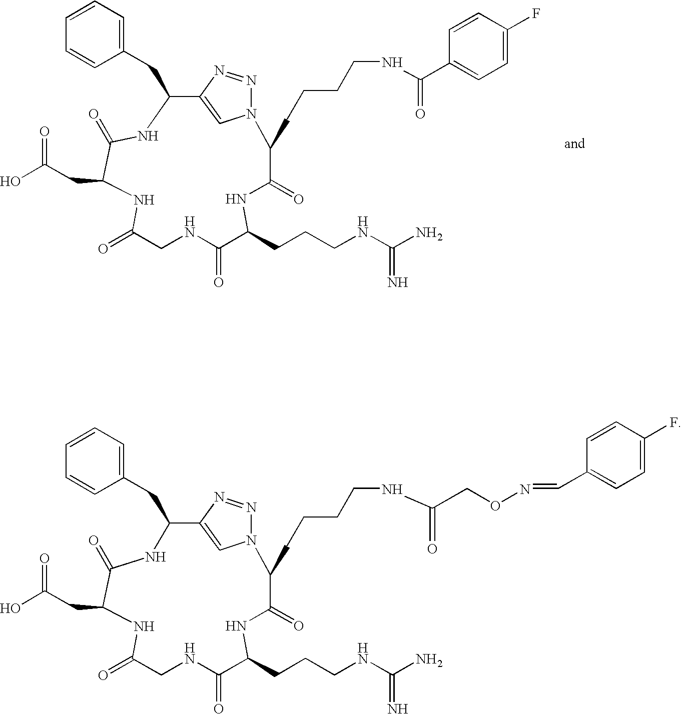

32. A method for imaging of blood vessel growth in solid tumors based on expression of integrin αᵥβ₃ within the body of a patient, the method comprising: (a) administering to the patient radiolabeled cyclic peptidomimetic; (b) employing a nuclear imaging technique selected from the group consisting of positron emission tomography (PET) and single photon emission computed tomography (SPECT) for imaging a distribution of the radiolabeled cyclic peptidomimetic within the body or within a portion thereof; and c) correlating the distribution of the radiolabeled cyclic peptidomimetic to the growth of blood vessels in solid tumors, wherein the radiolabeled cyclic peptidomimetic is of formula I: 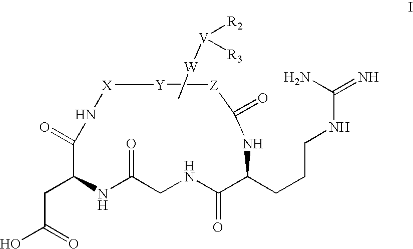
wherein
W is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;
V is a 5- or 6-membered heterocycle or a linker comprising a hydrophilic moiety selected from the group consisting of hydroxyl, carbonyl, sulfonamide, sulfonate, phosphate, polar amino acid moiety, PEG moiety, sugar mimetic, and sugar moiety;
wherein at least one, but hot both of W and V is a 5- or 6-membered heterocycle;
X is selected from the group consisting of —C₁-C₆ alkyl-(5-to 6-membered heterocycle)-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;
Y is selected from the group consisting of 5- or 6-membered heterocycle, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;
Z is selected from the group consisting of -(5- or 6-membered heterocycle)-C₁-C₆ alkyl-, —C(H)(R₁)—, C₁-C₆ alkyl, C₂-C₆ alkenyl, C₂-C₆ alkynyl, aryl, and aryl-(C₁-C₆ alkylene)- wherein the alkyl, alkenyl, alkynyl, aryl-alkylene groups are each optionally substituted;
any one of X, Y, or Z but not more than one of X, Y and Z is a 5- or 6-membered heterocycle;
where each R₁ is independently selected from the group consisting of a side chain of a natural amino acid and a side chain of an unnatural amino acid, wherein the natural amino acid and the unnatural amino acid is either in the D or L form;
R₂ and R₃ are each independently selected from the group consisting of H, C₁-C₆ alkyl, C₂-C₆ alkenyl, C₂-C₆ alkynyl, aryl, aryl-(C₁-C₆ alkylene)-, a 3- to 7-membered carbocycle, and a 3- to 7-membered heterocycle, wherein the alkyl, alkenyl, alkynyl, aryl-alkylene, carbocycle and heterocycle groups are each optionally substituted; and
optionally the fragment W—V(R₂)(R₃) is absent;
wherein at least one of W, X, Y, Z, R₂, and R₃ comprises a radionuclide selected from the group consisting of positron or gamma emitters.

33. A method for imaging of blood vessel growth in solid tumors based on expression of integrin αᵥβ₃ within the body of a patient, the method comprising: (a) administering to the patient radiolabeled cyclic peptidomimetic; (b) employing a nuclear imaging technique selected from the group consisting of positron emission tomography (PET) and single photon emission computed tomography (SPECT) for imaging a distribution of the radiolabeled cyclic peptidomimetic within the body or within a portion thereof; and c) correlating the distribution of the radiolabeled cyclic peptidomimetic to the growth of blood vessels in solid tumors, wherein the radiolabeled cyclic peptidomimetic is of formula II or formula IV: 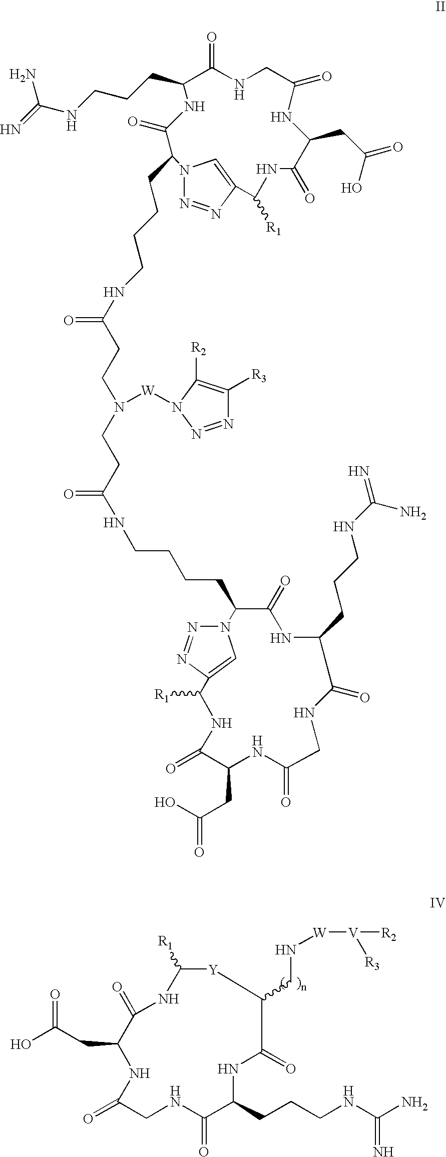
wherein
V is 1,2,3-triazolyl;
n is 1, 2, 3, 4 or 5;
each R₁ is independently selected from the group consisting of a side chain of a natural amino acid and a side chain of an unnatural amino acid, wherein the natural amino acid and the unnatural amino acid is either in the D or L form;
R₂ and R₃ are each independently selected from the group consisting of —H, C₁-C₆ alkyl, C₂-C₆ alkenyl, and C₂-C₆ alkynyl, wherein the alkyl, alkenyl, and alkynyl groups are each optionally substituted, wherein R₂ and R₃ are not both H; and either R₂ or R₃, or both R₂ and R₃ comprise a radionuclide selected from the group consisting of ¹¹C, ¹³N, ¹⁵O, ¹⁸F, ⁷⁵Br, ¹²⁴I, ¹²⁵I and ¹³¹I;
W is selected from the group consisting of 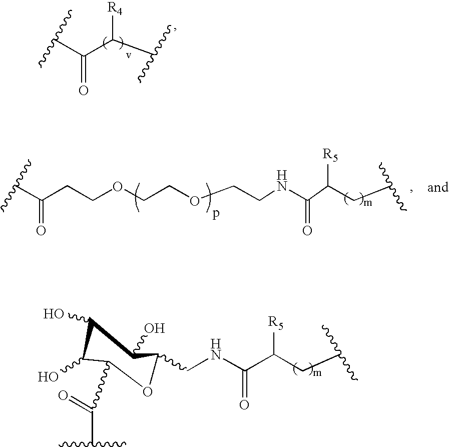
where p is 0 to 15;
v is 0, 1, 2, or 3;
m is 0, 1 or 2;
each R₄ and R₅ is independently selected from the group consisting of —H, and optionally substituted C₁-C₆ alkyl;
wherein the configuration of the chiral center that carries the R₅ substituent may be R or S or mixtures thereof.

34. A method for imaging of blood vessel growth in solid tumors based on expression of integrin αᵥβ₃ within the body of a patient, the method comprising: (a) administering to the patient radiolabeled cyclic peptidomimetic; (b) employing a nuclear imaging technique selected from the group consisting of positron emission tomography (PET) and single photon emission computed tomography (SPECT) for imaging a distribution of the radiolabeled cyclic peptidomimetic within the body or within a portion thereof; and c) correlating the distribution of the radiolabeled cyclic peptidomimetic to the growth of blood vessels in solid tumors, wherein the radiolabeled cyclic peptidomimetic is selected from the group consisting of: 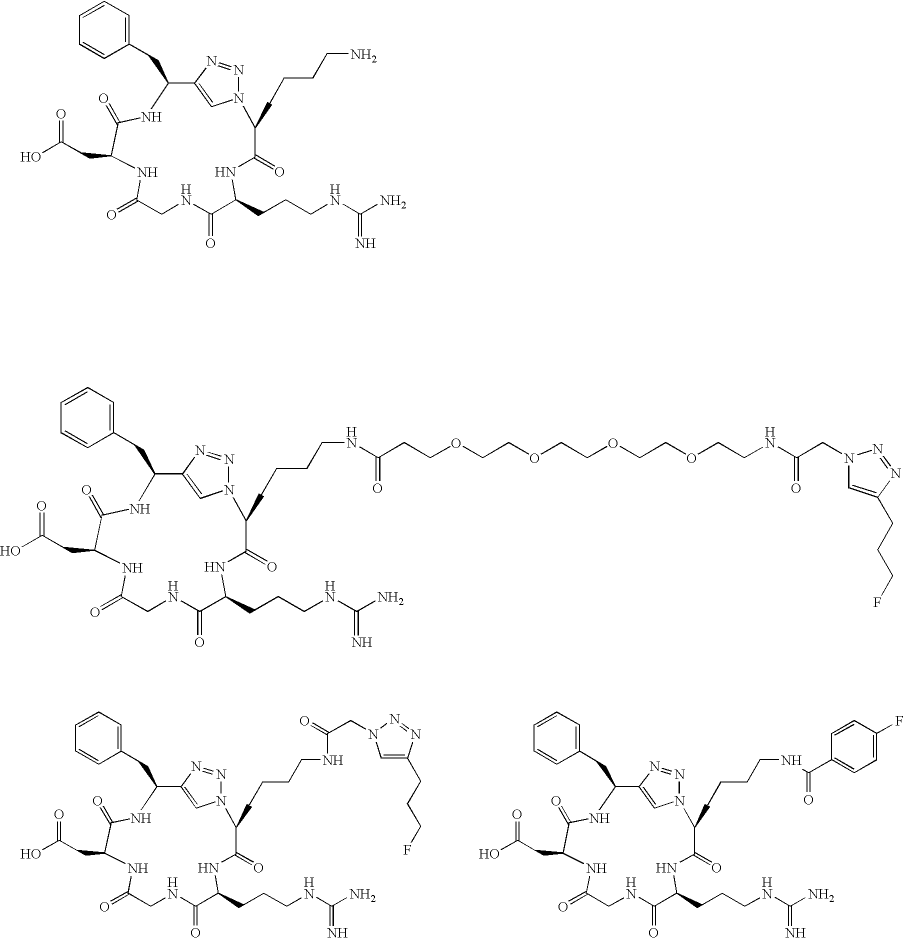 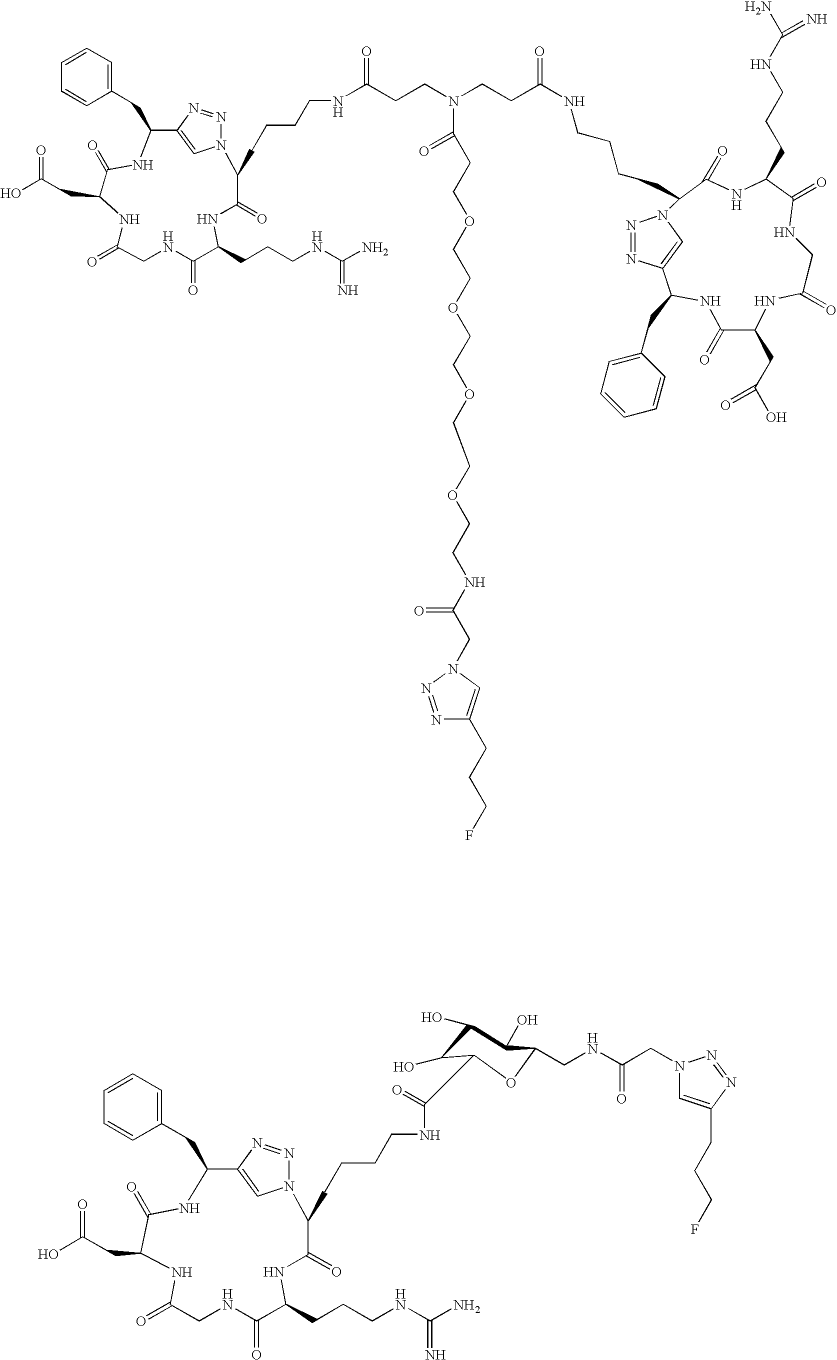 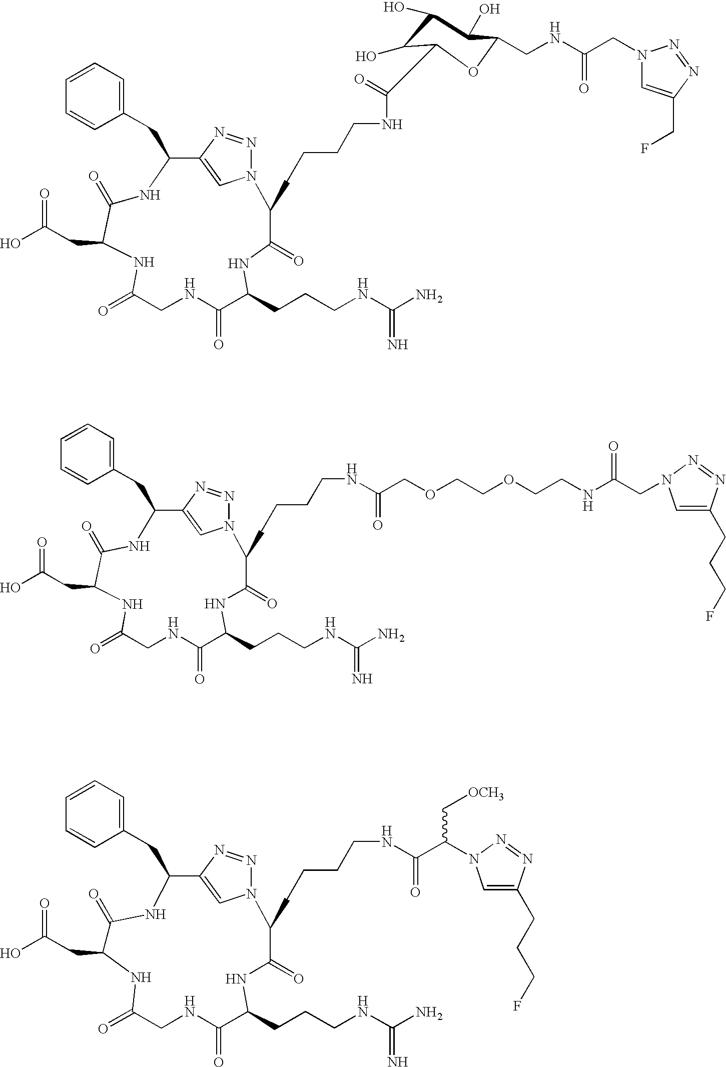 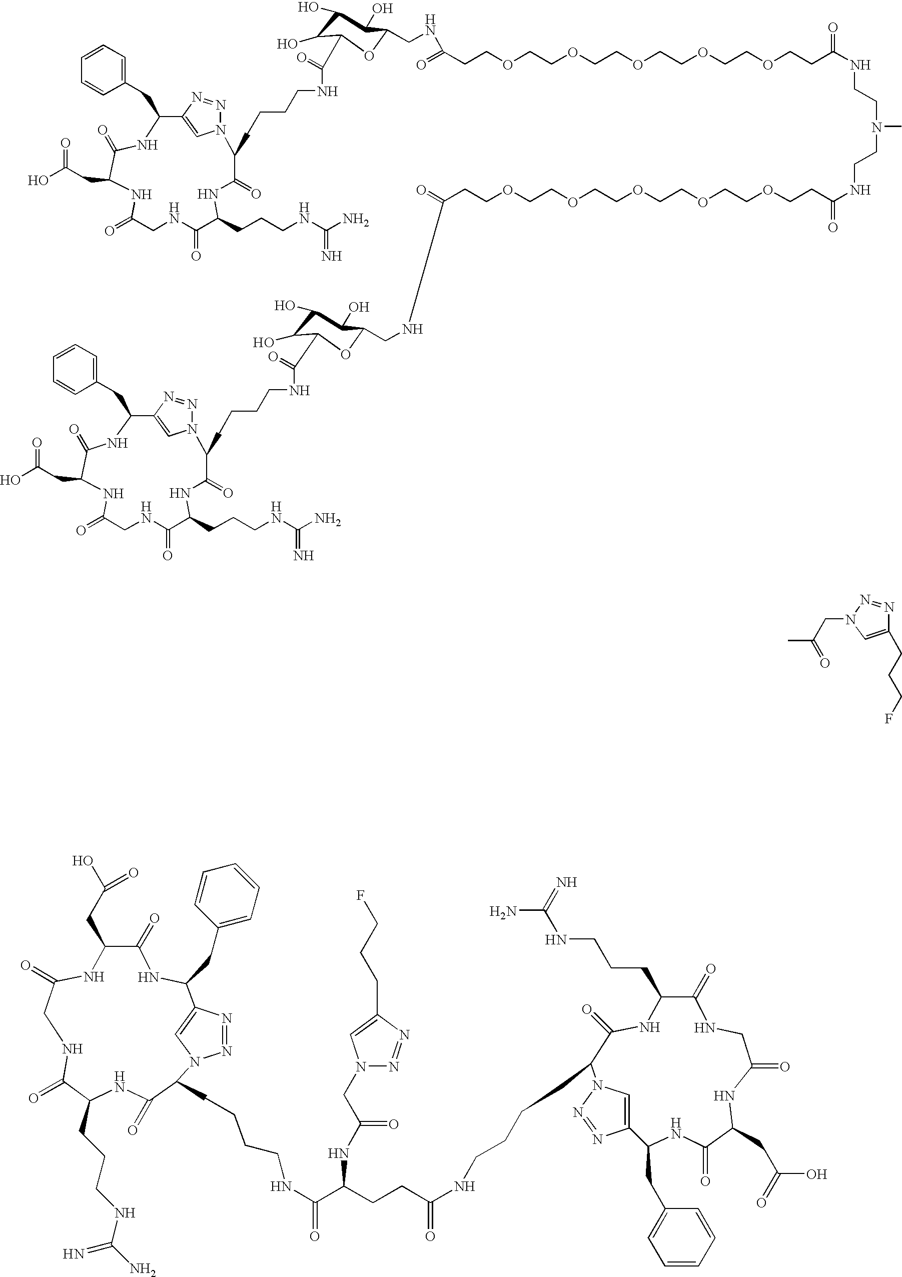 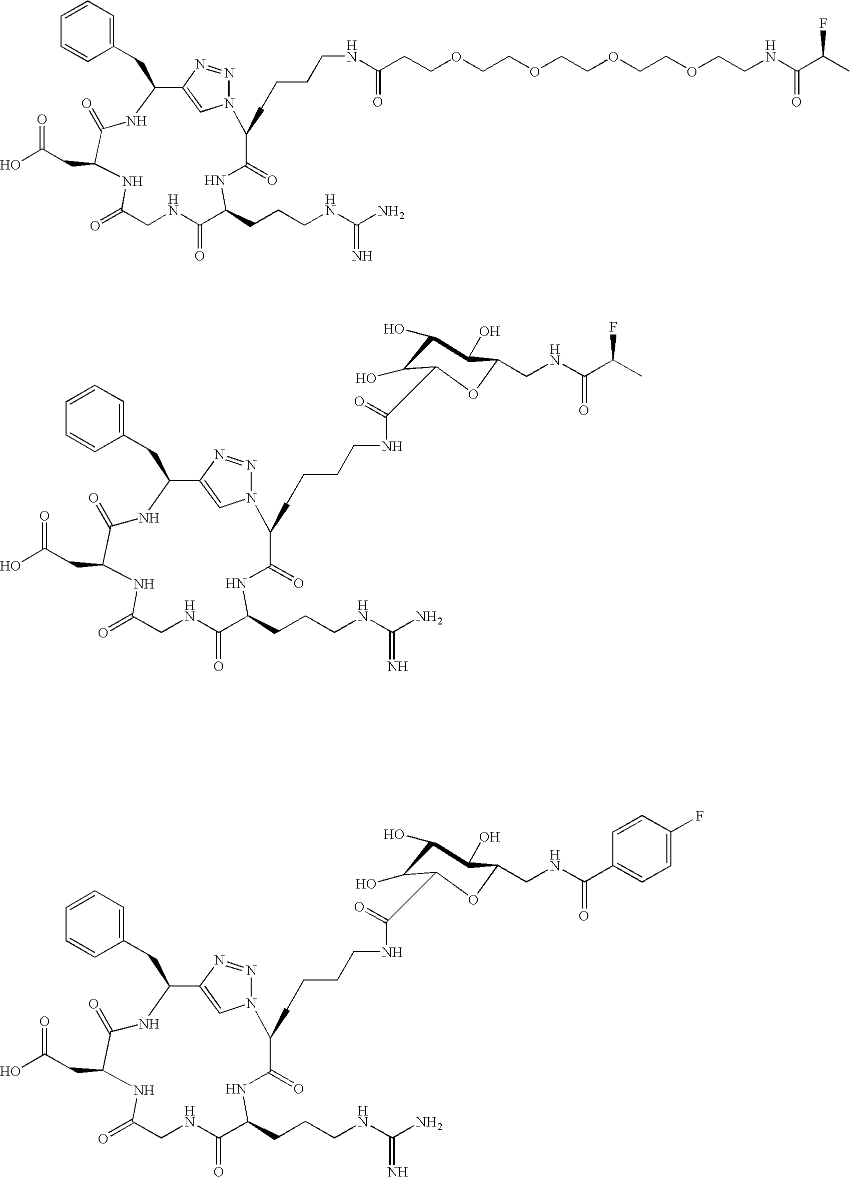 [chemical structure]
